(19) United States
(12) Patent Application Publication
(10) Pub. No.: US 2004/0229930 A1
(43) Pub. Date: Nov. 18, 2004

(54) Formulations comprising a basic indolinone compound

(75) Inventor: Paolo Gatti, Genova (IT)

Correspondence Address: FOLEY AND LARDNER, SUITE 500, 3000 K STREET NW, WASHINGTON, DC, 20007, US

(73) Assignee: Pharmacia Italia S.p.A.

(21) Appl. No.: 10/658,801

(22) Filed: Sep. 10, 2003

Related U.S. Application Data
(60) Provisional application No. 60/421,133, filed on Sep. 10, 2002.

(51) Int. Cl. A61K 31/404; A61K 9/20

(52) U.S. Cl. 514/414; 424/465

(57) ABSTRACT

The present invention features formulations of indolinones. The formulation is suitable for parenteral or oral administration, wherein the formulation comprises an indolinone, and a pharmaceutically acceptable carrier therefor. The formulations and the compounds themselves are useful for the treatment of protein kinase related disorders.

### CROSS-REFERENCE TO RELATED APPLICATIONS

This application claims priority under 35 U.S.C. 119(e) to U.S. Provisional Application Serial No. 60/421,133, filed Sep. 10, 2002, the disclosure of which is incorporated by reference herein in its entirety.

### FIELD OF THE INVENTION

The instant invention provides formulations for indolinones such as pyrrole substituted 2-indolinones. The invention further contemplates pharmaceutically active salts, prodrugs, derivatives, and analogs of the indolinones. Also provided are methods of making and using the formulations of the invention.

### BACKGROUND OF THE INVENTION

The following description of the background of the invention is provided to aid in understanding the invention, but is not admitted to describe or constitute prior art to the invention.

Various methods are available for administering therapeutic agents to a patient. Such methods include parenteral, oral, ocular, nasal, topical, and transmucosal administration.

There is a need in the art for a stable, uniform formulation of indolinones which can be readily formed into dosage forms and which is substantially free of the disadvantages of formulations disclosed in the prior art. An object of the invention is to provide a stable indolinone formulation which can be readily formed into an oral capsule or tablet.

### SUMMARY OF THE INVENTION

In one aspect, the invention relates to a solid formulation, where the formulation comprises 5-60% w/w of an indolinone of formula I:

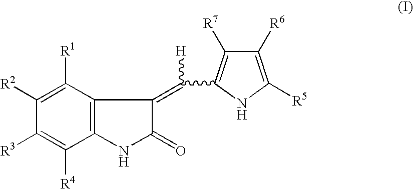

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or pharmaceutically active salts of the compound of formula I; and

a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants.

In a second aspect, the invention relates to a solid formulation, where the formulation comprises 5-60% w/w of an indolinone of formula I:

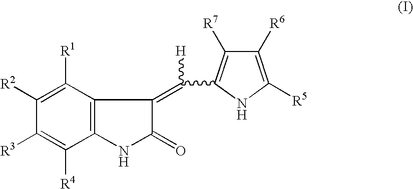

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I; and

a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants;

with the proviso that said formulation does not comprise a surfactant and/or a flow enhancer.

In a third aspect, the invention relates to a solid formulation, where the formulation consists essentially of 5-60% w/w of an indolinone of formula I:

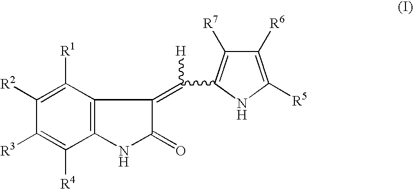

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)R¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—; with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I; and

a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmnaceutically acceptable lubricants.

In a fourth aspect, the invention relates to a solid formulation, where the formulation comprises 5-60% w/w of the malate salt of an indolinone of formula I:

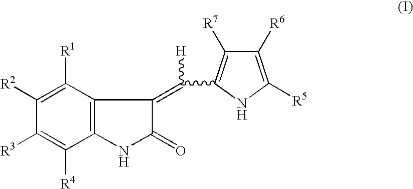

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)R¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—; with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹ )(CH₂), R¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; and

a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants.

In a fifth aspect, the invention relates to a solid formulation, where the formulation comprises an indolinone compound of formula I:

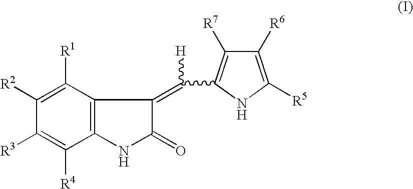

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, (CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I;

wherein the bulk density of said formulation is at least about 0.50 kg/L.

In a sixth aspect, the invention relates to a solid formulation, where the formulation comprises an indolinone compound of formula I:

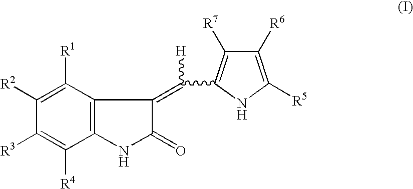

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, (CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I;

wherein said formulation is in particulate form, and wherein no more than 55% of the particles have a size less than 250 microns.

In a seventh aspect, the invention relates to a solid formulation, where the formulation comprises an indolinone compound of formula I:

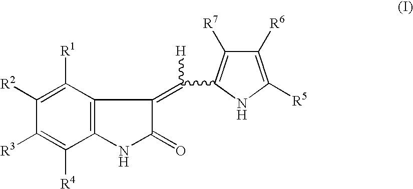

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴ NR¹³C(O)R¹⁴ , —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I;

wherein said formulation is in particulate form, and wherein the mean particle size is between 106 and 250 microns.

In an eight aspect, the invention relates to a solid formulation, where the formulation comprises the malate salt of an indolinone compound of formula I:

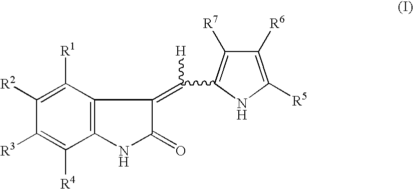

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4;

wherein the bulk density of said solid formulation is about 2 to about 8 fold higher than the bulk density of the malate salt of the indolinone compound by itself.

In a ninth aspect, the invention relates to a solid formulation, where the formulation comprises 15-40% w/w of an indolinone of formula I:

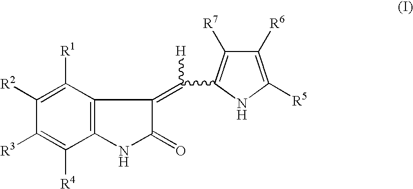

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—; with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R^{15,} —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I; and

a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants.

In a preferred embodiment, the formulation of the ninth aspect of the invention comprises mannitol as the diluent, polyvinylpyrrolidone as the binder, crosscaramellose sodium as the disintegrant and magnesium stearate as the lubricant.

In a preferred embodiment, the compound of formula I in the ninth aspect of the invention is:

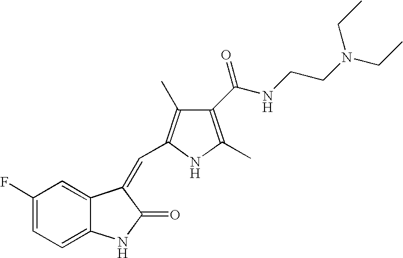

or a malate salt thereof.

In a preferred embodiment, the formulation of the ninth aspect of the invention comprises 40% w/w of an indolinone of formula I, or a pharmaceutically acceptable salt thereof, 47.5% w/w mannitol, 6% w/w croscaramellose sodium, 5% w/w povidone and 1.5% w/w magnesium stearate.

In a preferred embodiment, the formulation of the ninth aspect of the invention does not comprise a surfactant and/or a flow enhancer.

In a tenth aspect, the invention relates to a solid formulation which comprises an indolinone compound of formula I:

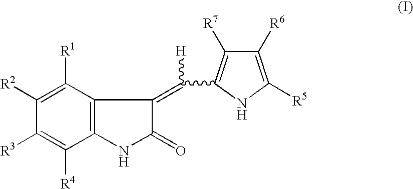

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O³¹ )R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I;

wherein the bulk density of said formulation is at least about 0.50 kg/L, 0.55, 0.56, 0.57, 0.58, 0.59. 0.60, 0.61, 0.62, 0.63, 0.64, 0.65, 0.66, 0.67, 0.69 or 0.7 kg/L.

In a preferred embodiment, the formulation of the tenth aspect of the invention has a ratio of tap density to bulk density of from about 1.10 to about 1.30, about 1.10 to about 1.25, or about 1.10 to about 1.20, or about 1.10 to about 1.15.

In a preferred embodiment, the compound of formula I in the tenth aspect of the invention is:

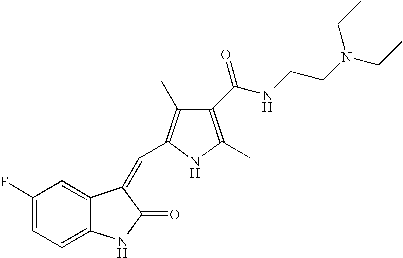

or a malate salt thereof.

In a preferred embodiment, the formulation of the tenth aspect of the invention comprises 15-40% of the indolinone compound.

In a preferred embodiment, the for nulation of the tenth aspect of the invention the formulation comprises 40% w/w of an indolinone of formula I, or a pharmaceutically acceptable salt thereof, 47.5% w/w mannitol, 6% w/w croscarainellose sodium, 5% w/w povidone and 1.5% w/w magnesium stearate.

In an eleventh aspect the invention relates to a solid formulation comprising an indolinone compound of formula I:

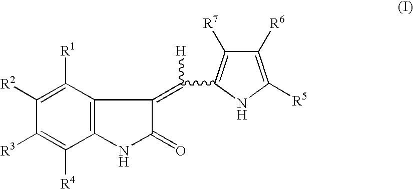

wherein:

R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is —N(R¹¹)(CH₂)ₙR¹² or —NHCH₂CH(OH)CH₂R₁₂;

R¹¹ is selected from the group consisting of hydrogen and alkyl;

R¹² is selected from the group consisting of —NR¹³R¹⁴, —N⁺(O⁻)R¹³R¹⁴, —N(OH)R¹³, and —NHC(O)R¹³;

R¹³ and R¹⁴ are independently selected from the group consisting of hydrogen, alkyl, cyanoalkyl, cycloalkyl, aryl and heteroaryl; or

R¹³ and R¹⁴ may combine to form a heteroalicyclic or heteroaryl group;,

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴,

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4; or

pharmaceutically active salts of the compound of formula I;

wherein said formulation is in particulate form, and wherein no more than 55% of the particles have a size less than 250 microns or the mean particle size is between 106 and 250 microns.

In a preferred embodiment, the compound of formula I in the eleventh aspect of the invention is:

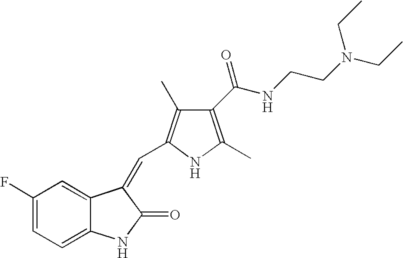

or a malate salt thereof.

In a preferred embodiment, the formulation of the eleventh aspect of the invention comprises 15-40% of the indolinone compound.

In a preferred embodiment, the formulation of the eleventh aspect of the invention the formulation comprises 40% w/w of an indolinone of formula I, or a pharmaceutically acceptable salt thereof, 47.5% w/w mannitol, 6% w/w croscaramellose sodium, 5% w/w povidone and 1.5% w/w magnesium stearate.

In a preferred embodiment, the compound of formula I in the ninth aspect of the invention is:

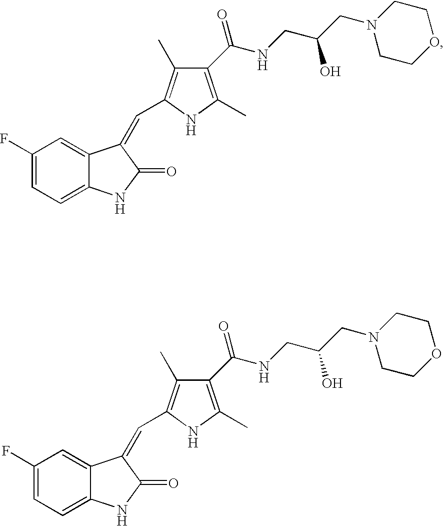

or mixtures thereof.

### DETAILED DESCRIPTION OF THE PREFERRED EMBODIMENTS

The instant invention features parenteral and oral formulations comprising an indolinone. In particular, the formulations aid the administration of indolinones to patients in need of treatment.

The indolinones of the preferred embodiments of the present invention may be formulated as any of the compositions and formulations described herein. Presently preferred formulations comprise an indolinone composition in a solid oral composition.

Definitions

The term “indolinone” as used herein includes pyrrole substituted 2-indolinones which, in addition to being otherwise optionally substituted on both the pyrrole and 2-indolinone portions of the compound, are necessarily substituted on the pyrrole moiety with the group —C(O)R¹⁰wherein R¹⁰ is —NR¹¹(CH₂)ₙR¹² or —NHCH₂CH(OH)CH₂R¹² wherein R¹¹ and R¹² are defined herein. Also within the scope of this invention pharmaceutically active salts, prodrugs, derivatives, and analogs of the indolinones.

Unless otherwise stated the following terms used in the specification and claims have the meanings discussed below:

“Alkyl” refers to a saturated aliphatic hydrocarbon radical including straight chain and branched chain groups of 1 to 20 carbon atoms (whenever a numerical range; e.g. “1-20”, is stated herein, it means that the group, in this case the alkyl group, may contain 1 carbon atom, 2 carbon atoms, 3 carbon atoms, etc. up to and including 20 carbon atoms). Alkyl groups containing from 1 to 4 carbon atoms are refered to as lower alkyl groups. When said lower alkyl groups lack substituents, they are referred to as unsubstituted lower alkyl groups. More preferably, an alkyl group is a medium size alkyl having 1 to 10 carbon atoms e.g., methyl, ethyl, propyl, 2-propyl, n-butyl, iso-butyl, tert-butyl, pentyl, and the like. Most preferably, it is a lower alkyl having 1 to 4 carbon atoms e.g., methyl, ethyl, propyl, 2-propyl, n-butyl, iso-butyl, or tert-butyl, and the like. The alkyl group may be substituted or unsubstituted. When substituted, the substituent group(s) is preferably one or more, more preferably one to three, even more preferably one or two substituent(s) independently selected from the group consisting of halo, hydroxy, unsubstituted lower alkoxy, aryl optionally substituted with one or more groups, preferably one, two or three groups which are independently of each other halo, hydroxy, unsubstituted lower alkyl or unsubstituted lower alkoxy groups, aryloxy optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 5- or 6-member heteroalicyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo , hydroxy, unsubstituted lower alkyl or unsubstituted lower alkoxy groups, mercapto, (unsubstituted lower alkyl)thio, arylthio optionally substituted with one or more groups, preferably one, two or three groups which are independently of each other halo, hydroxy, unsubstituted lower alkyl or unsubstituted lower alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, R¹⁸S(O)—, R¹⁸S(O)₂—, —C(O)OR¹⁸, R¹⁸C(O)O—, and —NR¹⁸R¹⁹, wherein R¹⁸ and R¹⁹ are independently selected from the group consisting of hydrogen, unsubstituted lower alkyl, trihalomethyl, unsubstituted (C₃-C₆)cycloalkyl, unsubstituted lower alkenyl, unsubstituted lower alkynyl and aryl optionally substituted with one or more, groups, preferably one, two or three groups which are independently of each other halo, hydroxy, unsubstituted lower alkyl or unsubstituted lower alkoxy groups.

Preferably, the alkyl group is substituted with one or two substituents independently selected from the group consisting of hydroxy, 5- or 6-member heteroalicyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, or —NR¹⁸R¹⁹, wherein R¹⁸ and R¹⁹ are independently selected from the group consisting of hydrogen, unsubstituted lower alkyl. Even more preferably the alkyl group is substituted with one or two substituents which are independently of each other hydroxy, dimethylamino, ethylamino, diethylamino, dipropylamino, pyrrolidino, piperidino, morpholino, piperazino, 4-lower alkylpiperazino, phenyl, imidazolyl, pyridinyl, pyridazinyl, pyrimidinyl, oxazolyl, triazinyl, and the like.

“Cycloalkyl” refers to a 3 to 8 member all-carbon monocyclic ring, an all-carbon 5-member/6-member or 6-member/6-member fused bicyclic ring or a multicyclic fused ring (a “fused” ring system means that each ring in the system shares an adjacent pair of carbon atoms with each other ring in the system) group wherein one or more of the rings may contain one or more double bonds but none of the rings has a completely conjugated pi—electron system.

Examples, without limitation, of cycloalkyl groups are cyclopropane, cyclobutane, cyclopentane, cyclopentene, cyclohexane, cyclohexadiene, adamantane, cycloheptane, cycloheptatriene, and the like. A cycloalkyl group may be substituted or unsubstituted. When substituted, the substituent group(s) is preferably one or more, more preferably one or two substituents, independently selected from the group consisting of unsubstituted lower alkyl, trihaloalkyl, halo, hydroxy, unsubstituted lower alkoxy, aryl optionally substituted with one or more, preferably one or two groups independently of each other halo, hydroxy, unsubstituted lower alkyl or unsubstituted lower alkoxy groups, aryloxy optionally substituted with one or more, preferably one or two groups independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more, preferably one or two groups independently of each other halo, hydroxy, unsubstituted lower alkyl dr unsubstituted lower alkoxy groups, 5-member heteroaryl having from 1 to 3 heteroatoms selected from the group consisting of nitrogen, oxygen and sulfur, the carbon and nitrogen atoms of the group being optionally substituted with one or more, preferably one or two groups independently of each other halo, hydroxy, unsubstituted lower alkyl or unsubstituted lower alkoxy groups, 5- or 6-member heteroalicyclic group having from 1 to 3 heteroatoms selected from the group consisting of nitrogen, oxygen and sulfur, the carbon and nitogen (if present)atoms in the group being optionally substituted with one or more, preferably one or two groups independently of each other halo, hydroxy, unsubstituted lower alkyl or unsubstituted lower alkoxy groups, mercapto, (unsubstituted lower alkyl)thio, arylthio optionally substituted with one or more, preferably one or two groups independently of each other halo, hydroxy, unsubstituted lower alkyl or unsubstituted. lower alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, R¹⁸S(O)—, R¹⁸S(O)₂—, —C(O)OR¹⁸, R¹⁸C(O)O—, and —NR¹⁸R¹⁹ are as defined above.

“Alkenyl” refers to a lower alkyl group, as defined herein, consisting of at least two carbon atoms and at least one carbon—carbon double bond. Representative examples include, but are not limited to, ethenyl, 1-propenyl, 2-propenyl, 1-, 2-, or 3-butenyl, and the like.

“Alkynyl” refers to a lower alkyl group, as defined herein, consisting of at least two carbon atoms and at least one carbon-carbon triple bond. Representative examples include, but are not limited to, ethynyl, 1-propynyl, 2-propynyl, 1-, 2-, or 3-butynyl, and the like.

“Aryl” refers to an all-carbon monocyclic or fused-ring polycyclic (i.e., rings which share adjacent pairs of carbon atoms) groups of 1 to 12 carbon atoms having a completely conjugated pi—electron system. Examples, without limitation, of aryl groups are phenyl, naphthalenyl and anthracenyl. The aryl group may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto, (unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, R¹⁸S(O)—, R¹⁸S(O)₂—, —C(O)OR¹⁸, R¹⁸C(O)O—, and —NR¹⁸R¹⁹, with R¹⁸ and R¹⁹ as defined above. Preferably, the aryl group is optionally substituted with one or two substituents independently selected from halo, unsubstituted lower alkyl, trihaloalkyl, hydroxy, mercapto, cyano, N—amido, mono or dialkylamino, carboxy, or N-sulfonamido.

“Heteroaryl” refers to a monocyclic or fused ring (i.e., rings which share an adjacent pair of atoms) group of 5 to 12 ring atoms containing one, two, or three ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine and carbazole. The heteroaryl group may be substituted or unsubstituted. When substituted, the substituted group(s) is preferably one or more, more preferably one, two, or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, R¹⁸S(O)—, R¹⁸O)₂—, —C(O)OR¹⁸, R¹⁸C(O)O—, and —NR¹⁸R¹⁹, with R¹⁸ and R¹⁹ as defined above. Preferably, the heteroaryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heteroalicyclic” refers to a monocyclic or fused ring group having in the ring(s) of 5 to 9 ring atoms in which one or two ring atoms are heteroatoms selected from N, O, or S(O)ₙ (where n is an integer from 0 to 2), the remaining ring atoms being C. The rings may also have one or more double bonds. However, the rings do not have a completely conjugated pi-electron system. Examples, without limitation, of unsubstituted heteroalicyclic groups are pyrrolidino, piperidino, piperazino, morpholino, thiomorpholino, homopiperazino, and the like. The heteroalicyclic ring may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, R¹⁸S(O)—, R¹⁸S(O)₂—, —C(O)OR¹⁸, R¹⁸C(O)O—, and —NR¹⁸R¹⁹, with R¹⁸ and R¹⁹ as defined above. Preferably, the heteroalicyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

Preferably, the heteroalicyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heterocycle” or “heterocyclo” means a saturated cyclic radical of 3 to 8 ring atoms in which one or two ring atoms are heteroatoms selected from N, O, or S(O)ₙ (where n is an integer from 0 to 2), the remaining ring atoms being C, where one or two C atoms may optionally be replaced by a carbonyl group. The heterocyclyl ring may be optionally substituted independently with one, two, or three substituents selected from optionally substituted lower alkyl (substituted with 1 or 2 substituents independently selected from carboxy or ester), haloalkyl, cyanoalkyl, halo, nitro, cyano, hydroxy, alkoxy, amino, monoalkylamino, dialkylamino, aralkyl, heteroaralkyl, —COR (where R is alkyl) or —COOR where R is (hydrogen or alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, 3-pyrrolidino, morpholino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof. Preferably, the heterocycle group is optionally substituted with one or two substituents independently selected from halo, unsubstituted lower alkyl, lower alkyl substituted with carboxy, ester hydroxy, mono or dialkylamino.

“Hydroxy” refers to an —OH group.

“Alkoxy” refers to both an —O-(unsubstituted alkyl) and an —O-(unsubstituted cycloalkyl) group. Representative examples include, but are not limited to, e.g., methoxy, ethoxy, propoxy, butoxy, cyclopropyloxy, cyclobutyloxy, cyclopentyloxy, cyclohexyloxy, and the like.

“Aryloxy” refers to both an —O-aryl and an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

“Mercapto” refers to an —SH group.

“Alkylthio” refers to both an —S-(unsubstituted alkyl) and an —S-(unsubstituted cycloalkyl) group. Representative examples include, but are not limited to, e.g., methylthio, ethylthio, propylthio, butylthio, cyclopropylthio, cyclobutylthio, cyclopentylthio, cyclohexylthio, and the like.

“Arylthio” refers to both an —S-aryl and an

—S-heteroaryl group, as defined herein. Representative examples include, but are not limited to, phenylthio, pyridinylthio, furanylthio, thientylthio, pyrimidinylthio, and the like and derivatives thereof.

“Acyl” refers to a —C(O)—R″ group, where R″ is selected from, the group consisting of hydrogen, unsubstituted lower alkyl, trihalomethyl, unsubstituted cycloalkyl, aryl optionally substituted with one or more, preferably one, two, or three substituents selected from the group consisting of unsubstituted lower alkyl, trihalomethyl, unsubstituted lower alkoxy, halo and —NR¹⁸R¹⁹ groups, heteroaryl (bonded through a ring carbon) optionally substituted with one or more, preferably one, two, or three substitutents selected from the group consisting of unsubstituted lower alkyl, trihaloalkyl, unsubstituted lower alkoxy, halo and —NR¹⁸R¹⁹ groups and heteroalicyclic (bonded through a ring carbon) optionally substituted with one or more, preferably one, two, or three substituents selected from the group consisting of unsubstituted lower alkyl, trihaloalkyl, unsubstituted lower alkoxy, halo and —NR¹⁸R¹⁹ groups. Representative acy groups include, but are not limited to, acetyl, trifluoroacetyl, benzoyl, and the like

“Aldehyde” refers to an acyl group in which R″ is hydrogen.

“Thioacyl” refers to a —C(S)—R″ group, with R″ as defined herein.

“Ester” refers to a —C(O)O—R″ group with R″ as defined herein except that R″ cannot be hydrogen.

“Acetyl” group refers to a —C(O)CH₃ group.

“Halo” group refers to fluorine, chlorine, bromine or iodine, preferably fluorine or chlorine.

“Trihalomethyl” group refers to a —CX₃ group wherein X is a halo group as defined herein.

“Trihalomethanesulfonyl” group refers to a X₃CS(═O)₂— groups with X as defined above.

“Cyano” refers to a —C≡N group.

“Methylenedioxy” refers to a —OCH₂O— group where the two oxygen atoms are bonded to adjacent carbon atoms.

“Ethylenedioxy” group refers to a —OCH₂CH₂O— where the two oxygen atoms are bonded to adjacent carbon atoms.

“S-sulfonamido” refers to a —S(O)₂NR¹⁸R¹⁹ group, with R¹⁸ and R¹⁹ as defined herein.

“N-sulfonamido” refers to a —NR¹⁸S(O)₂R¹⁹ group, with R¹⁸ and R¹⁹ as defined herein.

“O-carbamyl” group refers to a —OC(O)NR¹⁸R¹⁹ group with R¹⁸ and R¹⁹ as defined herein.

“N-carbamyl” refers to an R¹⁸OC(O)NR¹⁹-group, with R¹⁸ and R¹⁹ as defined herein.

“O-thiocarbamyl” refers to a —OC(S)NR¹⁸R¹⁹ group with R¹⁸ and R¹⁹ as defined herein.

“N-thiocarbamyl” refers to a R¹⁸OC(S)NR¹⁹-group, with R¹⁸ and R¹⁹ as defined herein.

“Amino” refers to an —NR¹⁸R¹⁹ group, wherein R¹⁸ and R¹⁹ are both hydrogen.

“C-amido” refers to a —C(O)NR¹⁸R¹⁹ group with R¹⁸ and R¹⁹ as defined herein.

“N-amido” refers to a R¹⁸C(O)NR¹⁹-group, with R¹⁸ and R¹⁹ as defined herein.

“Nitro” refers to a —NO₂ group.

“Haloalkyl” means an unsubstituted alkyl, preferably unsubstituted lower alkyl as defined above that is substituted with one or more same or different halo atoms, e.g., —CH₂Cl, —CF₃, —CH₂CF₃, —CH₂CCl₃, and the like.

“Aralkyl” means unsubstituted alkyl, preferably unsubstituted lower alkyl as defined above which is substituted with an aryl group as defined above, e.g., —CH₂phenyl, —(CH₂)₂phenyl, —(CH₂)₃phenyl, CH₃CH(CH₃)CH₂phenyl, and the like and derivatives thereof.

“Heteroaralkyl” group means unsubstituted alkyl, preferably unsubstituted lower alkyl as defined above which is substituted with a heteroaryl group, e.g., —CH₂pyridinyl, —(CH₂)₂pyrimidinyl, —(CH₂)₃imidazolyl, and the like, and derivatives thereof.

“Monoalkylamino” means a radical —NHR where R is an unsubstitued alkyl or unsubstituted cycloalkyl group as defined above, e.g., methylamino, (1-methylethyl)amino, cyclohexylamino, and the like.

“Dialkylamino” means a radical —NRR where each R is independently an unsubstitued alkyl or unsubstituted cycloalkyl group as defined above, e.g., dimethylamino, diethylamino, (1-methylethyl)-ethylamino, cyclohexylmethylamino, cyclopentylmethylamino, and the like.

“Cyanoalkyl” means unsubstituted alkyl, preferably unsubstituted lower alkyl as defined above, which is substituted with 1 or 2 cyano groups.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

The terms “2-indolinone”, “indolin-2-one” and “2-oxindole” are sometimes used interchangeably herein to refer to a molecule having the chemical structure:

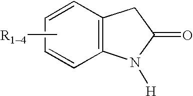

The term “pyrrole” refers to a molecule having the chemical structure:

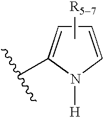

The term “pyrrole substituted 2-indolinone” and “3-pyrrolidenyl-2-indolinone” are used interchangeably herein to refer to a chemical compound having the general structure shown in Formula (I).

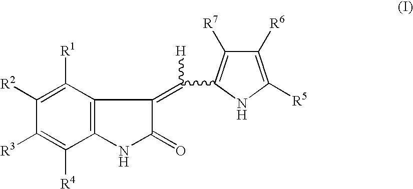

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R— and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)— or (S)-stereoisomers or as mixtures thereof. For example, if an R substituent in a compound of formula (I) is 2-hydroxyethyl, then the carbon to which the hydroxy group is attached is an asymmetric center and therefore the compound of Formula (I) can exist as an (R)— or (S)-stereoisomer. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

The compounds of Formula (I) may exhibit the phenomena of tautomerism and structural isomerism. For example, the compounds described herein may adopt an E or a Z configuration about the double bond connecting the 2-indolinone moiety to the pyrrole moiety or they may be a mixture of E and Z. This invention encompasses any tautomeric or structural isomeric form and mixtures thereof which possess the ability to modulate RTK, CTK and/or STK activity and is not limited to any one tautomeric or structural isomeric form.

The compounds of this invention, as well as the precursor 2-oxindoles and aldehydes, may be readily synthesized using techniques well known in the chemical arts. The syntheses of the compounds of the preferred embodiments of the present invention is disclosed in U.S. Ser. No. 10/076,140, filed Feb. 15, 2002, PCT Application No. PCT/US02/04407, and published PCT application WO 01/60814; and U.S. Ser. No. 10/281,985, filed Aug. 13, 2002, claiming priority to U.S. Serial No. 60/312,353, filed Aug. 15, 2001; all of which are incorporated herein by reference. Yet, it will be appreciated by those skilled in the art that other synthetic pathways for forming the compounds of the invention are available and that the following is offered by way of example and not limitation.

Preferred indolinones include:

| | |
|---|---|
| 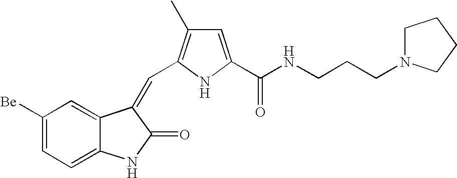 | 5-(5-Bromo-2-oxo-1,2-dihydro- indol-3-ylidenemethyl)-4-methyl-1H-pyrrole-2-car- boxylic acid(3-pyrrolidin-1-ylpropyl)amide |
| 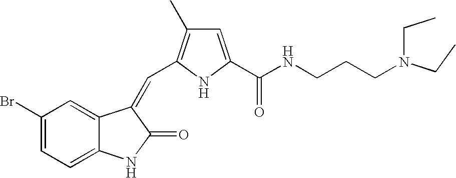 | 5-(5-Bromo-2-oxo-1,2-dihydro- indol-3-ylidenemethyl)-4-methyl-1H-pyrrole-2-car- boxylic acid(3-diethylaminopropyl)amide |
| 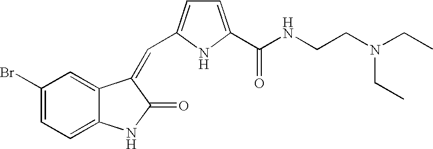 | 5-(5-Bromo-2-oxo-1,2-dihydro- indol-3-ylidenemethyl)-1H-pyrrole-2-car- boxylic acid(2-diethylaminoethyl)amide |
| 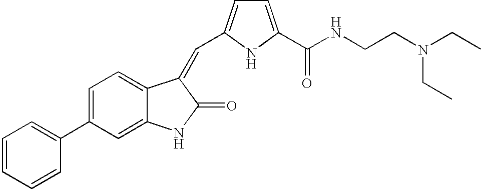 | 5-(2-Oxo-6-phenyl-1,2-dihydro- indol-3-ylidenemethyl)-1H-pyrrole-2-car- boxylic acid(2-diethylaminoethyl)amide |
| 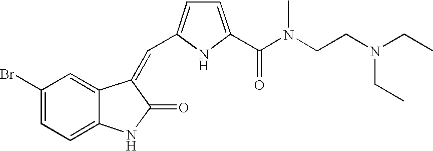 | 5-(5-Bromo-2-oxo-1,2-dihydro- indol-3-ylidenemethyl)-1H-pyrrole-2-car- boxylic acid(2-diethylaminoethyl)methylamide |
| 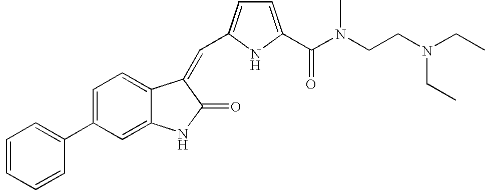 | 5-(2-Oxo-6-phenyl-1,2-dihydro- indol-3-ylidenemethyl)-1H-pyrrole-2-car- boxylic acid(2-diethylaminoethyl)methylamide |
| 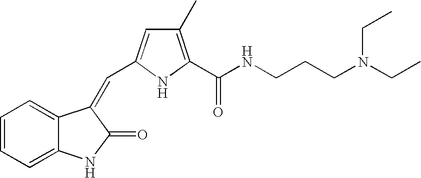 | 3-Methyl-5-(2-oxo-1,2-dihydro- indol-3-ylidenemethyl)-1H-pyrrole-2-car- boxylic acid(3-diethylaminopropyl)amide |
| 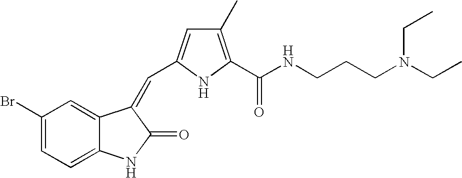 | 5-(5-Bromo-2-oxo-1,2-dihydro- indol-3-ylidenemethyl)-3-methyl-1H-pyrrole-2-car- boxylic acid(3-diethylaminopropyl)amide |
| 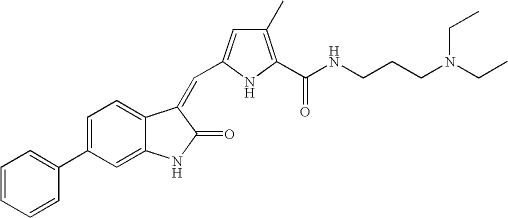 | 3-Methyl-5-(2-oxo-6-phenyl-1,2-dihydro- indol-3-ylidenemethyl)-1H-pyrrole-2-car- boxylic acid(3-diethylaminopropyl)amide |
| 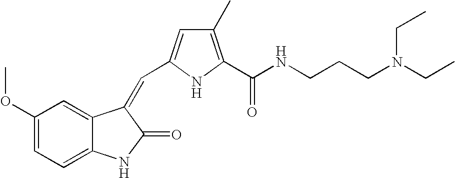 | 5-(5-Methoxy-2-oxo-1,2-dihydro- indol-3-ylidenemethyl)-3-methyl-1H-pyrrole-2-car- boxylic acid(3-diethylaminopropyl)amide |
| 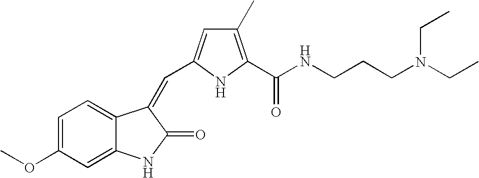 | 5-(6-Methoxy-2-oxo-1,2-dihydro- indol-3-ylidenemethyl)-3-methyl-1H-pyrrole-2-car- boxylic acid(3-diethylaminopropyl)amide |
| 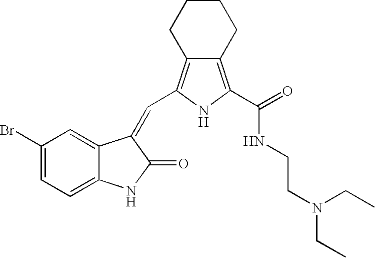 | 3-(5-Bromo-2-oxo-1,2-dihydro- indol-3-ylidenemethyl)-4,5,6,7-tetrahydro-2H-iso- indole-1-carboxylic acid(2-diethylaminoethyl)amide |
| 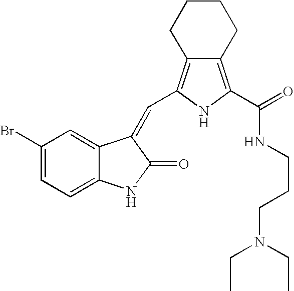 | 3-(5-Bromo-2-oxo-1,2-dihydro- indol-3-ylidenemethyl)-4,5,6,7-tetrahydro-2H-iso- indole-1-carboxylic acid(3-diethylaminopropyl)amide |
| 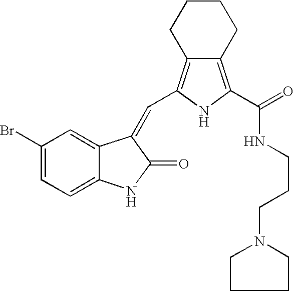 | 3-(5-Bromo-2-oxo-1,2-dihydro- indol-3-ylidenemethyl)-4,5,6,7-tetrahydro-2H-iso- indole-1-carboxylic acid(3-pyrrolidin-1-ylpropyl)amide |
| 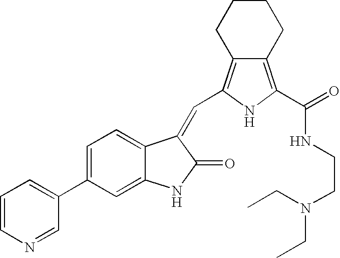 | 3-(2-Oxo-6-pyridin-3-yl-1,2-dihydro- indol-3-ylidenemethyl)-4,5,6,7-tetra- hydro-2H-isoindole-1-carboxylic acid(2-diethylaminoethyl)amide |
| 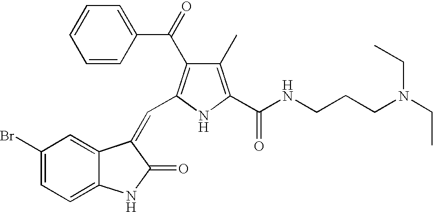 | 4-Benzoyl-5-(5-bromo-2-oxo-1,2-dihydro- indol-3-ylidenemethyl)-3-methyl-1H-pyr- role-2-carboxylic acid(3-diethylaminopropyl)amide |
| 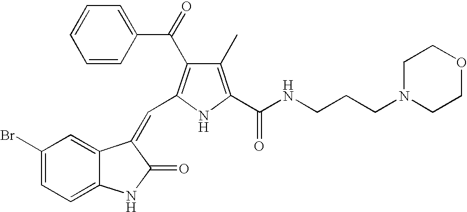 | 4-Benzoyl-5-(5-bromo-2-oxo-1,2-dihydro- indol-3-ylidenemethyl)-3-methyl-1H-pyr- role-2-carboxylic acid(3-morpholin-4-ylpropyl)amide |
| 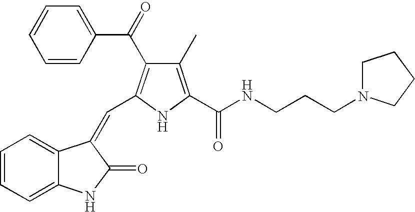 | 4-Benzoyl-3-methyl-5-(2-oxo-1,2-dihydro- indol-3-ylidenemethyl)-1H-pyrrole-2-car- boxylic acid(3-pyrrolidin-1-ylpropyl)amide |
| 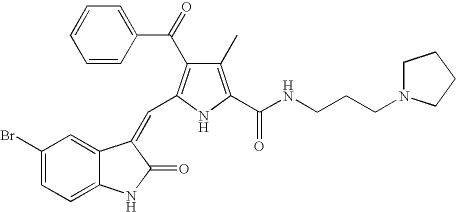 | 4-Benzoyl-5-(5-bromo-2-oxo-1,2-dihydro- indol-3-ylidenemethyl)-3-methyl-1H-pyr- role-2-carboxylic acid(3-pyrrolidin-1-ylpropyl)amide |
| 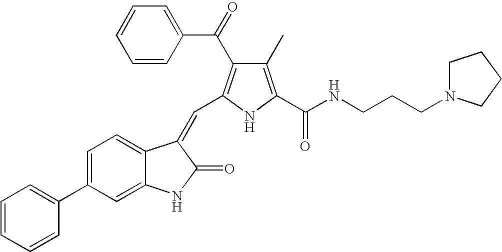 | 4-Benzoyl-3-methyl-5-(2-oxo-6-phenyl-1,2-dihydro- indol-3-ylidenemethyl)-1H-pyr- role-2-carboxylic acid(3-pyrrolidin-1-ylpropyl)amide |
| 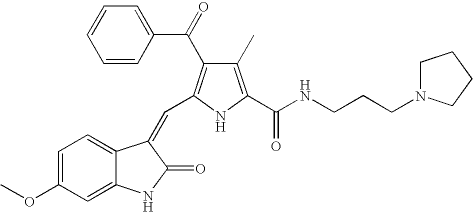 | 4-Benzoyl-5-(6-methoxy-2-oxo-1,2-dihydro- indol-3-ylidenemethyl)-3-methyl-1H-pyr- role-2-carboxylic acid(3-pyrrolidin-1-ylpropyl)amide |
| 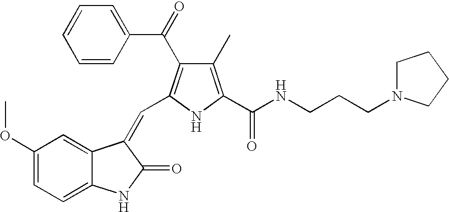 | 4-Benzoyl-5-(5-methoxy-2-oxo-1,2-dihydro- indol-3-ylidenemethyl)-3-methyl-1H-pyr- role-2-carboxylic acid(3-pyrrolidin-1-ylpropyl)amide |
| 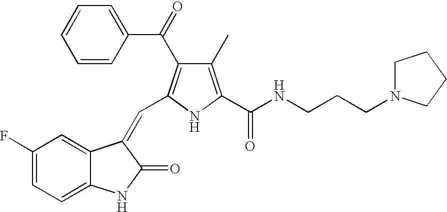 | 4-Benzoyl-5-(5-fluoro-2-oxo-1,2-dihydro- indol-3-ylidenemethyl)-3-methyl-1H-pyr- role-2-carboxylic acid(3-pyrrolidin-1-ylpropyl)amide |
| 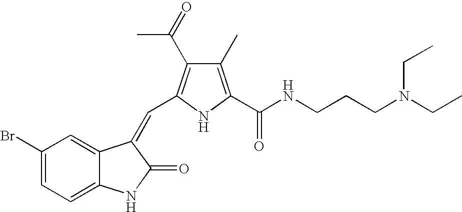 | 4-Acetyl-5-(5-bromo-2-oxo-1,2-dihydro- indol-3-ylidenemethyl)-3-methyl-1H-pyr- role-2-carboxylic acid(3-diethylaminopropyl)amide |
| 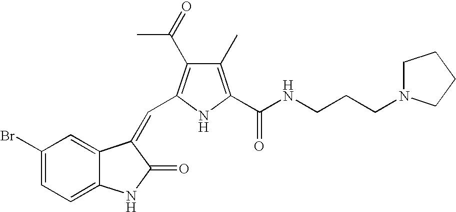 | 4-Acetyl-5-(5-bromo-2-oxo-1,2-dihydro- indol-3-ylidenemethyl)-3-methyl-1H-pyr- role-2-carboxylic acid(3-pyrrolidin-1-ylpropyl)amide |
| 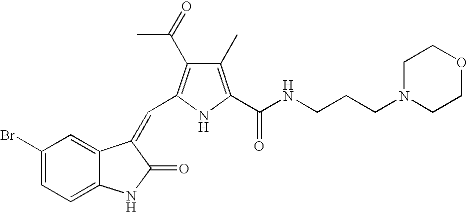 | 4-Acetyl-5-(5-bromo-2-oxo-1,2-dihydro- indol-3-ylidenemethyl)-3-methyl-1H-pyr- role-2-carboxylic acid(3-morpholin-4-ylpropyl)amide |
| 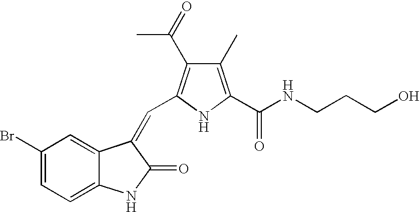 | 4-Acetyl-5-(5-bromo-2-oxo-1,2-dihydro- indol-3-ylidenemethyl)-3-methyl-1H-pyr- role-2-carboxylic acid(3-hydroxy-propyl)amide |
| 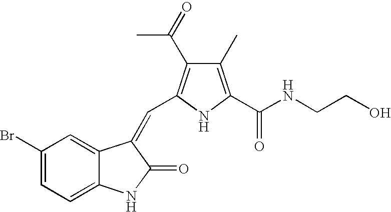 | 4-Acetyl-5-(5-bromo-2-oxo-1,2-dihydro- indol-3-ylidenemethyl)-3-methyl-1H-pyr- role-2-carboxylic acid(2-hydroxy-ethyl)amide |
| 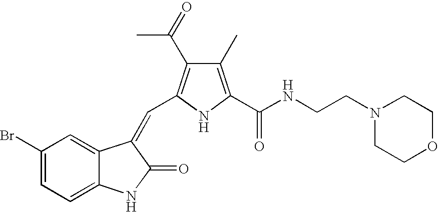 | 4-Acetyl-5-(5-bromo-2-oxo-1,2-dihydro- indol-3-ylidenemethyl)-3-methyl-1H-pyr- role-2-carboxylic acid(2-morpholin-4-yl-ethyl)amide |
| 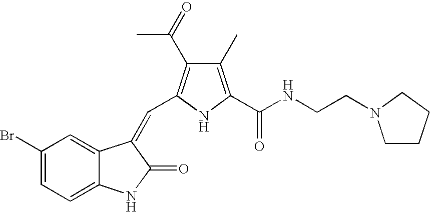 | 4-Acetyl-5-(5-bromo-2-oxo-1,2-dihydro- indol-3-ylidenemethyl)-3-methyl-1H-pyr- role-2-carboxylic acid(2-pyrrolidin-1-yl-ethyl)amide |
| 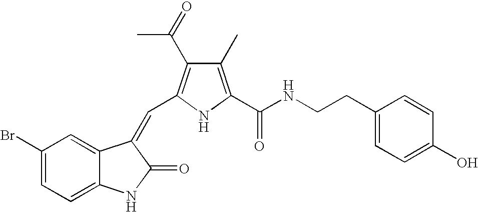 | 4-Acetyl-5-(5-bromo-2-oxo-1,2-dihydro- indol-3-ylidenemethyl)-3-methyl-1H-pyr- role-2-carboxylic acid[2-(4-hydroxy-phenyl)-ethyl]amide |
| 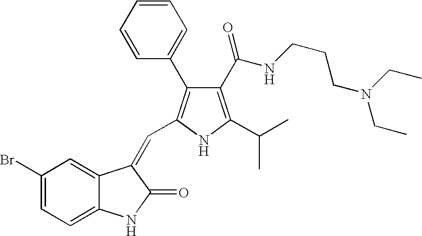 | 5-(5-Bromo-2-oxo-1,2-dihydro- indol-3-ylidenemethyl)-2-isopropyl-4-phenyl-1H-pyr- role-3-carboxylic acid(3-diethylaminopropyl)amide |
| 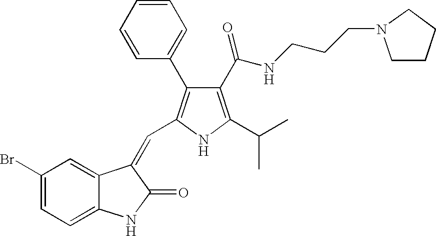 | 5-(5-Bromo-2-oxo-1,2-dihydro- indol-3-ylidenemethyl)-2-isopropyl-4-phenyl-1H-pyr- role-3-carboxylic acid(3-pyrrolidin-1-ylpropyl)amide |
| 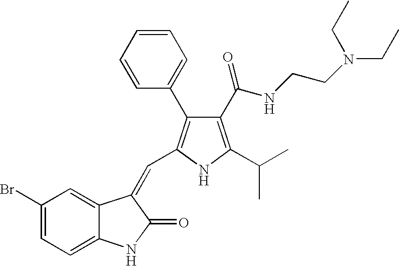 | 5-(5-Bromo-2-oxo-1,2-dihydro- indol-3-ylidenemethyl)-2-isopropyl-4-phenyl-1H-pyr- role-3-carboxylic acid(2-diethylaminoethyl)amide |
| 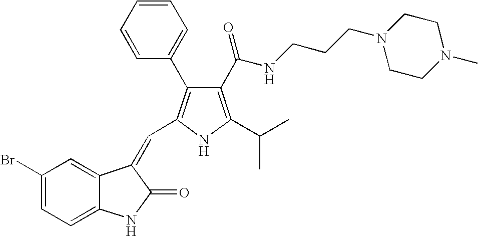 | 5-(5-Bromo-2-oxo-1,2-dihydro- indol-3-ylidenemethyl)-2-isopropyl-4-phenyl-1H-pyr- role-3-carboxylic acid[3-(4-methyl-piperazin-1-yl)-propyl]amide |
| 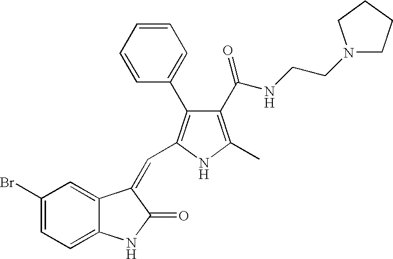 | 5-(5-Bromo-2-oxo-1,2-dihydro- indol-3-ylidenemethyl)-2-methyl-4-phenyl-1H-pyr- role-3-carboxylic acid(2-pyrrolidin-1-yl-ethyl)amide |
| 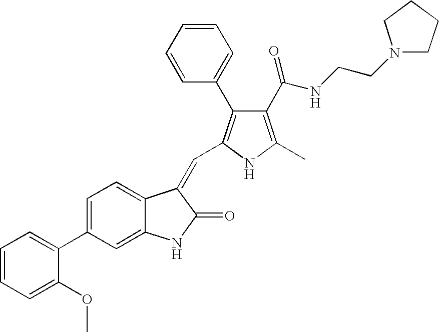 | 5-[6-(2-Methoxy-phenyl)-2-oxo-1,2-dihydro- indol-3-ylidenemethyl]-2-methyl-4-phenyl-1H-pyr- role-3-carboxylic acid(2-pyrrolidin-1-yl-ethyl)amide |
| 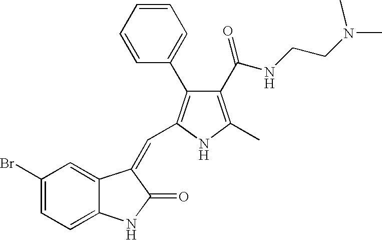 | 5-(5-Bromo-2-oxo-1,2-dihydro- indol-3-ylidenemethyl)-2-methyl-4-phenyl-1H-pyr- role-3-carboxylic acid(2-dimethylamino-ethyl)amide |
| 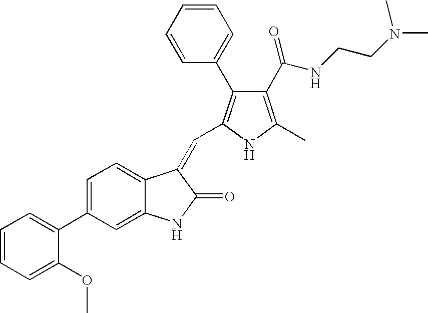 | 5-[6-(2-Methoxy-phenyl)-2-oxo-1,2-dihydro- indol-3-ylidenemethyl]-2-methyl-4-phe- nyl-1H-pyrrole-3-carboxylic acid(2-dimethylamino-ethyl)amide |
| 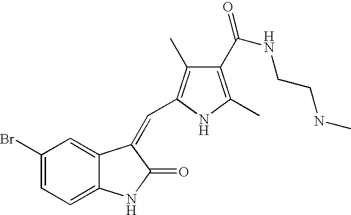 | 5-(5-Bromo-2-oxo-1,2-dihydro- indol-3-ylidenemethyl)-2,4-dimethyl-1H-pyr- role-3-carboxylic acid(2-dimethylamino-ethyl)amide |
| 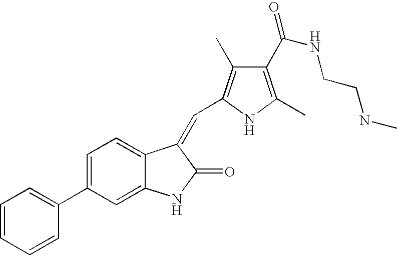 | 2,4-Dimethyl-5-(2-oxo-6-phenyl-1,2-dihydro- indol-3-ylidenemethyl)-1H-pyr- role-3-carboxylic acid(2-dimethylamino-ethyl)amide |
| 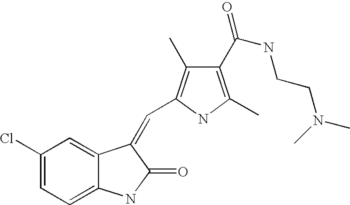 | 5-(5-Chloro-2-oxo-1,2-dihydro- indol-3-ylidenemethyl)-2,4-dimethyl-1H-pyr- role-3-carboxylic acid(2-dimethylamino-ethyl)amide |
| 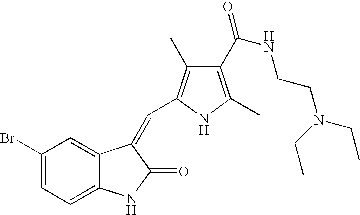 | 5-(5-Bromo-2-oxo-1,2-dihydro- indol-3-ylidenemethyl)-2,4-dimethyl-1H-pyr- role-3-carboxylic acid(2-diethylaminoethyl)amide |
| 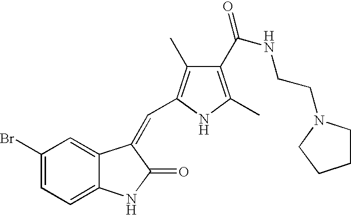 | 5-(5-Bromo-2-oxo-1,2-dihydro- indol-3-ylidenemethyl)-2,4-dimethyl-1H-pyr- role-3-carboxylic acid(2-pyrrolidin-1-yl-ethyl)amide |
| 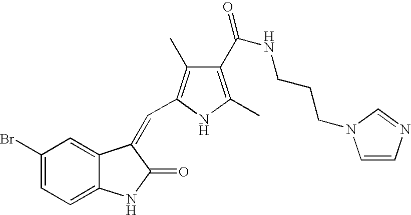 | 5-(5-Bromo-2-oxo-1,2-dihydro- indol-3-ylidenemethyl)-2,4-dimethyl-1H-pyr- role-3-carboxylic acid(3-imidazol-1-ylpropyl)amide |
| 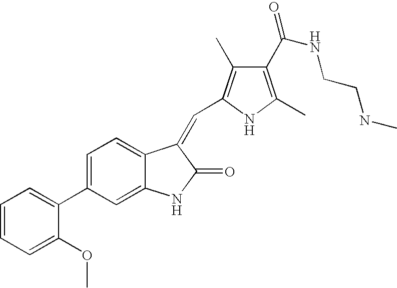 | 5-[6-(2-Methoxy-phenyl)-2-oxo-1,2-di- hydroindol-3-ylidenemethyl]-2,4-di- methyl-1H-pyrrole-3-carboxylic acid(2-dimethylamino-ethyl)amide |
| 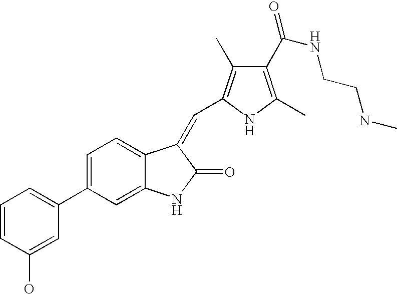 | 5-[6-(3-Methoxy-phenyl)-2-oxo-1,2-di- hydroindol-3-ylidenemethyl]-2,4-di- methyl-1H-pyrrole-3-carboxylic acid(2-dimethylamino-ethyl)amide |
| 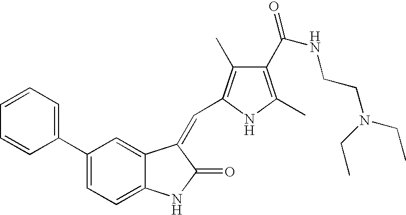 | 2,4-Dimethyl-5-(2-oxo-5-phenyl-1,2-dihydro- indol-3-ylidenemethyl)-1H-pyr- role-3-carboxylic acid(2-diethylaminoethyl)amide |
| 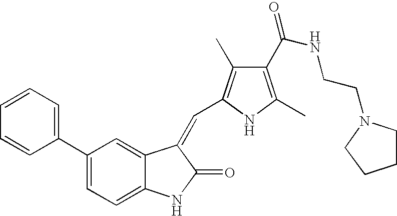 | 2,4-Dimethyl-5-(2-oxo-5-phenyl-1,2-dihydro- indol-3-ylidenemethyl)-1H-pyr- role-3-carboxylic acid(2-pyrrolidin-1-yl-ethyl)amide |
| 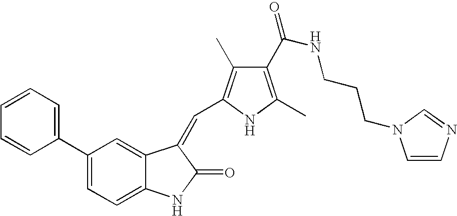 | 2,4-Dimethyl-5-(2-oxo-5-phenyl-1,2-dihydro- indol-3-ylidenemethyl)-1H-pyr- role-3-carboxylic acid(3-imidazol-1-ylpropyl)amide |
| 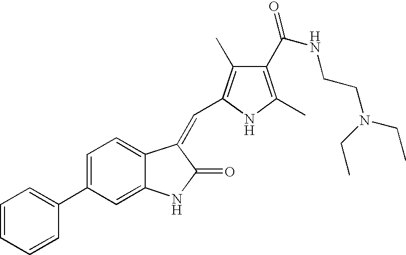 | 2,4-Dimethyl-5-(2-oxo-6-phenyl-1,2-dihydro- indol-3-ylidenemethyl)-1H-pyr- role-3-carboxylic acid(2-diethylaminoethyl)amide |
| 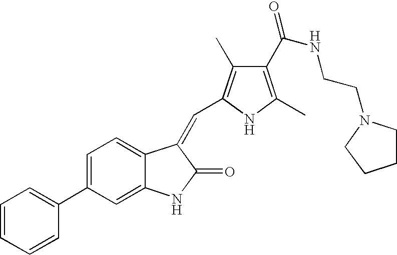 | 2,4-Dimethyl-5-(2-oxo-6-phenyl-1,2-dihydro- indol-3-ylidenemethyl)-1H-pyr- role-3-carboxylic acid(2-pyrrolidin-1-yl-ethyl)amide |
| 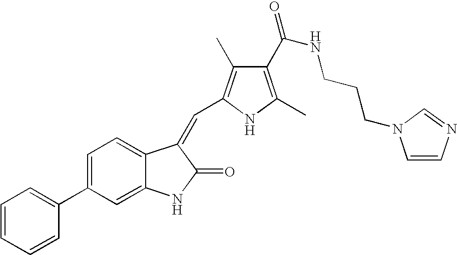 | 2,4-Dimethyl-5-(2-oxo-6-phenyl-1,2-dihydro- indol-3-ylidenemethyl)-1H-pyr- role-3-carboxylic acid(3-imidazol-1-ylpropyl)amide |
| 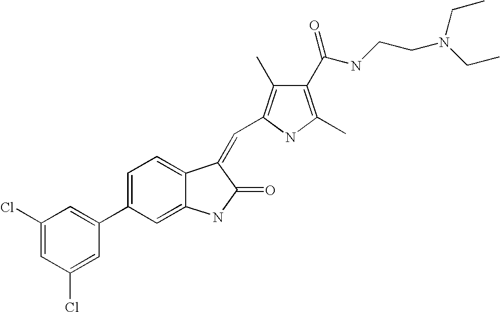 | 5-[6-(3,5-Dichloro-phe- nyl)-2-oxo-1,2-dihydroindole-3-ylidenemethyl]-2,4-di- methyl-1H-pyrrole-3-carboxylic acid(2-diethylaminoethyl)amide |
| 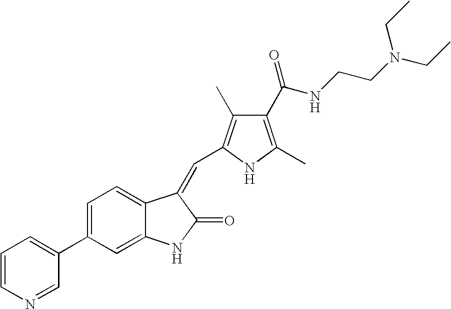 | 2,4-Dimethyl-5-(2-oxo-6-py- ridin-3-yl-1,2-dihydroindol-3-ylidenemethyl)-1H-pyr- role-3-carboxylic acid(2-diethylaminoethyl)amide |
| 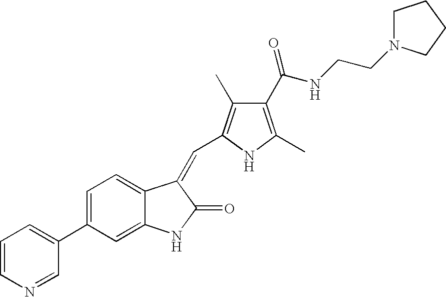 | 2,4-Dimethyl-5-(2-oxo-6-py- ridin-3-yl-1,2-dihydroindol-3-ylidenemethyl)-1H-pyr- role-3-carboxylic acid(2-pyrrolidin-1-yl-ethyl)amide |
| 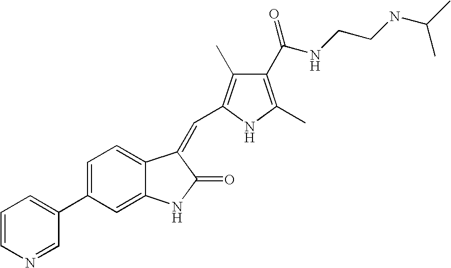 | 2,4-Dimethyl-5-(2-oxo-6-py- ridin-3-yl-1,2-dihydroindol-3-ylidenemethyl)-1H-pyr- role-3-carboxylic acid(3-dimethylamino-propyl)amide |
| 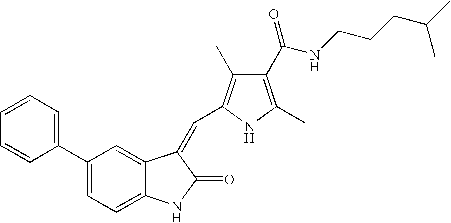 | 2,4-Dimethyl-5-(2-oxo-5-phenyl-1,2-di- hydroindol-3-ylidenemethyl)-1H-pyr- role-3-carboxylic acid(3-dimethylamino-propyl)amide |
| 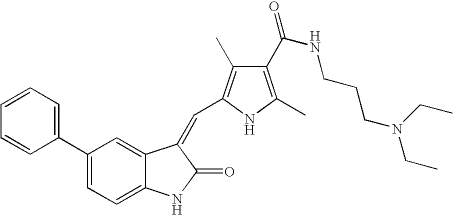 | 2,4-Dimethyl-5-(2-oxo-5-phenyl-1,2-di- hydroindol-3-ylidenemethyl)-1H-pyr- role-3-carboxylic acid(3-diethylaminopropyl)amide |
| 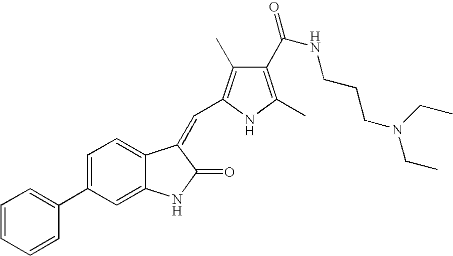 | 2,4-Dimethyl-5-(2-oxo-6-phenyl-1,2-di- hydroindol-3-ylidenemethyl)-1H-pyr- role-3-carboxylic acid(3-diethylaminoproyl)amide |
| 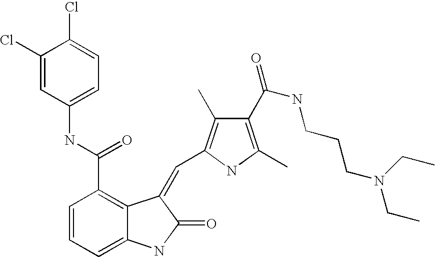 | 3-[4-(3-Diethylamino-propyl- carbamoyl)-3,5-dimethyl-1H-pyrrol-2-ylmeth- ylene]-2-oxo-2,3-dihydro-1H-in- dole-4-carboxylic acid(3-chloro-4-meth- oxy-phenyl)amide |
| 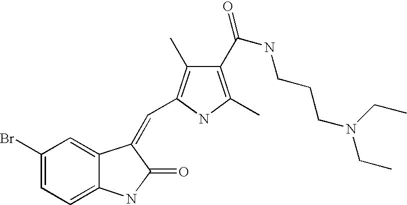 | 5-(5-Bromo-2-oxo-1,2-dihydro- indol-3-ylidenemethyl)-2,4-dimethyl-1H-pyr- role-3-carboxylic acid(3-diethylaminopropyl)amide |
| 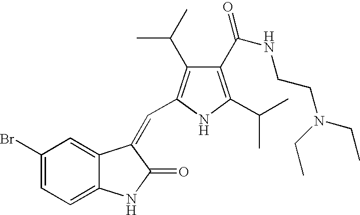 | 5-(5-Bromo-2-oxo-1,2-dihydro- indol-3-ylidenemethyl)-2,4-diisopropyl-1H-pyr- role-3-carboxylic acid(2-diethylaminoethyl)amide |
| 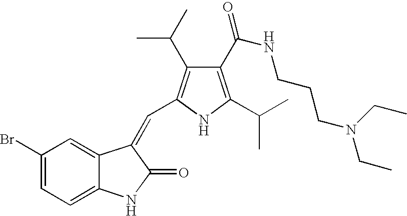 | 5-(5-Bromo-2-oxo-1,2-dihydro- indol-3-ylidenemethyl)-2,4-diisopropyl-1H-pyr- role-3-carboxylic acid(3-diethylaminopropyl)amide |
| 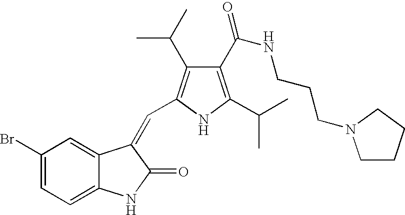 | 5-(5-Bromo-2-oxo-1,2-dihydro- indol-3-ylidenemethyl)-2,4-diisopropyl-1H-pyr- role-3-carboxylic acid(3-pyrrolidin-1-ylpropyl)amide |
| 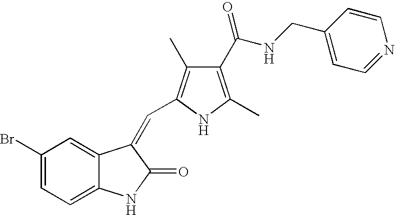 | 5-(5-Bromo-2-oxo-1,2-dihydro- indol-3-ylidenemethyl)-2,4-dimethyl-1H-pyr- role-3-carboxylic acid(pyridin-4-ylmethyl)amide |
| 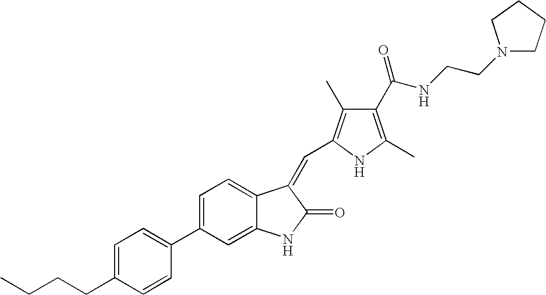 | 5-[6-(4-Butyl-phenyl)-2-oxo-1,2-di- hydroindol-3-ylidenemethyl]-2,4-dimethyl-1H-pyr- role-3-carboxylic acid(2-pyrrolidin-1-yl-ethyl)amide |
| 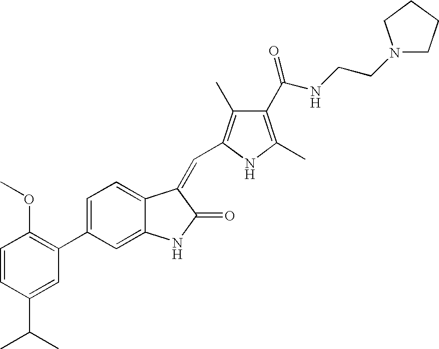 | 5-[6-(5-isopropyl-2-methoxy-phe- nyl)-2-oxo-1,2-dihydroindol-3-ylidene- methyl]-2,4-dimethyl-1H-pyrrole-3-carboxylic acid(2-pyrrolidin-1-yl-eth- yl)amide |
| 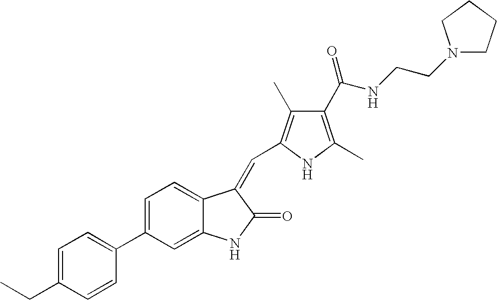 | 5-[6-(4-Ethyl-phenyl)-2-oxo-1,2-di- hydroindol-3-ylidenemethyl]-2,4-dimethyl-1H-pyr- role-3-carboxylic acid(2-pyrrolidin-1-yl-ethyl)amide |
| 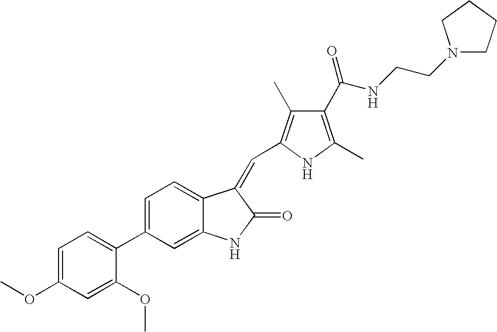 | 5-[6-(2,4-Dimethoxy-phenyl)-2-oxo-1,2-di- hydroindol-3-ylidenemethyl]-2,4-di- methyl-1H-pyrrole-3-carboxylic acid(2-pyrrolidin-1-yl-ethyl)amide |
| 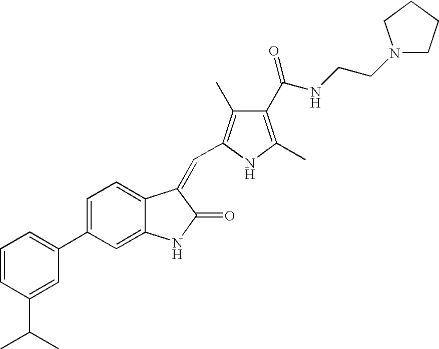 | 5-[6-(3-isopropyl-phenyl)-2-oxo-1,2-di- hydroindol-3-ylidenemethyl]-2,4-di- methyl-1H-pyrrole-3-carboxylic acid(2-pyrrolidin-1-yl-ethyl)amide |
| 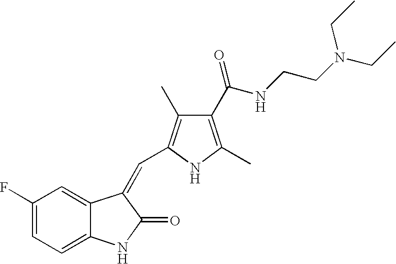 | 5-(5-Fluoro-2-oxo-1,2-dihydro- indol-3-ylidenemethyl)-2,4-dimethyl-1H-pyr- role-3-carboxylic acid(2-diethylaminoethyl)amide |
| 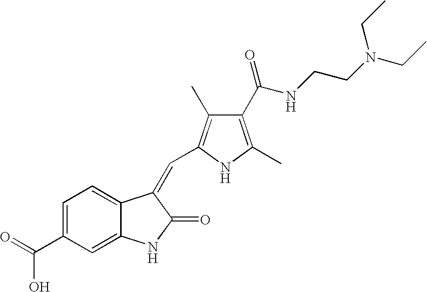 | 3-[4-(2-diethylamino- ethylcarbamoyl)-3,5-di- methyl-1H-pyrrol-2-ylmethylene]-2-oxo-2,3-di- hydro-1H-indole-6-carboxylic acid |
| 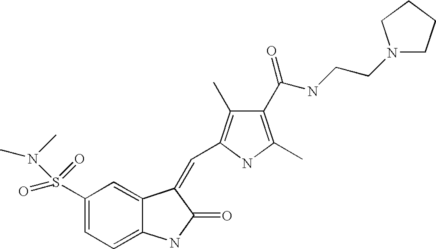 | 5-(5-Dimethylsulfamoyl-2-ooxo-1,2-di- hydroindol-3-ylidenemethyl)-2,4-di- methyl-1H-pyrrole-3-carboxylic acid(2-pyrrolidin-1-yl-ethyl)amide |
| 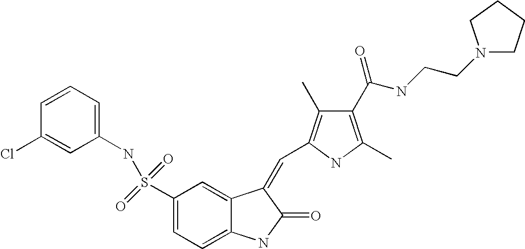 | 5-[5-(3-Chloro-phenylsulfa- moyl)-2-oxo-1,2-dihydroindol-3-ylidenemethyl]-2,4-di- methyl-1H-pyrrole-3-carboxylic acid(2-pyrrolidin-1-yl-ethyl)amide |
| 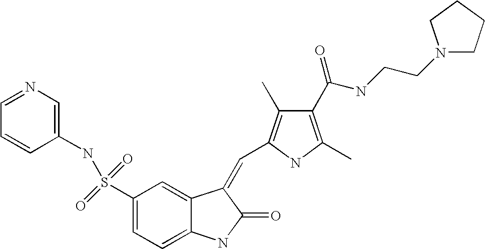 | 2,4-Dimethyl-5-[2-oxo-5-(py- ridin-3-ylsulfamoyl)-1,2-dihydroindl)-3-ylidene- methyl)-1H-pyrrole-3-carboxylic acid(2-pyrrolidin-1-yl-ethyl)amide |
| 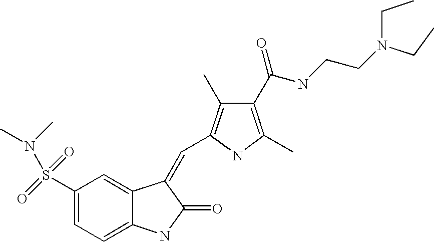 | 5-(5-Dimethylsulfamoyl-2-oxo-1,2-di- hydroindol-3-ylidenemethyl)-2,4-di- methyl-1H-pyrrole-3-carboxylic acid(2-diethylaminoethyl)amide |
| 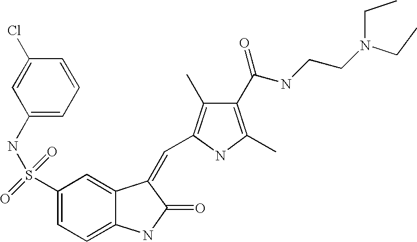 | 5-[5-(3-Chloro-phenylsulfa- moyl)-2-oxo-1,2-dihydroindol-3-ylidenemethyl]-2,4-di- methyl-1H-pyrrole-3-carboxylic acid(2-diethylaminoethyl)amide |
| 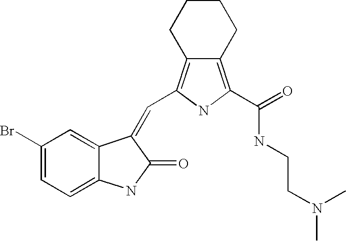 | 3-(5-Bromo-2-oxo-1,2-dihydro-in- dol-3-ylidenemethyl)-4,5,6,7-tetrahydro-2H-iso- indole-1-carboxylic acid(2-dimethylamino-ethyl)-amide |
| 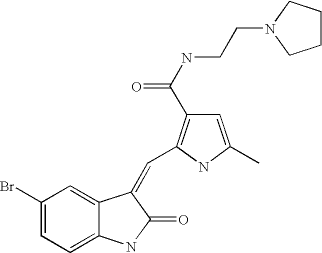 | 2-(5-Bromo-2-oxo-1,2-dihydro-in- dol-3-ylidenemethyl)-5-methyl-1H-pyrrole-3-car- boxylic acid(2-pyrrolidin-1-yl-ethyl)-amide |
| 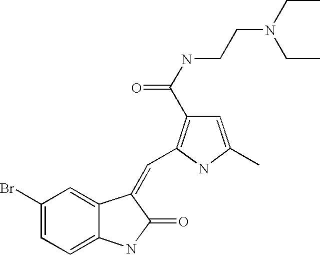 | 2-(5-Bromo-2-oxo-1,2-dihydro-in- dol-3-ylidenemethyl)-5-methyl-1H-pyrrole-3-car- boxylic acid(2-diethylamino-ethyl)-amide |
| 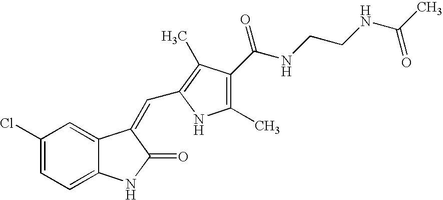 | 5-[5-Chloro-2-oxo-1,2-dihydro-in- dol-(3Z)-ylidene- methyl]-2,4-dimethyl-1H-pyr- role-3-carboxylic acid(2-acetyl- amino-ethyl)-amide |
| 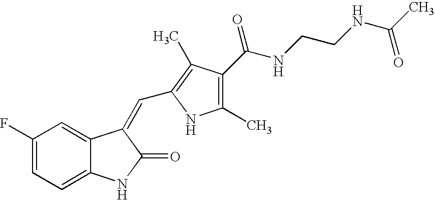 | 5-[5-Fluoro-2-oxo-1,2-dihydro-in- dol-(3Z)-ylidene- methyl]-2,4-dimethyl-1H-pyr- role-3-carboxylic acid(2-acetyl- amino-ethyl)-amide |
| 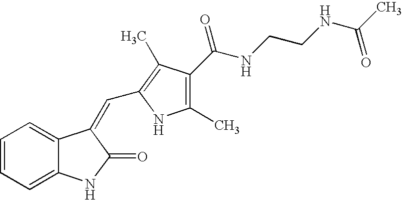 | 2,4-Dimethyl-5-[2-oxo-1,2-dihydro-in- dol-(3Z)-ylidenemethyl]-1H-pyrrole-3-car- boxylic acid(2-acetylamino-eth- yl)-amide |
| 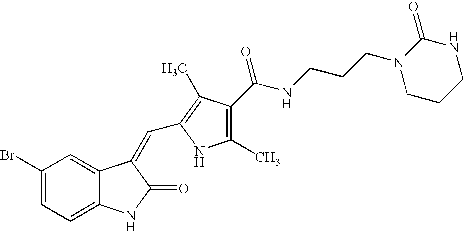 | 5-[5-Bromo-2-oxo-1,2-dihydro-indol-(3Z)-ylidene- methyl]-2,4-dimethyl-1H-pyr- role-3-carboxylic acid[3-(2-oxo-tetra- hydro-pyrimidin-1-yl)-propyl]-amide |
| 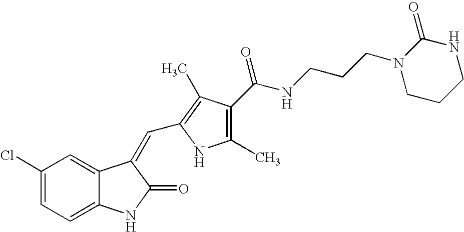 | 5-[5-Chloro-2-oxo-1,2-dihydro-indol-(3Z)-ylidene- methyl]-2,4-dimethyl-1H-pyr- role-3-carboxylic acid[3-(2-oxo-tetra- hydro-pyrimidin-1-yl)-propyl]-amide |
| 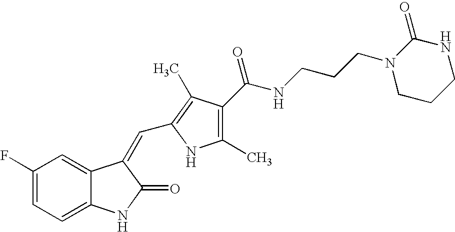 | 5-[5-Fluoro-2-oxo-1,2-dihydro-indol-(3Z)-ylidene- methyl]-2,4-dimethyl-1H-pyr- role-3-carboxylic acid[3-(2-oxo-tetra- hydro-pyrimidin-1-yl)-propyl]-amide |
| 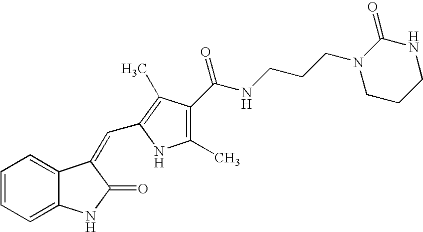 | 2,4-Dimethyl-5-[2-oxo-1,2-dihydro-in- dol-(3Z)-ylidenemethyl]-1H-pyrrole-3-car- boxylic acid[3-(2-oxo-tetra- hydro-pyrimidin-1-yl)-propyl]-amide |
| 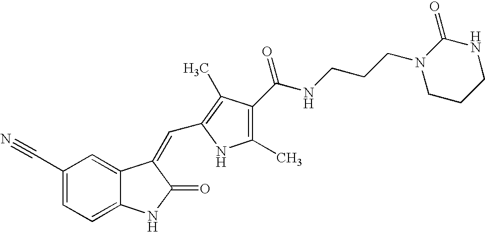 | 5-[5-Cyano-2-oxo-1,2-dihydro-indol-(3Z)-ylidene- methyl]-2,4-dimethyl-1H-pyr- role-3-carboxylic acid[3-(2-oxo-tetra- hydro-pyrimidin-1-yl)-propyl]-amide |
| 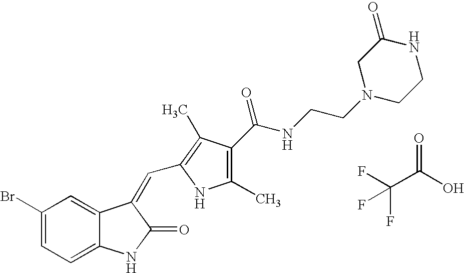 | Trifluoro-acetate4-[2-({5-[5-bromo-2-oxo-1,2-di- hydro-indol-(3Z)-ylidene- methyl]-2,4-dimethyl-1H-pyr- role-3-carbonyl}-amino)-ethyl]-2-oxo-pipe- razin-1-ium; |
| 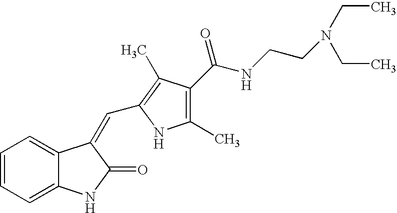 | 2,4-Dimethyl-5-[2-oxo-1,2-dihydro-indol-(3Z)-ylidene- methyl]-1H-pyrrole-3-carboxylic acid(2-diethylaminoethyl)-amide |
| 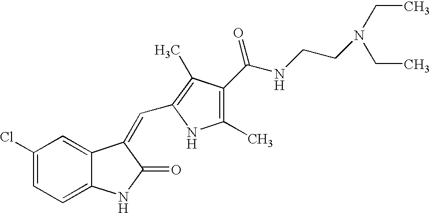 | 5-[5-Chloro-2-oxo-1,2-dihydro-indol-(3Z)-ylidene- methyl]-2,4-dimethyl-1H-pyrrole-3-carboxylic- acid(2-diethylaminoethyl)-amide |
| 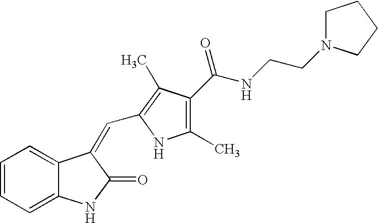 | 2,4-Dimethyl-5-[2-oxo-1,2-dihydro-indol-(3Z)-ylidene- methyl]-2,4-dimethyl-1H-pyrrole-3-car- boxylic acid(2-pyrrolidin-1-ylethyl)-amide |
| 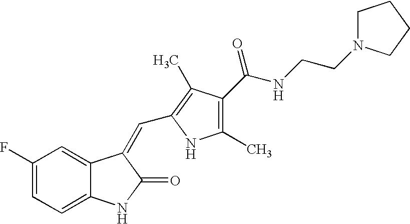 | 5-[5-Fluoro-2-oxo-1,2-dihydro-indol-(3Z)-ylidene- methyl]-2,4-dimethyl-1H-pyrrole-3-carboxylic acid(2-pyrrolidin-1-ylethyl)-amide |
| 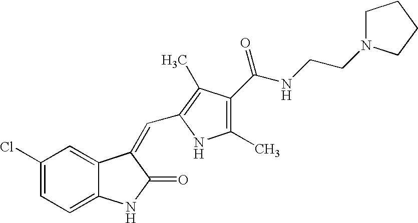 | 5-[5-Chloro-2-oxo-1,2-dihydro-indol-(3Z)-ylidene- methyl]-1H-pyrrole-3-carboxylic acid (2-pyrrolidin-1-ylethyl)-amide |
| 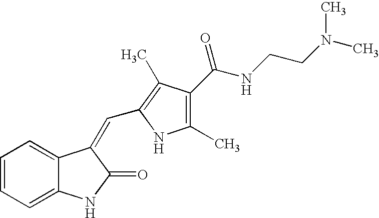 | 2,4-Dimethyl-5-[2-oxo-1,2-dihydro-indol-(3Z)-ylidene- methyl]-2,4-dimethyl-1H-pyrrole-3-car- boxylic acid(2-dimethylaminoethyl)-amide |
| 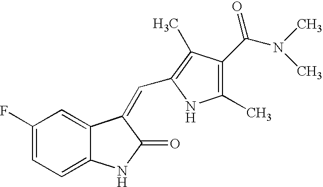 | 5-[5-Fluoro-2-oxo-1,2-dihydro-indol-(3Z)-ylidene- methyl]-2,4-dimethyl-1H-pyrrole-3-car- boxylic acid(2-dimethylaminoethyl)-amide |
| 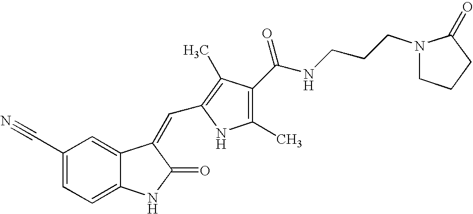 | 5-[5-Cyano-2-oxo-1,2-dihydro-in- dol-(3Z)-ylidenemethyl]-2,4-di- methyl-1H-pyrrole-3-carboxylic acid[3-(2-oxo-pyrrolidin-1-yl)-pro- pyl]-amide |
| 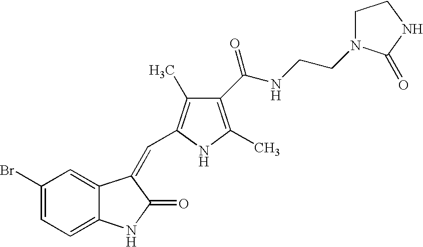 | 5-[5-Bromo-2-oxo-1,2-dihydro-in- dol-(3Z)-ylidenemethyl]-2,4-di- methyl-1H-pyrrole-3-carboxylic acid[2-(2-oxo-imidazolidin-1-yl)-eth- yl]-amide |
| 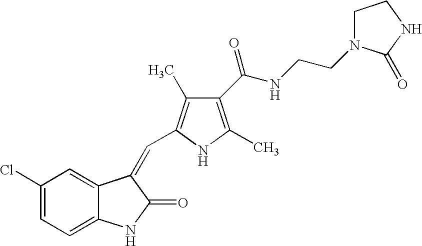 | 5-[5-Chloro-2-oxo-1,2-dihydro-in- dol-(3Z)-ylidenemethyl]-2,4-di- methyl-1H-pyrrole-3-carboxylic acid[2-(2-oxo-imidazolidin-1-yl)-eth- yl]-amide |
| 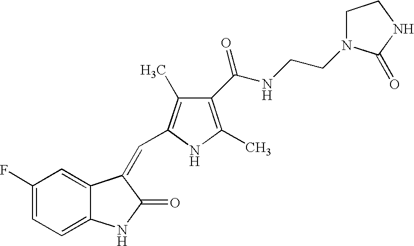 | 5-[5-Fluoro-2-oxo-1,2-dihydro-in- dol-(3Z)-ylidenemethyl]-2,4-di- methyl-1H-pyrrole-3-carboxylic acid[2-(2-oxo-imidazolidin-1-yl)-eth- yl]-amide |
| 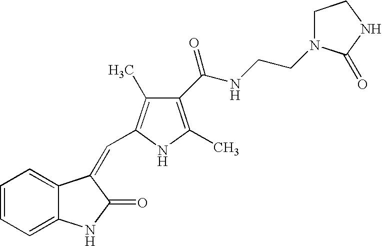 | 2,4-Dimethyl-5-[2-oxo-1,2-di- hydro-indol-(3Z)-ylidene- methyl]-1H-pyrrole-3-car- boxylic acid[2-(2-oxo-imi- dazolidin-1-yl)-ethyl]-amide |
| 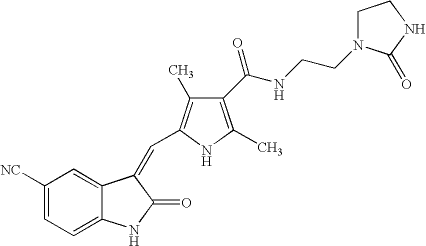 | 5-[5-Cyano-2-oxo-1,2-dihydro-in- dol-(3Z)-ylidenemethyl]-2,4-di- methyl-1H-pyrrole-3-carboxylic acid[2-(2-oxo-imidazolidin-1-yl)-eth- yl]-amide |
| 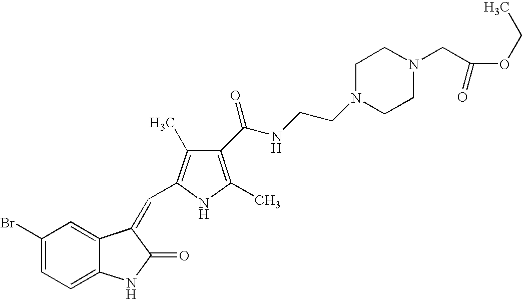 | {4-[2-({5-[5-Bromo-2-oxo-1,2-di- hydro-indol-(3Z)-ylidene- methyl]-2,4-dimethyl-1H-pyr- role-3-carbonyl}-amino)-eth- yl]-piperazin-1-yl}-acetic acid ethyl ester |
| 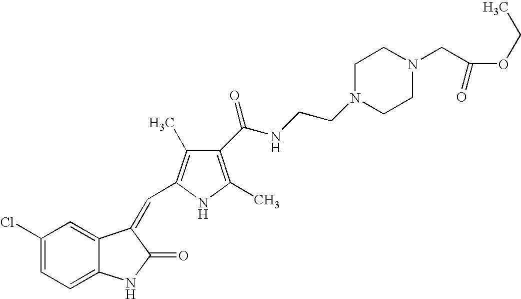 | {4-[2-({5-[5-Chloro-2-oxo-1,2-di- hydro-indol-(3Z)-ylidene- methyl]-2,4-dimethyl-1H-pyr- role-3-carbonyl}-amino)-eth- yl]-piperazin-1-yl}-acetic acid ethyl ester |
| 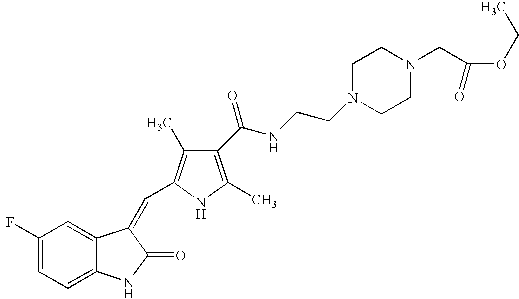 | {4-[2-({5-[5-Fluoro-2-oxo-1,2-di- hydro-indol-(3Z)-ylidene- methyl]-2,4-dimethyl-1H-pyr- role-3-carbonyl}-amino)-eth- yl]-piperazin-1-yl}-acetic acid ethyl ester |
| 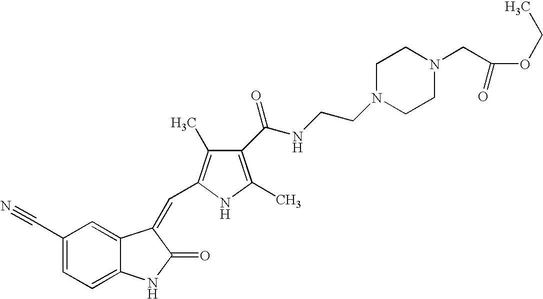 | {4-[2-({5-[5-Cyano-2-oxo-1,2-dihydro-in- dol-(3Z)-ylidenemethyl]-2,4-di- methyl-1H-pyrrole-3-carbonyl}-a- mino)-ethyl]-piperazin-1-yl}-acetic acid ethyl ester |
| 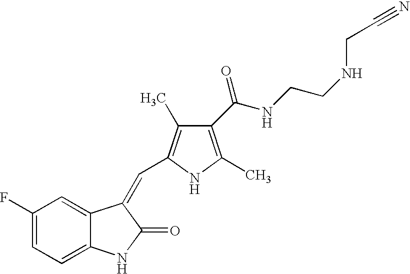 | 5-[5-Fluoro-2-oxo-1,2-dihydro-in- dol-(3Z)-ylidenemethyl]-2,4-dimethyl-1H-pyr- role-3-carboxylic acid[2-(cyano- methyl-amino)-ethyl]-amide |
| 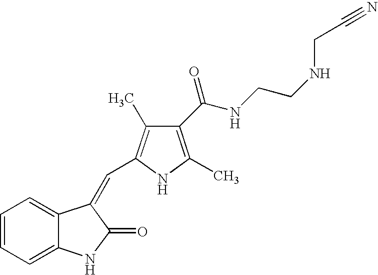 | 2,4-Dimethyl-5-[2-oxo-1,2-dihydro-in- dol-(3Z)-ylidenemethyl]-1H-pyrrole-3-car- boxylic acid[2-(cyanomethyl-amino)-eth- yl]-amide |
| 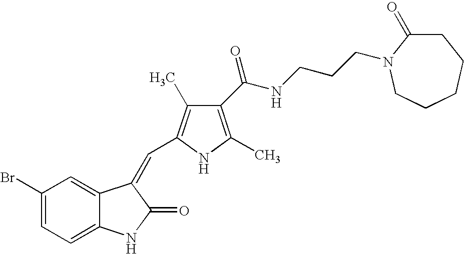 | 5-[5-Bromo-2-oxo-1,2-dihydro-indol-(3Z)-ylidene- methyl]-2,4-dimethyl-1H-pyr- role-3-carboxylic acid[3-(2-oxo-aze- pan-1-yl)-propyl]-amide |
| 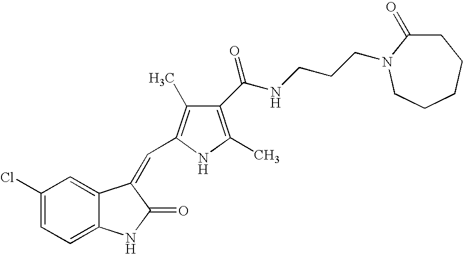 | 5-[5-Chloro-2-oxo-1,2-dihydro-indol-(3Z)-ylidene- methyl]-2,4-dimethyl-1H-pyr- role-3-carboxylic acid[3-(2-oxo-aze- pan-1-yl)-propyl]-amide |
| 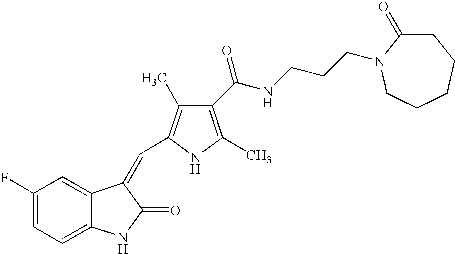 | 5-[5-Fluoro-2-oxo-1,2-dihydro-indol-(3Z)-ylidene- methyl]-2,4-dimethyl-1H-pyr- role-3-carboxylic acid[3-(2-oxo-aze- pan-1-yl)-propyl]-amide |
| 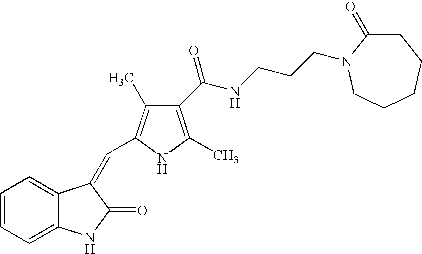 | 2,4-Dimethyl-5-[2-oxo-1,2-dihydro-in- dol-(3Z)-ylidenemethyl]-1H-pyrrole-3-car- boxylic acid[3-(2-oxo-azepan-1-yl)-pro- pyl]-amide |
| 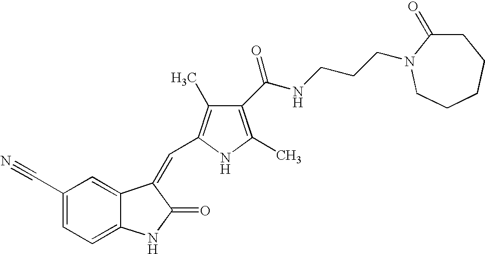 | 5-[5-Cyano-2-oxo-1,2-dihydro-indol-(3Z)-ylidene- methyl]-2,4-dimethyl-1H-pyr- role-3-carboxylic acid[3-(2-oxo-aze- pan-1-yl)-propyl]-amide |
| 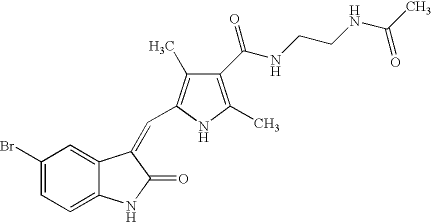 | 5-[5-Bromo-2-oxo-1,2-dihydro-indol-(3Z)-ylidene- methyl]-2,4-dimethyl-1H-pyr- role-3-carboxylic acid(2-acetyl- amino-ethyl)-amide |
| 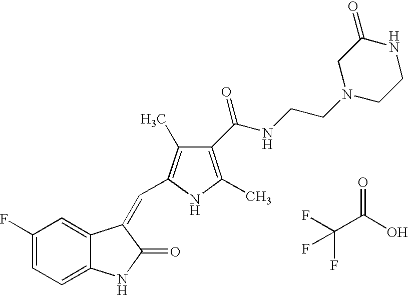 | Trifluoro-acetate4-[2-({5-[5-fluoro-2-oxo-1,2-di- hydro-indol-(3Z)-ylidene- methyl]-2,4-dimethyl-1H-pyr- role-3-carbonyl}-amino)-ethyl]-2-oxo-pipe- razin-1-ium; |
| 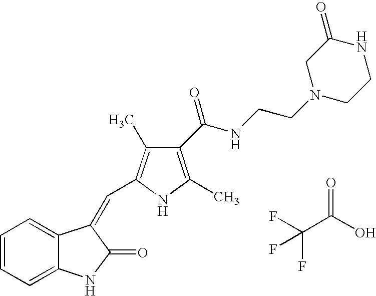 | Trifluoro-acetate4-[2-({2,4-di- methyl-5-[2-oxo-1,2-dihydro-in- dol-(3Z)-ylidenemethyl]-1H-pyr- role-3-carbonyl}-amino)-ethyl]-2-oxo-pipe- razin-1-ium; |
| 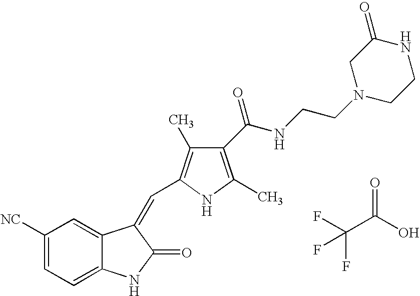 | Trifluoro-acetate4-[2-({5-[5-cyano-2-oxo-1,2-di- hydro-indol-(3Z)-ylidene- methyl]-2,4-dimethyl-1H-pyr- role-3-carbonyl}-amino)-ethyl]-2-oxo-pipe- razin-1-ium; |
| 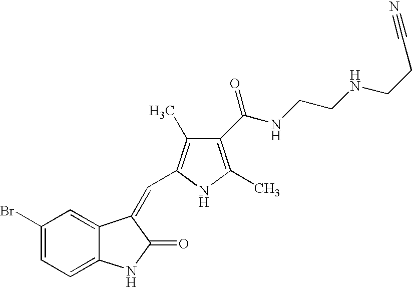 | 5-[5-Bromo-2-oxo-1,2-dihydro-in- dol-(3Z)-ylidenemethyl]-2,4-di- methyl-1H-pyrrole-3-carboxylic acid[2-(2-cyano-ethylamino)-eth- yl]-amide |
| 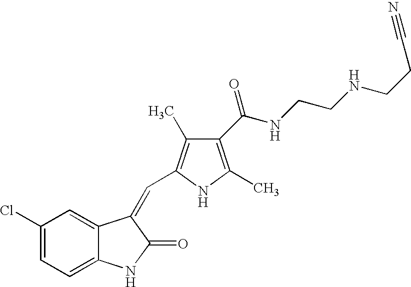 | 5-[5-Chloro-2-oxo-1,2-dihydro-in- dol-(3Z)-ylidenemethyl]-2,4-di- methyl-1H-pyrrole-3-carboxylic acid[2-(2-cyano-ethylamino)-eth- yl]-amide |
| 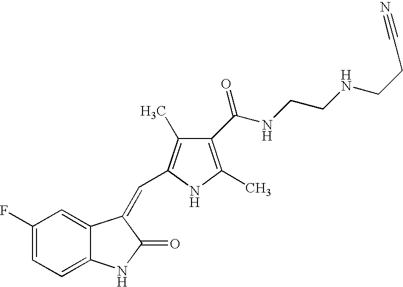 | 5-[5-Fluoro-2-oxo-1,2-dihydro-in- dol-(3Z)-ylidenemethyl]-2,4-di- methyl-1H-pyrrole-3-carboxylic acid[2-(2-cyano-ethylamino)-eth- yl]-amide |
| 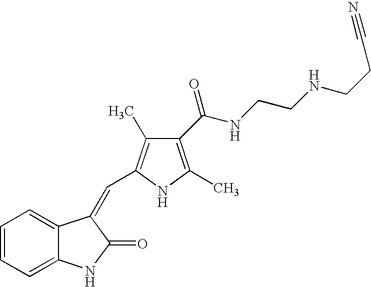 | 2,4-Dimethyl-5-[2-oxo-1,2-dihydro-in- dol-(3Z)-ylidenemethyl]-1H-pyr- role-3-carboxylic acid[2-(2-cya- no-ethylamino)-ethyl]-amide |
| 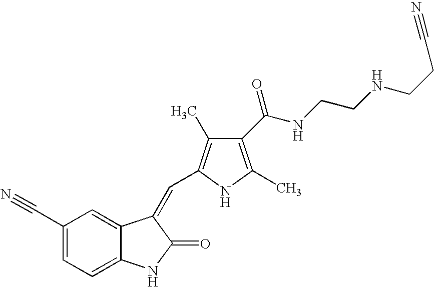 | 5-[5-Cyano-2-oxo-1,2-dihydro-in- dol-(3Z)-ylidenemethyl]-2,4-di- methyl-1H-pyrrole-3-carboxylic acid[2-(2-cyano-ethylamino)-eth- yl]-amide |
| 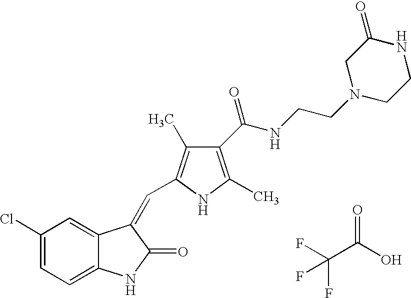 | Trifluoro-acetate4-[2-({5-[5-chloro-2-oxo-1,2-di- hydro-indol-(3Z)-ylidene- methyl]-2,4-dimethyl-1H-pyr- role-3-carbonyl}-amino)-ethyl]-2-oxo-pipe- razin-1-ium; |
| 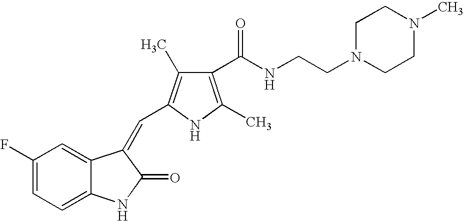 | 5-[5-Fluoro-2-oxo-1,2-dihydro-indol-(3Z)-ylidene- methyl]-2,4-dimethyl-1H-pyr- role-3-carboxylic acid[2-(4-methyl-pipe- razin-1-yl)-ethyl]-amide |
| 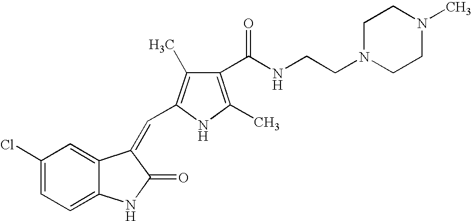 | 5-[5-Chloro-2-oxo-1,2-dihydro-indol-(3Z)-ylidene- methyl]-2,4-dimethyl-1H-pyr- role-3-carboxylic acid[2-(4-methyl-pipe- razin-1-yl)-ethyl]-amide |
| 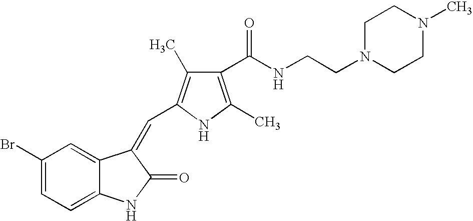 | 5-[5-Bromo-2-oxo-1,2-dihydro-indol-(3Z)-ylidene- methyl]-2,4-dimethyl-1H-pyr- role-3-carboxylic acid[2-(4-methyl-pipe- razin-1-yl)-ethyl]-amide |
| 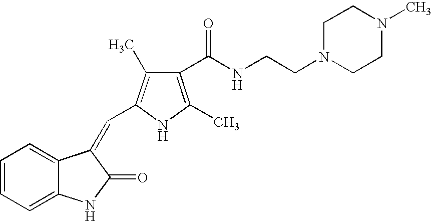 | 2,4-Dimethyl-5-[2-oxo-1,2-dihydro-indol-(3Z)-ylidene- methyl]-1H-pyrrole-3-car- boxylic acid[2-(4-methyl-piperazin-1-yl)-eth- yl]-amide |
| 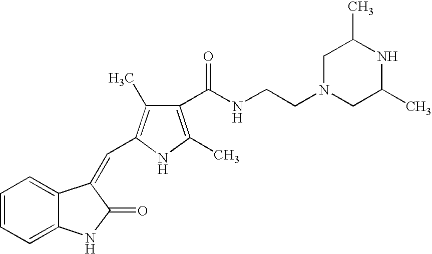 | 2,4-Dimethyl-5-[2-oxo-1,2-dihydro-indol-(3Z)-ylidene- methyl]-1H-pyrrole-3-car- boxylic acid[2-(3,5-dimethyl-pipe- razin-1-yl)-ethyl]-amide |
| 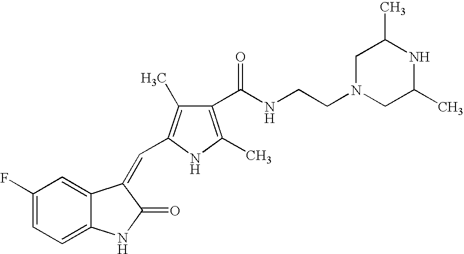 | 5-[5-Fluoro-2-oxo-1,2-dihydro-indol-(3Z)-ylidene- methyl]-2,4-dimethyl-1H-pyr- role-3-carboxylic acid[2-(3,5-di- methyl-piperazin-1-yl)-ethyl]-amide |
| 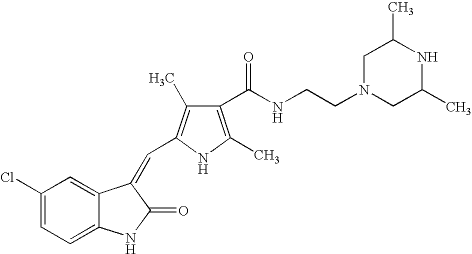 | 5-[5-Chloro-2-oxo-1,2-dihydro-indol-(3Z)-ylidene- methyl]-2,4-dimethyl-1H-pyr- role-3-carboxylic acid[2-(3,5-di- methyl-piperazin-1-yl)-ethyl]-amide |
| 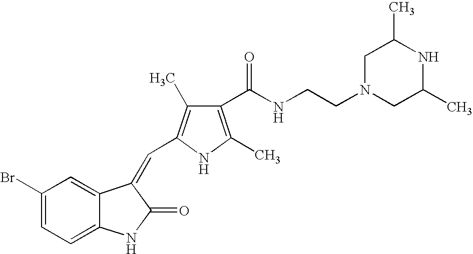 | 5-[5-Bromo-2-oxo-1,2-dihydro-indol-(3Z)-ylidene- methyl]-2,4-dimethyl-1H-pyr- role-3-carboxylic acid[2-(3,5-di- methyl-piperazin-1-yl)-ethyl]-amide |
| 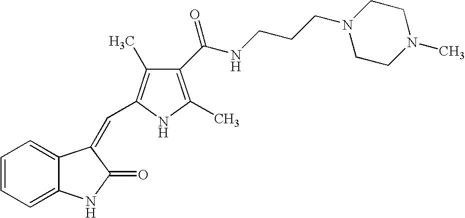 | 2,4-Dimethyl-5-[2-oxo-1,2-di- hydro-indol-(3Z)-ylidene- methyl]-1H-pyrrole-3-carboxylic acid[3-(4-meth- yl-piperazin-1-yl)-pro- pyl]-amide |
| 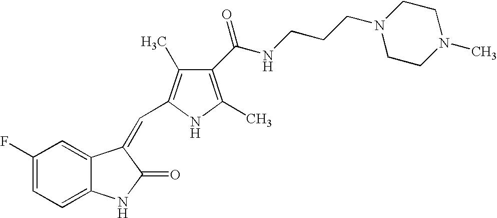 | 5-[5-Fluoro-2-oxo-1,2-di- hydro-indol-(3Z)-ylidene- methyl]-2,4-di- methyl-1H-pyrrole-3-carboxylic acid[3-(4-meth- yl-piperazin-1-yl)-propyl]-amide |
| 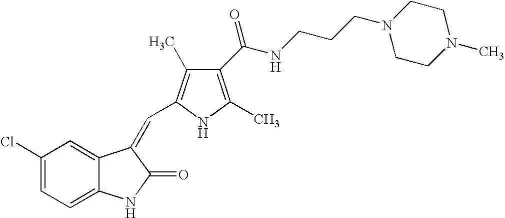 | 5-[5-Chloro-2-oxo-1,2-di- hydro-indol-(3Z)-ylidenemethyl]-2,4-di- methyl-1H-pyrrole-3-carboxylic acid[3-(4-meth- yl-piperazin-1-yl)-propyl]-amide |
| 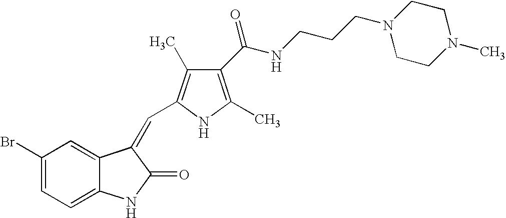 | 5-[5-Bromo-2-oxo-1,2-di- hydro-indol-(3Z)-ylidene- methyl]-2,4-di- methyl-1H-pyrrole-3-carboxylic acid[3-(4-meth- yl-piperazin-1-yl)-propyl]amide |
| 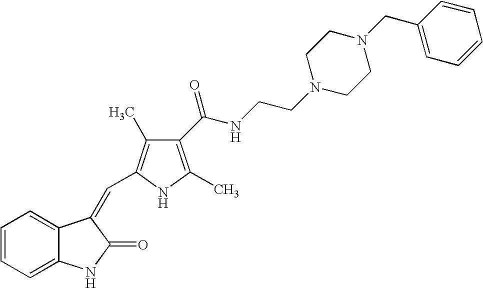 | 2,4-Dimethyl-5-[2-oxo-1,2-di- hydro-indol-(3Z)-ylidene- methyl]-1H-pyrrole-3-car- boxylic acid[2-(4-benzyl-pipe- razin-1-yl)-ethyl]-amide |
| 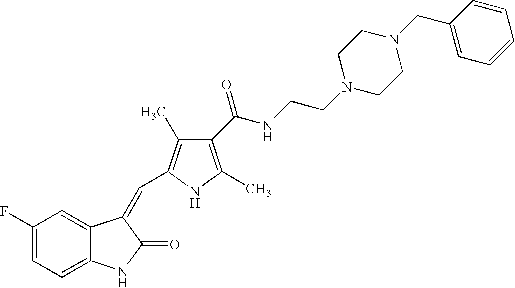 | 5-[5-Fluoro-2-oxo-1,2-di- hydro-indol-(3Z)-ylidene- methyl]-2,4-dimethyl-1H-pyr- role-3-carboxylic acid[2-(4-benzyl-pipe- razin-1-yl)-ethyl]-amide |
| 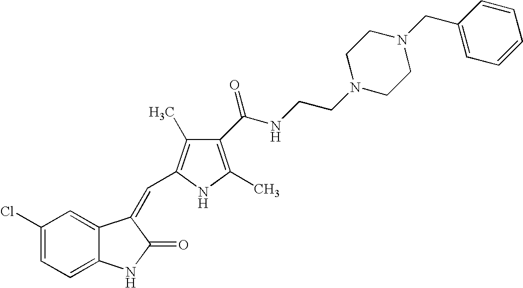 | 5-[5-Chloro-2-oxo-1,2-di- hydro-indol-(3Z)-ylidene- methyl]-2,4-di- methyl-1H-pyrrole-3-car- boxylic acid[2-(4-benzyl-pipe- razin-1-yl)-eth yl]-amide |
| 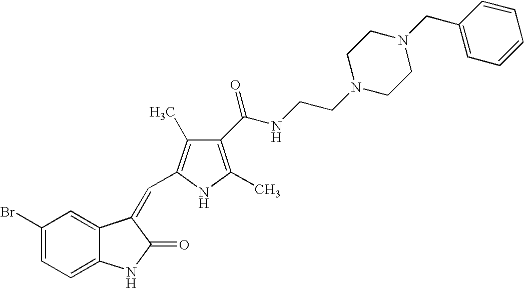 | 5-[5-Bromo-2-oxo-1,2-di- hydro-indol-(3Z)-ylidene- methyl]-2,4-di- methyl-1H-pyrrole-3-carboxylic acid[2-(4-benzyl-piperazin-1-yl)-eth- yl]-amide |
| 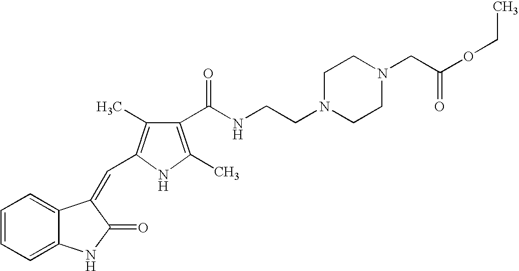 | 5-[5-Chloro-2-oxo-1,2-dihydro-indol-(3Z)-ylidene- methyl]-2,4-dimethyl-1H-pyr- role-3-carboxylic acid(3-pyrrolidin-1yl-2-one)-amide |
| 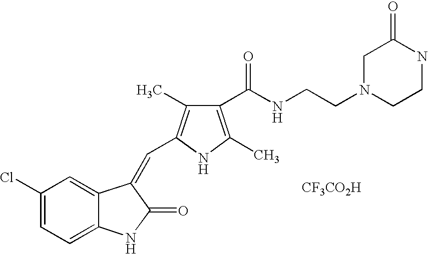 | Trifluoroacetate 4-[2-({5-[5-Chloro-2-oxo-1,2-di- hydro-indol-(3Z)-ylidene- methyl]-2,4-dimethyl-1H-pyrrole-3-car- bonyl}amino)-ethyl]-2-oxo-pipe- razin-1-ium |
| 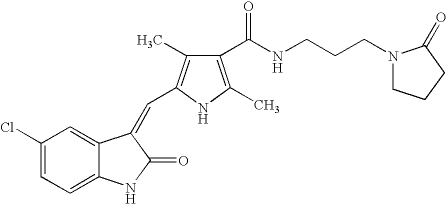 | 5-[5-Chloro-2-oxo-1,2-dihydro-indol-(3Z)-ylidene- methyl]-2,4-dimethyl-1H-pyr- role-3-carboxylic acid(3-pyrrolidin-1-yl-2-one)-amide |
| 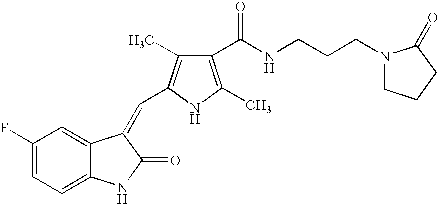 | 5-[5-Fluoro-2-oxo-1,2-dihydro-indol-(3Z)-ylidene- methyl]-2,4-dimethyl-1H-pyr- role-3-carboxylic acid(3-pyrrolidin-1-yl-2-one)-amide |
| 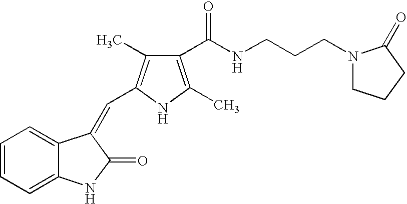 | 5-[2-oxo-1,2-dihydro-indol-(3Z)-ylidene- methyl]-2,4-dimethyl-1H-pyrrole-3-car- boxylic acid(3-pyrrolidin-1-yl-2-one)-amide |
| 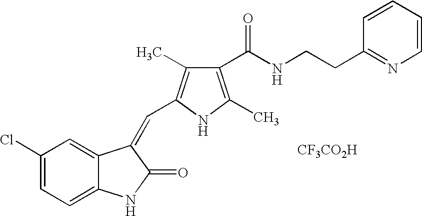 | 5-[5-Chloro-2-oxo-1,2-dihydro-indol-(3Z)-ylidene- methyl]-2,4-dimethyl-1H-pyr- role-3-carboxylic acid(2-pyridin-2-yleth- yl)-amide |
| 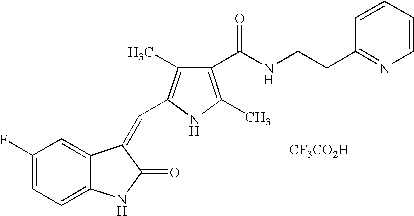 | 5-[5-Fluoro-2-oxo-1,2-dihydro-indol-(3Z)-ylidene- methyl]-2,4-dimethyl-1H-pyr- role-3-carboxylic acid(2-pyridin-2-yleth- yl)-amide trifluoroacetate salt |
| 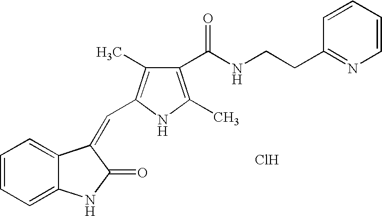 | 5-[2-oxo-1,2-dihydro-indol-(3Z)-ylidene- methyl]-2,4-dimethyl-1H-pyrrole-3-car- boxylic acid(2-pyridin-2-ylethyl)-amide hydrochloride salt |
| 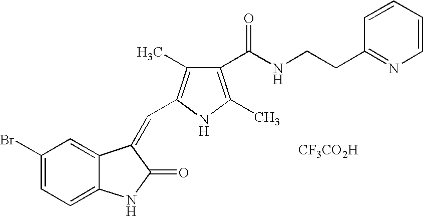 | 5-[5-Bromo-2-oxo-1,2-dihydro-indol-(3Z)-ylidene- methyl]-2,4-dimethyl-1H-pyr- role-3-carboxylic acid(2-pyridin-2-yleth- yl)-amide trifluroracetate salt |
| 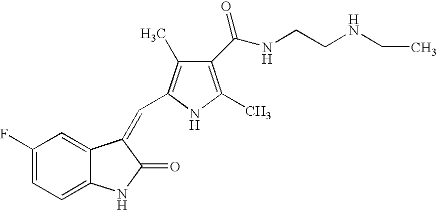 | 5-[5-Fluoro-2-oxo-1,2-dihydro-indol-(3Z)-ylidene- methyl]-2,4-dimethyl-1H-pyr- role-3-carboxylic acid(2-eth- ylaminoethyl)-amide |
| 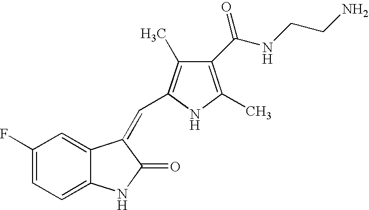 | 5-[5-Fluoro-2-oxo-1,2-dihydro-indol-(3Z)-ylidene- methyl]-2,4-dimethyl-1H-pyr- role-3-carboxylic acid(2-amino- ethyl)-amide |
| 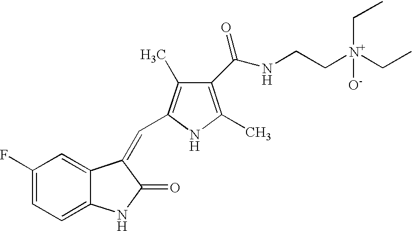 | 5-[5-Fluoro-2-oxo-1,2-dihydro-indol-(3Z)-ylidene- methyl]-2,4-dimethyl-2,4-di- methyl-1H-pyrrole-3-carboxylic acid(2-diethyl-N-oxo- aminoethyl)-amide |
| 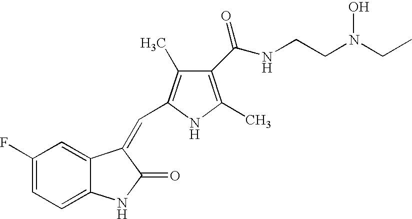 | 5-[5-Fluoro-2-oxo-1,2-dihydro-indol-(3Z)-ylidene- methyl]-2,4-dimethyl-1H-pyr- role-3-carboxylic acid(2-eth- yl-N-hydroxy-aminoethyl)-amide |
| 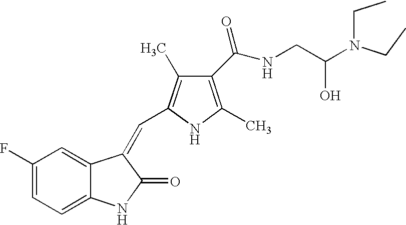 | 5-[5-Fluoro-2-oxo-1,2-dihydro-indol-(3Z)-ylidene- methyl]-1H-pyrrole-3-car- boxylic acid(2-diethylamino-2-hy- droxyethyl)-amide |
| 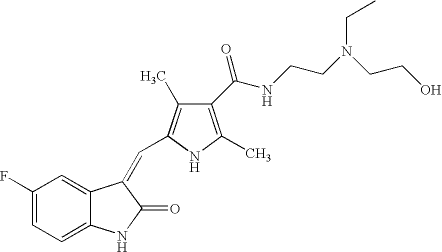 | 5-[5-Fluoro-2-oxo-1,2-dihydro-indol-(3Z)-ylidene- methyl]-2,4-dimethyl-1H-pyr- role-3-carboxylic acid[2-eth- yl-2-(2-hydroxyethyl)aminoethyl]-amide |
| 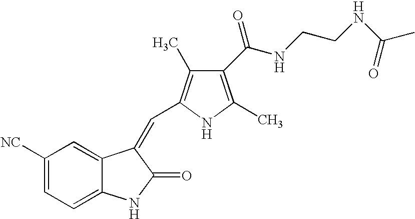 | 5-[5-Cyano-2-oxo-1,2-dihydro-in- dol-(3Z)-ylidenemethyl]-2,4-di- methyl-1H-pyrrole-3-carboxylic acid(2-N-acetylaminoethyl)-amide |
| 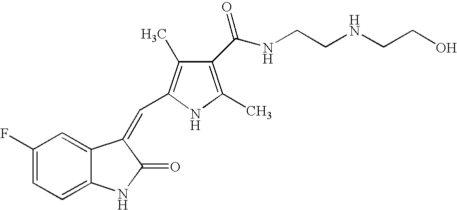 | 5-[5-Fluoro-2-oxo-1,2-dihydro-indol-(3Z)-ylidene- methyl]-1H-pyrrole-3-carboxylic acid[2-(2-hy- droxethylamino)ethyl]-amide |
| 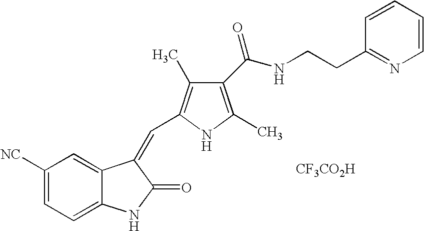 | 5-[5-Cyano-2-oxo-1,2-dihydro-indol-(3Z)-ylidene- methyl]-1H-pyrrole-3-carboxylic acid(2-pyridin-2-ylethyl)-amide trifluoroacetate |
| 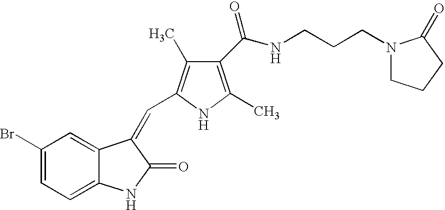 | 5-[5-Bromo-2-oxo-1,2-dihydro-indol-(3Z)-ylidene- methyl]-1H-pyrrole-3-carboxylic acid(3-pyrrolidin-1-yl-2-onepro- pyl)-amide trifluoroacetate |
| 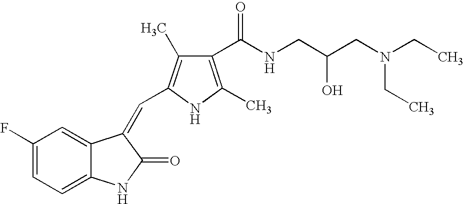 | 5-(5-Fluoro-2-oxo-1,2-dihydro-indol-3-ylidene- methyl)-2,4-dimethyl-1H-pyr- role-3-carboxylic acid(3-di- ethylamino-2-hydroxy-propyl)-amide |
| 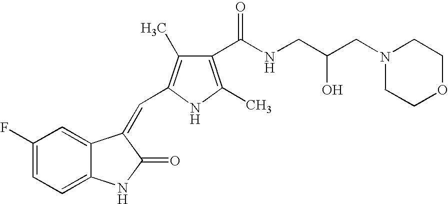 | 5-[5-Fluoro-2-oxo-1,2-dihydro-indol-(3Z)-ylidene- methyl]-2,4-dimethyl-1H-pyr- role-3-carboxylic acid(2-hydroxy-3-morpho- lin-4-yl-propyl)-amide |
| 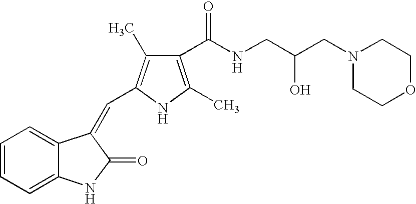 | 2,4-Dimethyl-5-[2-oxo-1,2-dihydro-in- dol-(3Z)-ylidenemethyl]-1H-pyrrole-3-car- boxylic acid(2-hydroxy-3-morpho- lin-4-yl-propyl)-amide |
| 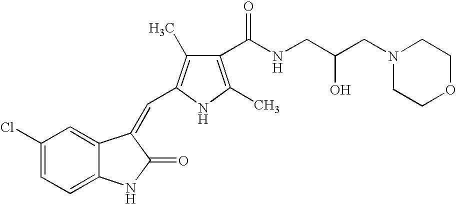 | 5-[5-Chloro-2-oxo-1,2-dihydro-indol-(3Z)-ylidene- methyl]-2,4-dimethyl-1H-pyr- role-3-carboxylic acid(2-hydroxy-3-morpho- lin-4-yl-propyl)-amide |
| 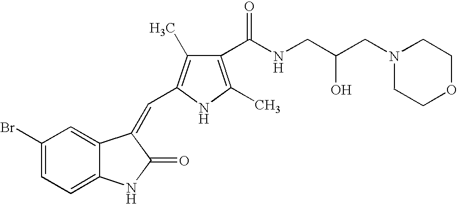 | 5-[5-Bromo-2-oxo-1,2-dihydro-indol-(3Z)-ylidene- methyl]-2,4-dimethyl-1H-pyr- role-3-carboxylic acid(2-hydroxy-3-morpho- lin-4-yl-propyl)-amide |
| 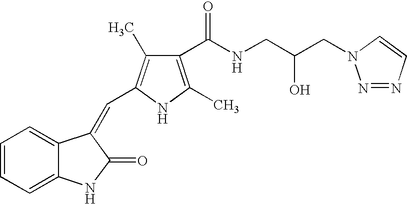 | 2,4-Dimethyl-5-[2-oxo-1,2-dihydro-in- dol-(3Z)-ylidenemethyl]-1H-pyrrole-3-car- boxylic acid(2-hydroxy-3-[1,2,3]tri- azol-1-yl-propyl)-amide |
| 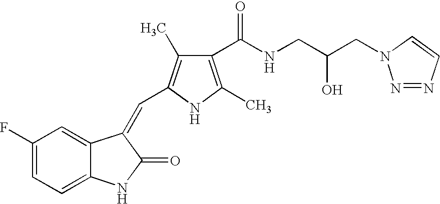 | 5-[5-Fluoro-2-oxo-1,2-dihydro-indol-(3Z)-ylidene- methyl]-2,4-dimethyl-1H-pyr- role-3-carboxylic acid{2-hydroxy-3-[1,2,3]tri- azol-1-yl-propyl)-amide |
| 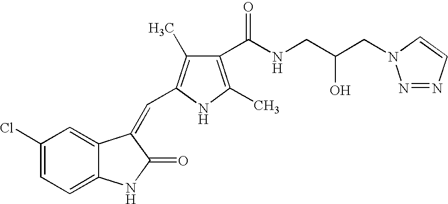 | 5-[5-Chloro-2-oxo-1,2-dihydro-indol-(3Z)-ylidene- methyl]-2,4-dimethyl-1H-pyr- role-3-carboxylic acid(2-hydroxy-3-[1,2,3]tri- azol-1-yl-propyl)-amide |
| 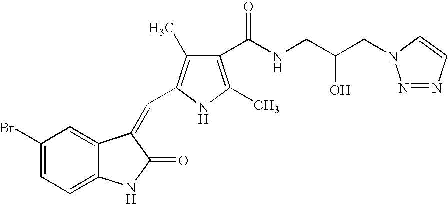 | 5-[5-Bromo-2-oxo-1,2-dihydro-indol-(3Z)-ylidene- methyl]-2,4-dimethyl-1H-pyr- role-3-carboxylic acid(2-hydroxy-3-[1,2,3]tri- azol-1-yl-propyl)-amide |
| 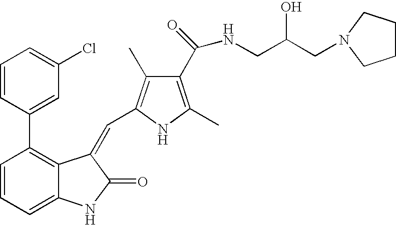 | 5-{(Z)-[4-(3-chlorophenyl)-2-oxo-1,2-di- hydro-3H-indol-3-ylidene]methyl}-N-(2-hy- droxy-3-pyrrolidin-1-ylpropyl)-2,4-di- methyl-1H-pyrrole-3-carboxamide |
| 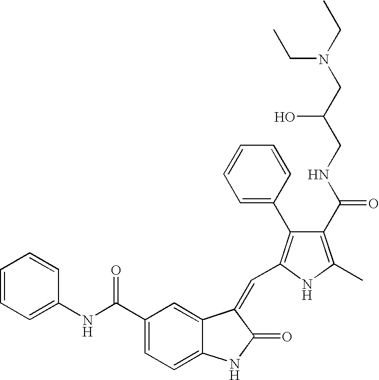 | (3Z)-3-{[4-({[3-(diethylamino)-2-hy- droxypropyl]amino}carbonyl)-5-meth- yl-3-phenyl-1H-pyrrol-2-yl]meth- ylene}-2-oxo-N-phenyl-2,3-di- hydro-1H-indole-5-carboxamide |
| 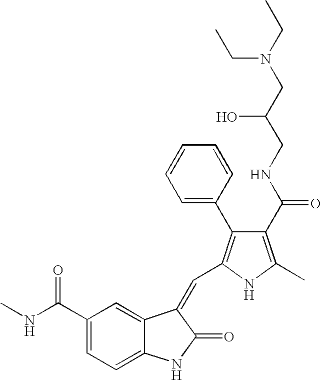 | (3Z)-3-{[4-({[3-(diethylamino)-2-hy- droxypropyl]amino}carbonyl)-5-meth- yl-3-phenyl-1H-pyrrol-2-yl]meth- ylene}-N-methyl-2-oxo-2,3-di- hydro-1H-indole-5-carboxamide |
| 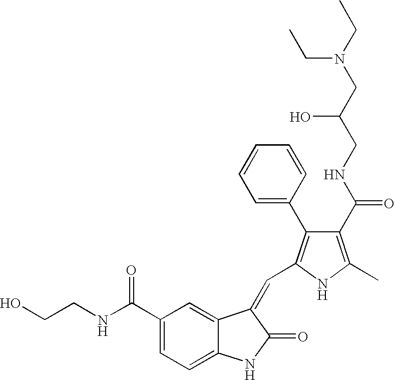 | (3Z)-3-{[4-({[3-(diethylamino)-2-hy- droxypropyl]amino}carbonyl)-5-meth- yl-3-phenyl-1H-pyrrol-2-yl]meth- ylene}-N-(2-hydroxyethyl)-2-oxo-2,3-di- hydro-1H-indole-5-carboxamide |
| 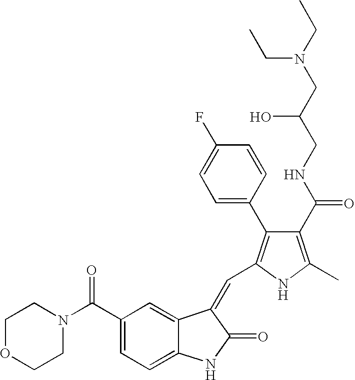 | N-[3-(diethylamino)-2-hydroxypropyl]-4-(4-fluoro- phenyl)-2-methyl-5-{(Z)-[5-(morpho- lin-4-ylcarbonyl)-2-oxo-1,2-di- hydro-3H-indol-3-ylidene]methyl}-1H-pyr- role-3-carboxamide |
| 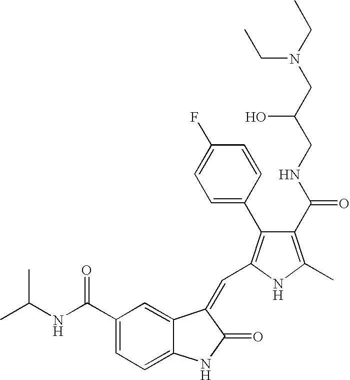 | (3Z)-3-{[4-({[3-(diethylamino)-2-hy- droxpropyl]amino}carbonyl)-3-(4-fluoro- phenyl)-5-methyl-1H-pyrrol-2-yl]meth- ylene}-N-isopropyl-2-oxo-2,3-di- hydro-1H-indole-5-carboxamide |
| 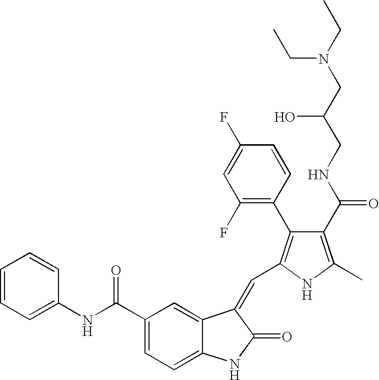 | (3Z)-3-{[4-({[3-(diethylamino)-2-hy- droxypropyl]amino}carbonyl)-3-(2,4-di- fluorophenyl)-5-methyl-1H-pyrrol-2-yl]meth- ylene}-2-oxo-N-phenyl-2,3-di- hydro-1H-indole-5-carboxamide |
| 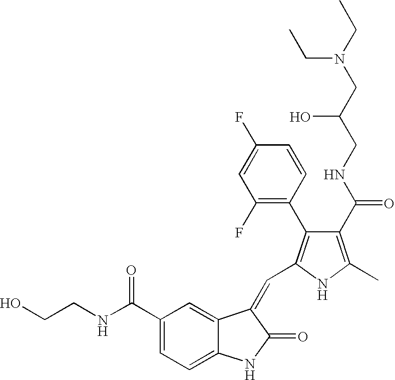 | (3Z)-3-{[4-({[3-(diethylamino)-2-hy- droxypropyl]amino}carbonyl)-3-(2,4-di- fluorophenyl)-5-methyl-1H-pyrrol-2-yl]meth- ylene}-N-(2-hydroxyethyl)-2-oxo-2,3-di- hydro-1H-indole-5-carboxamide |
| 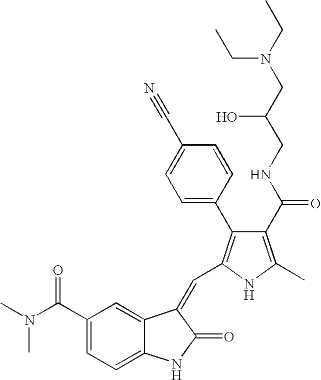 | (3Z)-3-{[3-(4-cyanophenyl)-4-({[3-(di- ethylamino)-2-hy- droxypropyl]amino}carbonyl)-5-meth- yl-1H-pyrrol-2-yl]methylene}-N,N-di- methyl-2-oxo-2,3-dihydro-1H-indole-5-car- boxamide |
| 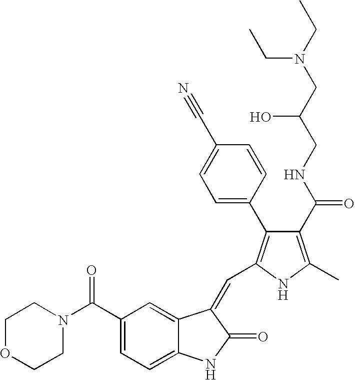 | 4-(4-cyanophenyl)-N-[3-(diethylamino)-2-hy- droxypropyl]-2-methyl-5-{(Z)-[5-(morpho- lin-4-ylcarbonyl)-2-oxo-1,2-di- hydro-3H-indol-3-ylidene]methyl}-1H-pyr- role-3-carboxamide |
| 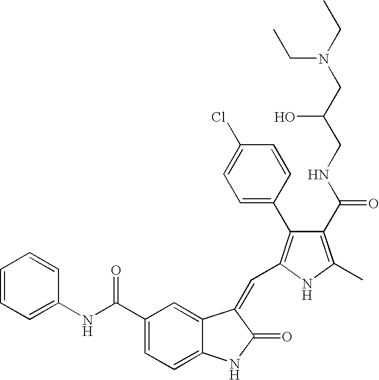 | (3Z)-3-{[3-(4-chlorophenyl)-4-({[3-(di- ethylamino)-2-hy- droxypropyl]amino}carbonyl)-5-meth- yl-1H-pyrrol-2-yl]methylene}-2-oxo-N-phe- nyl-2,3-dihydro-1H-indole-5-car- boxamide |
| 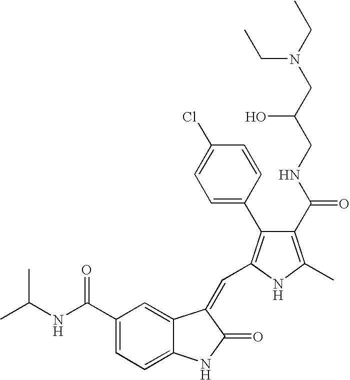 | (3Z)-3-{[3-(4-chlorophenyl)-4-({[3-(di- ethylamino)-2-hy- droxypropyl]amino}carbonyl)-5-meth- yl-1H-pyrrol-2-yl]methylene}-N-iso- propyl-2-oxo-2,3-dihydro-1H-indole-5-car- boxamide |
| 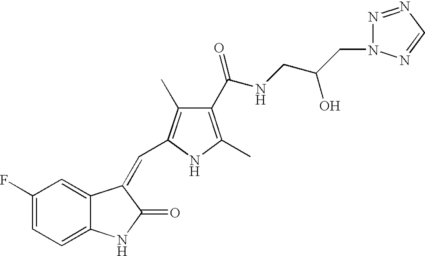 | 5-[(Z)-(5-fluoro-2-oxo-1,2-dihydro-3H-in- dol-3-ylidene)methyl]-N-[2-hydroxy-3-(2H-tetra- azol-2-yl)propyl]-2,4-dimethyl-1H-pyr- role-3-carboxamide |
| 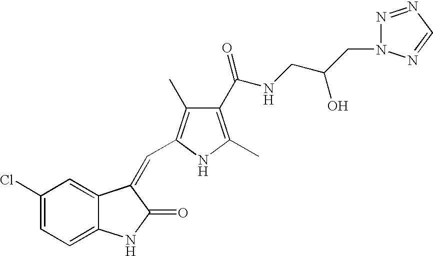 | 5-[(Z)-(5-chloro-2-oxo-1,2-dihydro-3H-in- dol-3-ylidene)methyl]-N-[2-hydroxy-3-(2H-tetra- azol-2-yl)propyl]-2,4-dimethyl-1H-pyr- role-3-carboxamide |
| 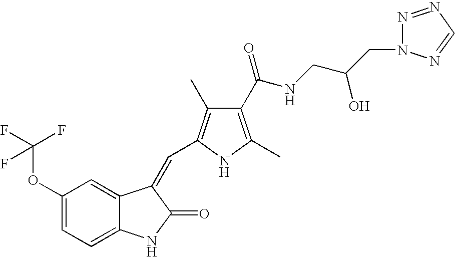 | N-[2-hydroxy-3-(2H-tetraazol-2-yl)pro- pyl]-2,4-dimethyl-5-{(Z)-[2-oxo-5-(tri- fluoromethoxy)-1,2-dihydro-3H-indol-3-yl- idene]methyl}-1H-pyrrole-3-carboxamide |
| 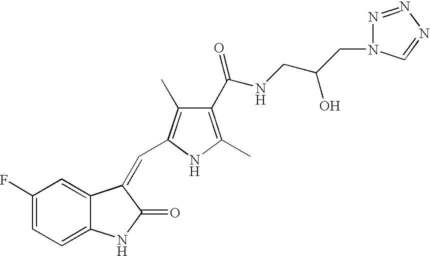 | 5-[(Z)-(5-fluoro-2-oxo-1,2-dihydro-3H-in- dol-3-ylidene)methyl]-N-[2-hydroxy-3-(1H-tetra- azol-1-yl)propyl]-2,4-dimethyl-1H-pyr- role-3-carboxamide |
| 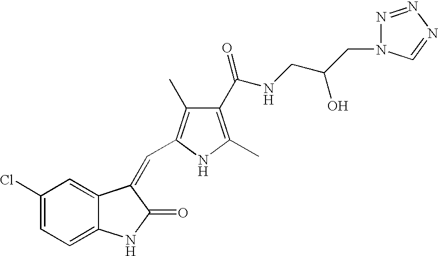 | 5-[(Z)-(5-chloro-2-oxo-1,2-dihydro-3H-in- dol-3-ylidene)methyl]-N-[2-hydroxy-3-(1H-tetra- azol-1-yl)propyl]-2,4-dimethyl-1H-pyr- role-3-carboxamide |
| 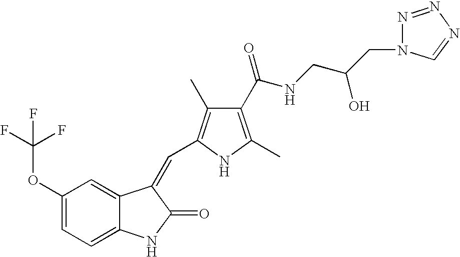 | N-[2-hydroxy-3-(1H-tetraazol-1-yl)pro- pyl]-2,4-dimethyl-5-{(Z)-[2-oxo-5-(tri- fuoromethoxy)-1,2-dihydro-3H-indol-3-yl- idene]methyl}-1H-pyrrole-3-car- boxamide |
| 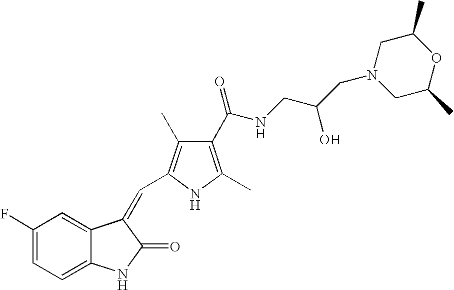 | N-{3-[(2R,6S)-2,6-dimethylmorpholin-4-yl]-2-hy- droxypropyl}-5-[(Z)-(5-fluoro-2-oxo-1,2-di- hydro-3H-indol-3-ylidene)meth- yl]-2,4-dimethyl-1H-pyr- role-3-carboxamide |
| 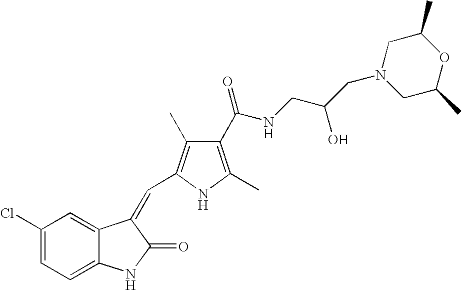 | 5-[(Z)-(5-chloro-2-oxo-1,2-dihydro-3H-in- dol-3-ylidene)methyl]-N-{3-[(2R,6S)-2,6-di- methylmorpholin-4-yl]-2-hy- droxypropyl}-2,4-dimethyl-1H-pyrrole-3-carboxamide |
| 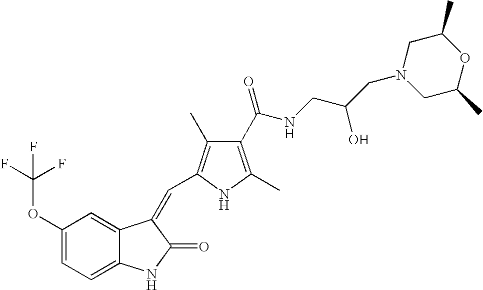 | N-{3-[(2R,6S)-2,6-dimethylmorpholin-4-yl]-2-hy- droxypropyl}-2,4-dimethyl-5-{(Z)-[2-oxo-5-(tri- fluoromethoxy)-1,2-di- hydro-3H-indol-3-ylidene]methyl}-1H-pyr- role-3-carboxamide |
| 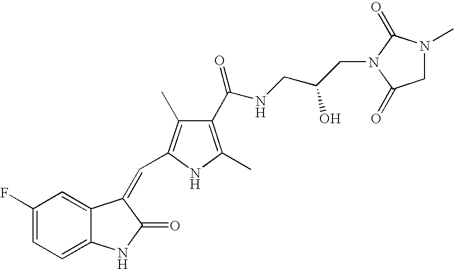 | 5-[(Z)-(5-fluoro-2-oxo-1,2-dihydro-3H-in- dol-3-ylidene)methyl]-N-[(2R)-2-hy- droxy-3-(3-methyl-2,5-di- oxoimidazolidin-1-yl)propyl]-2,4-di- methyl-1H-pyrrole-3-carboxamide |
| 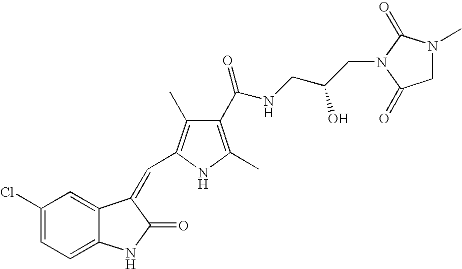 | 5-[(Z)-(5-chloro-2-oxo-1,2-dihydro-3H-in- dol-3-ylidene)methyl]-N-[(2R)-2-hy- droxy-3-(3-methyl-2,5-di- oxoimidazolidin-1-yl)propyl]-2,4-di- methyl-1H-pyrrole-3-carboxamide |
| 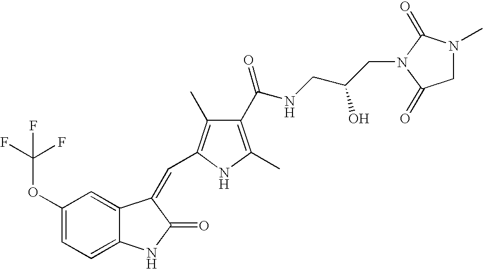 | N-[(2R)-2-hydroxy-3-(3-methyl-2,5-di- oxoimidazolidin-1-yl)propyl]-2,4-di- methyl-5-{(Z)-[2-oxo-5-(tri- fluoromethoxy)-1,2-dihydro-3H-indol-3-yl- idene]methyl}-1H-pyrrole-3-car- boxamide |
| 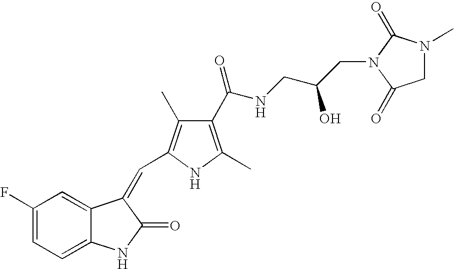 | 5-[(Z)-(5-fluoro-2-oxo-1,2-dihydro-3H-in- dol-3-ylidene)methyl]-N-[(2S)-2-hy- droxy-3-(3-methyl-2,5-di- oxoimidazolidin-1-yl)propyl]-2,4-di- methyl-1H-pyrrole-3-carboxamide |
| 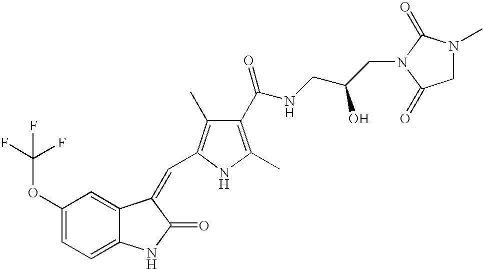 | N-[(2S)-2-hydroxy-3-(3-methyl-2,5-di- oxoimidazolidin-1-yl)propyl]-2,4-di- methyl-5-{(Z)-[2-oxo-5-(tri- fluoromethoxy)-1,2-dihydro-3H-indol-3-yl- idene]methyl}-1H-pyrrole-3-carboxamide |
| 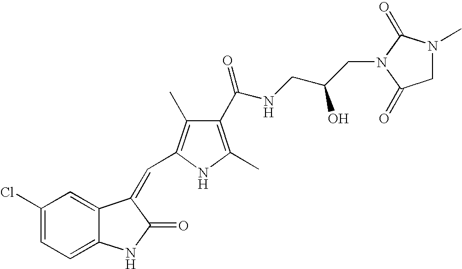 | 5-[(Z)-(5-chloro-2-oxo-1,2-dihydro-3H-in- dol-3-ylidene)methyl]-N-[(2S)-2-hy- droxy-3-(3-methyl-2,5-di- oxoimidazolidin-1-yl)propyl]-2,4-di- methyl-1H-pyrrole-3-carboxamide |
| 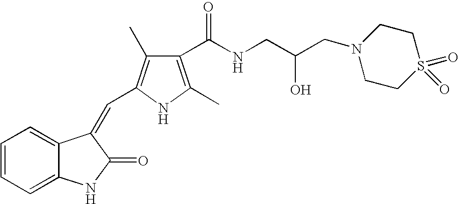 | N-[3-(1,1-dioxidothiomorpholin-4-yl)-2-hy- droxypropyl]-2,4-dimethyl-5-[{Z)-(2-oxo-1,2-di- hydro-3H-indol-3-yl- idene)methyl]-1H-pyrrole-3-carboxamide |
| 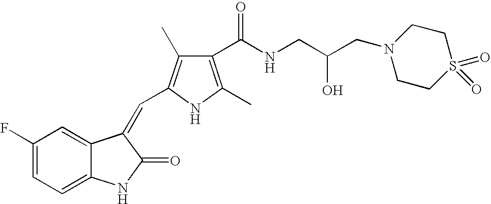 | N-[3-(1,1-dioxidothiomorpholin-4-yl)-2-hy- droxypropyl]-5-[(Z)-(5-fluoro-2-oxo-1,2-di- hydro-3H-indol-3-ylidene)methyl]-2,4-di- methyl-1H-pyrrole-3-carboxamide |
| 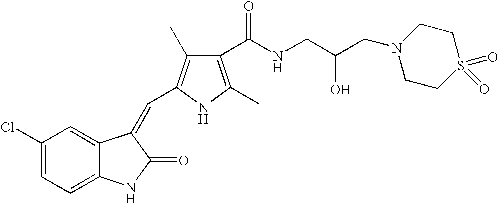 | 5-[(Z)-(5-chloro-2-oxo-1,2-dihydro-3H-in- dol-3-ylidene)methyl]-N-[3-(1,1-di- oxidothiomorpholin-4-yl)-2-hy- droxypropyl]-2,4-dimethyl-1H-pyrrole-3-car- boxamide |
| 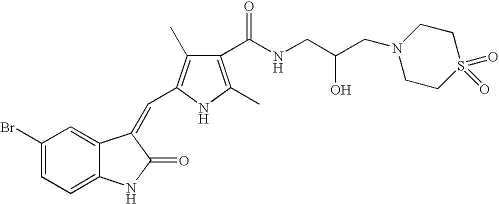 | 5-[(Z)-(5-bromo-2-oxo-1,2-dihydro-3H-in- dol-3-ylidene)methyl]-N-[3-(1,1-di- oxidothiomorpholin-4-yl)-2-hy- droxypropyl]-2,4-dimethyl-1H-pyrrole-3-car- boxamide |
| 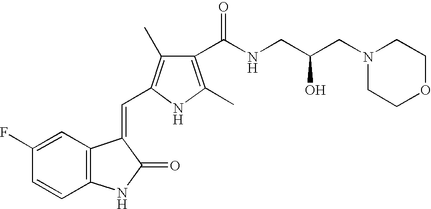 | 5-[(Z)-(5-fluoro-2-oxo-1,2-dihydro-3H-in- dol-3-ylidene)methyl]-N-[(2S)-2-hy- droxy-3-morpholin-4-ylpropyl]-2,4-di- methyl-1H-pyrrole-3-carboxamide |
| 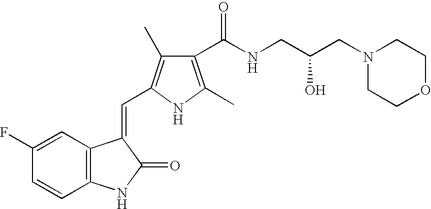 | 5-[(Z)-(5-fluoro-2-oxo-1,2-dihydro-3H-in- dol-3-ylidene)methyl]-N-[(2R)-2-hy- droxy-3-morpholin-4-ylpropyl]-2,4-di- methyl-1H-pyrrole-3-carboxamide |
| 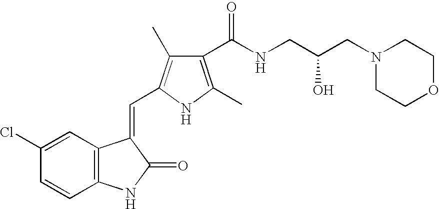 | 5-[(Z)-(5-chloro-2-oxo-1,2-dihydro-3H-in- dol-3-ylidene)methyl]-N-[(2R)-2-hy- droxy-3-morpholin-4-ylpropyl]-2,4-di- methyl-1H-pyrrole-3-carboxamide |
| 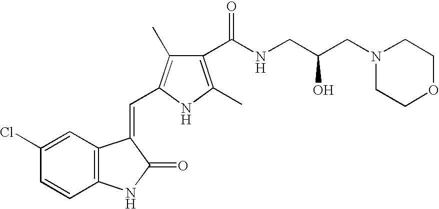 | 5-[(Z)-(5-chloro-2-oxo-1,2-dihydro-3H-in- dol-3-ylidene)methyl]-N-[(2S)-2-hy- droxy-3-morpholin-4-ylpropyl]-2,4-di- methyl-1H-pyrrole-3-carboxamide |
| 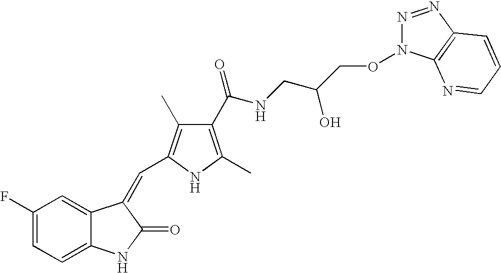 | 5-(5-(Z)-fluoro-2-oxo-1,2-dihydro-indol-3-ylidene- methyl)-2,4-dimethyl-1H-pyrrole-3-carboxylic acid[2-hydroxy-3-([1,2,3]tri- azolo[4,5-b]pyridin-3-yl- oxy)-propyl]-amide |
| 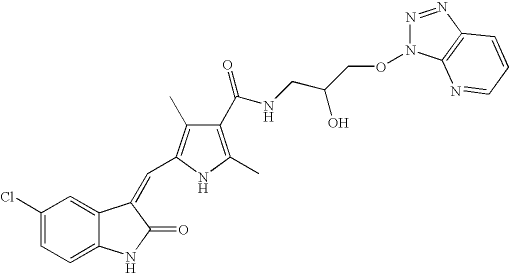 | 5-(5-(Z)-chloro-2-oxo-1,2-dihydro-indol-3-ylidene- methyl)-2,4-dimethyl-1H-pyr- role-3-carboxylic acid[2-hy- droxy-3-([1,2,3]triazolo[4,5-b]pyridin-3-yl- oxy)-propyl]-amide |

As used herein, the term “pharmaceutically acceptable salt” refers to those salts which retain the biological effectiveness and properties of the parent compound. Such salts include:

(i) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid such as the L-malate salt of 5-(5-fluoro-2-oxo-1,2-dihydroindol-3-ylidenemethyl)-2,4-dimethyl-1H-pyrrole-3-carboxylic acid(2-diethylaminoethyl)amide; or

(ii) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucarnine, and the like.

As utilized herein, the term “prodrug” includes any compounds that, when administered to a biological system, are converted into the active indolinone contemplated for use in the invention either as a result of spontaneous chemical reaction(s), enzyme catalyzed reaction(s), metabolic reaction(s), or the like. A “prodrug” also refers to an agent which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water solubility is beneficial. A further example of a prodrug might be a short polypeptide bonded to a carboxy group wherein metabolic removal of the. polypeptide group releases the active compound.

Formulation

In one aspect of the invention formulation, a therapeutically effective amount of an indolinone is utilized in the invention formulation.

As used herein, a “pharmaceutically acceptable carrier” refers to a carrier or diluent that does not abrogate the biological activity and/or properties of the administered compound while facilitating administration by, for example, stabilizing or solubilizing the compound. Suitable pharmaceutically acceptable carriers include, without limitation, one or more pharmaceutically acceptable diluents, one or more pharmaceutically acceptable binders, one or more pharmaceutically acceptable disintegrants, one or more pharmaceutically acceptable lubricants.

The term “pharmaceutically acceptable” or “pharmaceutical” as used herein refers to solutions or components of the formulation that do not prevent the therapeutic compound from exerting a therapeutic effect and do not cause unacceptable adverse side effects. Examples of pharmaceutically acceptable reagents are provided in The United States Pharmacopeia The National Formulary, United States Pharnacopeial Convention, Inc., Rockville, Md. 1990 and FDA Inactive Ingredient Guide 1990, 1996 issued by the Division of Drug Information Resources (both are hereby incorporated by reference herein, including any drawings). Unacceptable side effects vary for different diseases. Generally, the more severe the disease the more toxic effects which will be tolerated. Unacceptable side effects for different diseases are known in the art.

Although all permutations of specific components within the invention formulations are contemplated to be within the scope of the present invention, the following combinations of one or more specific pharmaceutically acceptable carrier(s) and specific indolinones are preferred in one aspect of the invention.

One aspect of the invention is a formulation suitable for oral administration, the formulation is solid and the pharmaceutically acceptable carrier comprises one or more pharmaceutically acceptable diluents, one or more pharmaceutically acceptable binders, one or more pharmaceutically acceptable disintegrants and one or more pharmaceutically acceptable lubricants.

In one aspect of the invention formulation, the formulation is suitable for oral or parenteral administration. A preferred embodiment of this aspect has an indolinone of Formula I, and pharmaceutically active salts, prodrugs, derivatives, and analogs thereof.

Suitable pharmaceutically acceptable diluents include without limitation pregelatinized starch, lactose, monohydrate or lactose anhydrous, mannitol, microcrystalline cellulose, and the like, and suitable combinations of two or more thereof.

Suitable pharmaceutically acceptable binders include without limitation polyvinylpyrrolidone (povidone), hydroxylpropyl cellulose, carboxymethyl cellulose (CMC), hydroxypropylmethylcellulose (HPMC), starch, and the like, and suitable combinations of two or more thereof.

Suitable pharmaceutically acceptable disintegrants include without limitation sodium starch glycollate, crosscarnellose, crospovidone, sodium carboxymethylcellulose, calcium carboxymethylcellulose, starch and the like, and suitable combinations of two or more thereof.

Suitable pharmaceutically acceptable lubricants include without limitation magnesium stearate, stearic acid, sodium stearyl fumarate, PEG (3,000-10,000), glyceryl behenate and the like, and suitable combinations of any two or more thereof.

In another aspect, the carrier further includes pharmaceutically acceptable flow enhancers. These include without limitation colloidal silicon dioxide, talc, and the like, and suitable combinations of any two or more thereof.

In another aspect of the invention, the formulation further includes permeability and penetrating enhancers. These include ionic compounds (e.g., 3,5-diidosalicylate sodium) dimethylsulfoxide and related compounds (e.g., decylmethyl sulfoxide) azone, and related compounds (e.g., N-alkyl-dihydro-1,4-oxazepine-5,7-diones), solvents, and related compounds (e.g., ethanol, dimethyl acetamide, dimethylformamide) fatty alcohols, fatty acids and enzymes (e.g., acid phosphatase and papin). These are other examples of permeability and pentration enhancers can be found in Pharmaceutical Skin Penetration Enhancement, K. A. Walters and J. Hadgraft, Eds. (Dekker, N.Y., 1993).

In another aspect of the invention formulation suitable for oral administration, the indolinone is solubilized by combining it with a molar equivalent of an acid solution. A preferred embodiment of this aspect has the indolinone of Formula I, and pharmaceutically active salts, prodrugs, derivatives, and analogs thereof.

The term “solubilized” as used herein refers to dissolving of a substance in a fluid and/or adsorption of fluid molecules on the surface of the substance to assist in such dissolving. In one aspect, “solubilized” refers to hydration of a substance in water.

The term “molar equivalent” as used herein refers to equal or similar molar amounts of a test substance as compared to a reference substance. a

The term “acid solution” as used herein refers to an acidic solution, typically one which has a pH lower than 7 and is capable of reacting with a basic solution. Preferably the acid in the acid solution is selected from the group consisting of hydrochloric acid, sulfuric acid, formic acid, lactic acid, malic acid, maleic acid, succinic acid, acetic acid, methane sulfonic acid, benzene sulfonic acid, phosphoric acid, malonic acid and the like,.and suitable combinations of two or more hereof.

In another aspect of the invention formulation suitable for oral administration, the pharmaceutically acceptable carrier further comprises one or more buffers. A preferred embodiment of this aspect has the indolinone of Formula I, and pharmaceutically active salts, prodrugs, derivatives, and analogs thereof.

The term “buffer” as used herein refers to a substance, preferably in a solution, that resists a change of quality. Preferably a buffer is a solution that resists a change to a pH, such as a substance in a solution capable of neutralizing both acids and bases and therefore maintaining an original acidity or basicity of a solution. Suitable buffers include acetate, citrate, phosphate buffer, ascorbate, hydrochloric acid buffer, Tris-HCl buffer, sodium phosphate, sodium carbonate, sodium hydroxide, glutamate, glycine, Tris base buffers, and the like, and suitable combinations of two or more hereof. Most preferably, the buffer is sodium phosphate buffer.

In one embodiment, the buffer pH is three pH units lower than the pK_{b} of the ionizable substituted indolinone. Preferably, the buffer has a molarity (i.e., molar concentration, measured in moles per liter (M)) between 0.01 M and 0.1 M.

The term “pK_{b}” as used herein refers to the negative logarithm of the basicity constant, the basicity constant being the product of the concentration of the hydroxyl ion and the concentration of the conjugated acid, divided by the concentration of the base (the basicity constant is also sometimes referred to as the equilibrium constant).

Because the formulations have been shown to have a therapeutic effect with the components described herein, formulations of the present invention may also “consist essentially of” or “consist of” these components.

In another aspect of the invention formulation suitable for oral administration, the pharmaceutically acceptable carrier does not contain any pharmaceutically acceptable surfactants. In other words, the formulation is prepared in the absence of a surfactant or surfactants.

The term “pharmaceutically acceptable surfactant” as used herein with respect to formulations refers to a compound that can solubilize hydrophobic compounds into aqueous solutions. Suitable surfactants include non-ionic surfactants, anionic surfactants, and the like, and suitable combinations of two or more thereof. Examples of pharmaceutically acceptable non-ionic surfactants include but are not limited to polyoxyethylene sorbitan fatty acid esters (e.g., POLYSORBATE 80®, and the like), glyceryl monooleate, sorbitan monooleate, lecithin, polyvinyl alcohol, ethylene oxide copolymers (such as PLURONIC™ (a polyether), TETRONIC™ (BASF), and the like), polyol moieties, sorbitan esters, and the like, and suitable combinations of two or more hereof. Preferably ethoxylated castor oils, such as CREMOPHOR EL®, are used for the formulation of hydrophobic pharmaceutical agents, such as the ionizable substituted indolinones contemplated for use in the present invention. The term “ethoxylated castor oil” as used herein refers to castor oil that is modified with at least one oxygen containing moiety. In particular the term refers to castor oil comprising at least one ethoxyl moiety.

Further, the term “pharmaceutically acceptable surfactant” includes pharmaceutically acceptable non-ionic surfactants such as copolymers of ethylene glycol nd propylene glycol (for example, polyoxyethylenepolypropylene glycols (such as POLOXAMER® 68 (BASF Corp.)) or a mono fatty acid ester of polyoxyethylene (20) sorbitan monooleate (TWEEN® 80), polyoxyethylene (20) sorbitan monostearate (TWEEN(® 60), polyoxyethylene (20) sorbitan monopalmitate (TWEEN® 40), polyoxyethylene (20) sorbitan monolaurate (TWEEN® 20), and the like); polyoxyethylene castor oil derivatives (such as polyoxyethyleneglycerol-triricinoleate, polyoxyl 35 castor oil (CREMOPHOR® EL, BASF Corp.), and the like); polyoxyethyleneglycerol oxystearate (such as CREMOPHOR® RH 40 (polyethyleneglycol 40 hydrogenated castor oil), CREMOPHOR® RH 60 (polyethyleneglycol 60 hydrogenated castor oil), BASF Corp.), and the like); and the like); pharmaceutically acceptable anionic surfactants, e.g., sodiumlauryl sulfate (SLS); and the like; and suitable combinations of two or more hereof.

In a further aspect of the invention formulation suitable for oral administration, the pharmaceutically acceptable carrier further comprises one or more pharmaceutically acceptable preservatives. A preferred embodiment of this aspect has the indolinone of Formula I, and pharmaceutically active salts, prodrugs, derivatives, and analogs thereof.

Preferably, each of the one or more pharmaceutically acceptable preservatives is selected from the group consisting of benzyl alcohol, methyl paraben, ethyl paraben, propyl paraban, phenol, and the like, and suitable combinations of two or more hereof.

In yet another aspect of the invention formulation suitable for oral administration, the pharmaceutically acceptable carrier further comprises one or more antioxidants. A preferred embodiment of this aspect has the indolinone of Formula I, and pharmaceutically active salts, prodrugs, derivatives, and analogs thereof.

The term “antioxidant” as used herein refers to a substance that inhibits oxidation or reactions promoted by, for example, oxygen or peroxides. Suitable antioxidants include sodium meta-bisulfite, EDTA, sodium ascorbate, ascorbic acid, ascorbic acid palmitate, benzyl alcohol, alpha-tocopherol and the like, and suitable combinations of two or more hereof. Preferably the antioxidant is alpha-tocopherol.

In one aspect of the invention formulation suitable for oral administration, the pharmaceutically acceptable carrier comprises one or more polyoxyhydrocarbyl compounds. A preferred embodiment of this aspect has the indolinone of Formula I, and pharmaceutically active salts, prodrugs, derivatives, and analogs thereof.

In preferred embodiments of this aspect of the invention formulation suitable for oral administration, the one or more polyoxyhydrocarbyl compounds are independently selected from the group consisting of: water soluble carbohydrates, water soluble carbohydrate derivatives, polypeptides, water soluble polymers, water soluble mixed oxyalkylene polymers, and the polymeric form of ethylene glycol. Preferably, the one or more polyoxyhydrocarbyl compounds are poly(ethylene glycol) (PEG) or PEG derivatives. More preferably, PEG may vary in molecular weight from about 1000 daltons to about 20,000 daltons.

In another embodiment the composition further comprises one or more polyoxyhydrocarbyl compounds selected from the group consisting of polyethylene glycol 3350, polyethylene glycol 1000, and the like, and suitable combinations of two or more hereof, although polyoxyhydrocarbyl compounds listed previously can also be used in some cases. Preferably, the one or more polyoxyhydrocarbyl compounds is polyethylene glycol of greater than 1000 daltons.

In another aspect of the invention formulation suitable for oral administration, the pharmaceutically acceptable carrier comprises one or more polyglycolized lipids. A preferred embodiment of this aspect has the indolinone of Formula I, and pharmaceutically active salts, prodrugs, derivatives, and analogs thereof.

The term “polyglycolized lipids” as used herein refers to mixtures of mono glycerides, diglycerides, or triglycerides and polyethyleneglycol monoesters and diesters formed by the partial alcoholysis of vegetable oil using PEG of 200 g/mol to 2,000 g/mol or by the esterification of fatty acids using PEG 200 g/mol to 2,000 g/mol and glycerols. Preferably these include GELUCIRE® 35/10, GELUCIRE® 44/14, GELUCIRE® 46/07, GELUCIRE® 50/13, GELUCIRE® 53/10, and LABRASOL®.

In an additional aspect of the invention formulation suitable for oral administration, the pharmaceutically acceptable carrier comprises one or more pharmaceutically acceptable granulating agents. A preferred embodiment of this aspect has the indolinone Formula I, and pharmaceutically active salts, prodrugs, derivatives, and analogs thereof. Suitable granulating agents include without limitation, microcrystalline cellulose, starch, calcium carbonate, pectin, crospovidone, polyplasdone, and the like, and suitable combinations of two or more thereof.

In an additional aspect, the invention provides pharmaceutically acceptable compositions containing an indolinone. Preferred pharmaceutically acceptable compositions of the present invention are selected from the group comprising the invention formulation suitable for oral administration, a hard gelatin capsule filled with the invention formulation suitable for oral administration, the invention formulation suitable for oral administration admixed with a granulating agent to form a dry solid composition processed into a capsule or pressed to form a tablet. In preferred embodiments, the invention formulation suitable for oral administration is encapsulated in a hard gelatin capsule.

The capsule sizes that may be used in the practice of the preferred embodiments of the present invention are capsules that range from size 00 to size 4.

In an additional aspect, the invention features a method of preparing a formulation for oral administration comprising adding to a salt solution, formed in situ by admixing a molar equivalent of an acid solution with an ionizable substituted indolinone and/or one or more buffers. In a preferred embodiment, the one or more buffers are added to the salt solution.

In a preferred embodiment of the method of preparing a formulation, the acid solution is selected from the group consisting of hydrochloric acid, sulfuric acid, formic acid, lactic acid, malic acid, maleic acid, malonic acid and the like, and suitable combinations of two or more hereof.

In a further preferred embodiment of the method of preparing a formulation, the method also includes sterilizing the formulation. Preferably, the sterilizing is done by gamma irradiation, autoclaving or aseptic processing.

In preferred embodiments, the formulations of the invention comprise a malate salt of an indolinone compound, preferably the L-malate salt of an indolinone compound, and more preferably the L-malate salt of 5-(5-fluoro-2-oxo-1,2-dihydroindol-3-ylidenemethyl)-2,4-dimethyl-1H-pyrrole-3-carboxylic acid(2-diethylaminoethyl)amide.

In preferred embodiments, the formulation of the invention has a bulk density of at least about 0.5 kg/L, preferably at least about 0.55, 0.56, 0.57, 0.58, 0.59. 0.60, 0.61, 0.62, 0.63, 0.64, 0.65, 0.66, 0.67, 0.69 or 0.7 kg/L.

In other preferred embodiments, the bulk density of the solid formulation comprising an indolinone of formula I is from about 0.6 kg/L to about 0.7 kg/L or from about 0.5 kg/L to about 0.7 kg/L.

In still other preferred embodiments, the bulk density of the solid formulation comprising an indolinone of formula I is greater than about 0.5 kg/L.

In preferred embodiments, the tap density of the solid formulation comprising an indolinone of formula I is from about 0.6 kg/L to about 0.7 kg/L or from about 0.5 kg/L to about 0.7 kg/L.

In other preferred embodiments, the tap density of the solid formulation comprising an indolinone of formula I is greater than about 0.5 kg/L.

Generally, bulk density is the weight of a unit volume of material. Bulk density is also known as “apparent density.” Bulk density is typically expressed as a weight:volume ratio, for example, kilograms per liter (Kg/L) or grams per cubic centimeter (g/cm²). Bulk desntiy of a material can be measured by techniques that are well known in the art, by measuring the weight and volume of the material. See the Examples section of this application for exemplary techniques for measurement of bulk density.

“Tap density” is also used to assess the properties of the formulations of the invention. Tap density can be measured by techniques that are well known in the art, and apparati for measuring tap density are commercially available. Briefly, the material is subjected to a series of “taps” that cause the material to be compacted, or reduced in volume. The density (weight/volume) of the “tapped” material is the tap density. Typically, the tapping is carried out until the tap density measurement has stabilized. For example, as shown in Comparative Example section of this application, material was tapped 500 times, volume was measured (volume measurement #1). The material was then tapped an additional number of times so that total number of taps was 1250, and then a second volume measurement (#2) was taken. If measurement #1 and #2 differed by greater than 2 milliliters, then the material was tapped an additional 1250 times.

In other preferred embodiments, the ratio of tap density to bulk density of the formulation is from about 1.10 to about 1.30. In still other preferred embodiments, the ratio of tap density to bulk density is from about 1.10 to about 1.30. In other preferred embodiments, the ratio of tap density to bulk density is from about 1.10 to about 1.33. In other preferred embodiments, the ratio of tap density to bulk density is from about 1.10 to about 1.25. In other preferred embodiments, the ratio of tap density to bulk density is from about 1.10 to about 1.20. In other preferred embodiments, the ratio of tap density to bulk density is from about 1.10 to about 1.15.

In another preferred embodiment, the formulation comprises an indolinone compound of formula I, wherein no more than 55% of the particles have a size less than 250 microns. In yet another embodiment, the formulation comprises an indolinone compound of formula I, wherein the mean particle size of the particles in the formulation is in the range of about 106-250 microns. In still other preferred embodiments, the formulation comprises an indolinone compound of formula I, wherein the mean particle size of the particles in the formulation is in the range of about 150-250 microns or in the range of about 250-350 microns. In another embodiment, the formulation comprises an indolinone compound of formula I, wherein no more than about 55% of the particles of the formulation have a size between about 106 and about 250 microns. In other embodiments, the formulation comprises an indolinone compound of formula I, wherein no more than about 50% or about 45% or about 40% or about 35% or about 30% of the particles of the formulation have a size between about 106 and about 250 microns. In still other preferred embodiments, the formulations having the afore-mentioned particle sizes comprise the malate salt of the compound of formula I. In yet other preferred embodiments, the formulations having the afore-mentioned particle sizes comprise 15-50% w/w of the malate salt of a compound of formula I; preferably 35-45% w/w of the malate salt of a compound of formula I; preferably 15-40% w/w of the malate salt of a compound of formula I. In still other preferred embodiments, the formulations having the afore-mentioned particle sizes comprise a compound of formula I, or a pharmaceutically acceptable salt thereof, and 1.5% w/w magensium stearate.

In a preferred embodiment, the formulation of the preferred embodiments of the present invention comprises 5-60% w/w of an indolinone of formula I, or a pharmaceutically acceptable salt thereof; preferably, 5-55% w/w of an indolinone of formula I, or a pharmaceutically acceptable salt thereof preferably, 10-60% w/w of an indolinone of formula I, or a pharmaceutically acceptable salt thereof; preferably, 15-50% w/w of an indolinone of formula I, or a pharmaceutically acceptable salt thereof; preferably, 35-45% w/w of an indolinone of formula I, or a pharmaceutically acceptable salt thereof; preferably, 39-43% w/w of an indolinone of formula I, or a pharmaceutically acceptable salt thereof; preferably, 10-40% w/w of an indolinone of formula I, or a pharmaceutically acceptable salt thereof; preferably, 20-50% w/w of an indolinone of formula I, or a pharmaceutically acceptable salt thereof; preferably, 38-42% w/w of an indolinone of formula I, or a pharmaceutically acceptable salt thereof; preferably, 38-41% w/w of an indolinone of formula I, or a pharmaceutically acceptable salt thereof; preferably, 39-41% w/w of an indolinone of formula I, or a pharmaceutically acceptable salt thereof; preferably, 10-45% w/w of an indolinone of formula I, or a pharmaceutically acceptable salt thereof; preferably, 15-40% w/w of an indolinone of formula I, or a pharmaceutically acceptable salt thereof; most preferably 40% w/w of an indolinone of formula I, or a pharmaceutically acceptable salt thereof.

In other preferred embodiments, the formulation of the present invention comprises 10-86% wIw of a diluent; preferably 10-80% w/w of a diluent; preferably 20-86% w/w of a diluent; preferably 30-80% w/w of a diluent; preferably 10-25% w/w of a diluent; preferably 25-50% w/w of a diluent; preferably 34-60% w/w of a diluent; preferably 34-77% w/w of a diluent; preferably 45-65% w/w of a diluent; preferably, 39-80% w/w of a diluent; preferably, 45-49% w/w of a diluent; preferably, 46-50% w/w of a diluent; preferably, 45-48% w/w of a diluent; preferably, 46-48% w/w of a diluent; most preferably, 47.5% w/w of a diluent.

In other preferred embodiments, the formulation of the preferred embodiments of the present invention comprises 2-20 w/w of a binder; preferably 2-10% w/w of a binder; preferably 5-20% w/w of a binder; preferably, 5-10% w/w of a binder; preferably, 3-6% w/w of a binder; preferably, 3-8% w/w of a binder; preferably, 4-6% w/w of a binder; preferably, 5-10% w/w of a binder; preferably 4-8% w/w of a binder; preferably 5-9% w/w of a binder; preferably 4-7% w/w of a binder; preferably 5-7% w/w of a binder; most preferably, 5% w/w of a binder.

In other preferred embodiments, the formulation of the preferred embodiments of the present invention comprises 2-20% w/w of a disintegrant; preferably 2-10% w/w of a disintegrant; preferably 5-20 w/w of a disintegrant; preferably, 5-10% w/w of a disintegrant; preferably 4-8% w/w of a disintegrant; preferably 5-8% w/w of a disintegrant; preferably, 3-7% w/w of a disintegrant; preferably, 3-6% w/w of a disintegrant; preferably, 4-6% w/w of a disintegrant; most preferably, 6% w/w of a disintegrant.

In other preferred embodiments, the formulation of the preferred embodiments of the present invention comprises 1-10% w/w of a lubricant; preferably, 0.1-2.5% w/w of a lubricant; preferably, 1-5% w/w of a lubricant; preferably 0.5-2% w/w of a lubricant; preferably, 1-2% w/w of a lubricant; preferably, 1-1.5% w/w of a lubricant; preferably, 1-2.5% w/w of a lubricant; preferably 1.3-1.7% w/w of a lubricant; preferably 1.4-1.8% w/w of a lubricant; preferably 1.3-1.6% w/w of a lubricant; preferably 1.4-1.6% w/w of a lubricant; most preferably 1.5% w/w of a lubricant.

In other preferred embodiments, the formulation of the preferred embodiments of the present invention comprises 15-60% w/w of an indolinone of formula I, or a pharmaceutically acceptable salt thereof, 25-75% w/w mannitol, 4-8% w/w croscaramellose sodium, 4-6% w/w povidone and 0.5-2% w/w magnesium stearate.

In still other preferred embodiments, the formulation of the preferred embodiments of the present invention comprises 30-50% w/w of an indolinone of formula I, or a pharmaceutically acceptable salt thereof, 35-60% w/w mannitol, 5-8% w/w croscaramellose sodium, 4-6% w/w povidone and 1-2% w/w magnesium stearate.

In a most preferred embodiment of the present invention, the formulation of the preferred embodiments of the present invention comprises 40% w/w of an indolinone of formula I, or a pharmaceutically acceptable salt thereof, 47.5% w/w mannitol, 6% w/w croscaramellose sodium, 5% w/w povidone and 1.5% w/w magnesium stearate.

In other preferred embodiments, the formulation of the preferred embodiments of the present invention comprises 10-16% w/w of an indolinone of formula 1, or a pharmaceutically acceptable salt thereof, 65-80% w/w mannitol, 5-10% w/w croscaramellose sodium, 4-8% w/w povidone and 1-2% w/w magnesium stearate.

In other preferred embodiments, the formulation of the preferred embodiments of the present invention comprises 15.2% w/w of an indolinone of formula I, or a pharmaceutically acceptable salt thereof, 72.7% w/w mannitol, 6% w/w croscaramellose sodium, 5.1% w/w povidone and 1% w/w magnesium stearate.

In other preferred embodiments, the formulation of the preferred embodiments of the present invention comprises 38-42% w/w of an indolinone of formula I, or a pharmaceutically acceptable salt thereof, 45-49% w/w mannitol, 4-8% w/w croscaramellose sodium, 3-7% w/w povidone and 1.3-1.7% w/w magnesium stearate.

In other preferred embodiments, the formulation of the preferred embodiments of the present invention comprises 39-43% w/w of an indolinone of formula I, or a pharmaceutically acceptable salt thereof, 46-50% w/w mannitol, 5-9% w/w croscaramellose sodium, 4-8% w/w povidone and 1.4-1.8% w/w magnesium stearate.

In other preferred embodiments, the formulation of the preferred embodiments of the present invention comprises 38-41% w/w of an indolinone of formula I, or a pharmaceutically acceptable salt thereof, 45-48% w/w mannitol, 4-7% w/w croscaramellose sodium, 3-6% w/w povidone and 1.3-1.6% w/w magnesium stearate.

In other preferred embodiments, the formulation of the preferred embodiments of the present invention comprises 39-43% w/w of an indolinone of formula I, or a pharmaceutically acceptable salt thereof, 46-50% w/w mannitol, 5-9% w/w croscaramellose sodium, 4-8% w/w povidone and 1.4-1.8% w/w magnesium stearate.

In other preferred embodiments, the formulation of the preferred embodiments of the present invention comprises 39-41% w/w of an indolinone of formula I, or a pharmaceutically acceptable salt thereof, 46-48% w/w mannitol, 5-7% w/w croscaramellose sodium, 4-6% w/w povidone and 1.4-1.6% w/w magnesium stearate.

In other preferred embodiments, the formulation of the preferred embodiments of the present invention comprises 39-43% w/w of an indolinone of formula I, or a pharmaceutically acceptable salt thereof, 46-50% w/w mannitol, 5-9% w/w croscaramellose sodium, 4-8% w/w povidone and 0.8-1.5% w/w magnesium stearate.

In other preferred embodiments, the formulation of the preferred embodiments of the present invention comprises 39-43% w/w of an indolinone of formula I, or a pharmaceutically acceptable salt thereof, 46-50% w/w mannitol, 5-9% w/w croscaramellose sodium, 4-8% w/w povidone and 0.8-1.2% w/w magnesium stearate.

Alternatively, the bulk density of the solid formulation comprising an indolinone of formula I is from about 2 to about 8 fold greater than the bulk density of the indolinone of formula I itself. In other preferred embodiments, the bulk density of the solid formulation comprising a malate salt of an indolinone of formula I is about 2 to about 8 fold greater than the bulk density of the malate salt of the indolinone of formula I itself. In more preferred embodiments, the bulk density of the formulation is about 3 to about 8 fold, or about 4 to about 8 fold , or about 5 to about 8 fold, or about 6 to about 8 fold greater than the bulk density of the indolinone of formula I (or the malate salt thereof) itself.

In other preferred embodiments, the bulk density of the solid formulation comprising a malate salt of an indolinone of formula I is at least about 2 fold greater than the bulk density of the malate salt of the indolinone of formula I itself. In more preferred embodiments, the bulk density of the formulation is at least about 3 fold, or at least about 4 fold greater than the bulk density of the indolinone of formula I (or the malate salt thereof) itself. In a more preferred embodiment, the bulk density of the formulation is at least about 5 fold greater than the bulk density of the indolinone of formula I (or the malate salt thereof) itself. In the most preferred embodiments, the bulk density of the formulation is at least about 6 fold or at least about 7 fold greater than the bulk density of the indolinone of formula I (or the malate salt thereof) itself.

In a most preferred embodiment of the present invention, the formulation does not comprise a flow enhancer (e.g., colloidal silicon dioxide, talc, and the like) or a surfactant (e.g., an ethylene oxide copolymer like PLURONIC™ F68 and the like).

Methods of Treatment

In preferred embodiments of the invention, the formulations are effective in treating or preventing an abnormal condition in a patient in need of such treatment. The patient is preferably a mammal and more preferably a human.

The term “preventing” as used herein refers to adiministering the formulation to a patient before the abnormal condition manifests itself in that patient.

The term “treating” as used herein refers to the method of the invention having a therapeutic effect and at least partially alleviating or abrogating the abnormal condition in the organism (e.g., patient).

The term “therapeutic effect” as used herein refers to inhibition of the abnormal condition. The term “therapeutic effect” also refers to the inhibition of factors causing or contributing to the abnormal condition. A therapeutic effect relieves to some extent one or more of the symptoms of the abnormal condition.

The term “mammal” as used herein preferably refers to the organisms of the class known as “mammalia”, such as mice, rats, rabbits, guinea pigs, goats, sheep, horses, cows, dogs, cats, monkeys, apes, humans, and the like; more preferably dogs, cats, monkeys, apes, humans, and the like; and most preferably humans.

The term “abnormal condition” refers to a function in the cells or tissues of a patient that deviates from normal functions in that patient. An abnormal condition can relate to cell proliferation (e.g., be a cell proliferative disorder) as described herein.

The term “cell proliferative disorder” as used herein refers to a disorder where an excess cell proliferation of one or more subset of cells in a multicellular organism occurs resulting in harm (e.g., discomfort or decreased life expectancy) to the multicellular organism. The excess cell proliferation can be determined by reference to the general population and/or by reference to a particular patient (e.g., at an earlier point in the patient's life). Hyper-proliferative cell disorders can occur in different types of animals and in humans, and produce different physical manifestations depending upon the affected cells. Hyper-proliferative cell disorders include without limitation cancers, blood vessel proliferative disorders, fibrotic disorders, autoimmune disorders, and the like. Cell proliferative disorders suitable for treatment in accordance with the present invention include without limitation cancers (e.g., erythroblastoma, glioblastoma, meningioma, astrocytoma, melanoma, myoblastoma, breast cancers, gastric cancers, ovarian cancers, renal cancers, hepatic cancers, pancreatic cancers, bladder cancers, thyroid cancers, prostate cancers, colorectal cancers, solid tumor cancers, colon cancer, brain cancer, blood cancers, bone cancers, liver cancer, kidney cancer, stomach cancer, lung cancer, Kaposi's sarcoma, non-small cell lung cancer, skin cancer, and the like, non-small cell lung cancers, and the like).

In reference to the treatment of abnormal conditions caused, in whole or in part, by a cell proliferative disorder, a therapeutic effect refers to one or more of the following: (a) reducing tumor size; (b) inhibiting (e.g, slowing or stopping) tumor metastasis; (c) inhibiting tumor growth; and (d) relieving to some extent one or more of the symptoms associated with the abnormal condition.

Thus, the present invention features methods of preventing or treating an abnormal condition in a patient in need of treatment comprising orally administering a formulation comprising an indolinone, a binder, a disintegrant, a lubricant and a diluent to said patient. Preferably, the indolinone is an indolinone of Formula I, and pharmaceutically active salts, prodrugs, derivatives, and analogs thereof. In a preferred embodiment, the formulation lacks a surfactant.

In preferred embodiments of a method of preventing or treating an abnormal condition in a patient in need of treatment with an oral formulation, the indolinone is solubilized by combining with a molar equivalent of an acid solution. Preferably, the acid solution is selected from the group consisting of hydrochloric acid, sulfuric acid, formic acid, lactic acid, malic acid, maleic acid, succinic acid, acetic acid, methane sulfonic acid, benzene sulfonic acid, phosphoric acid, and the like, and suitable combinations of two or more hereof.

In yet other preferred embodiments of a method of preventing or treating an abnormal condition in a patient in need of treatment with an oral formulation, one or more polyhydroxycarbyl compounds are added to the formulation. The one or more polyoxyhydrocarbyl compounds is selected from the group consisting of polyethylene glycol 300, polyethylene glycol 400, propyleneglycol, glycerin, and the like, and suitable combinations of two or more hereof. Preferably, the one or more polyoxyhydrocarbyl compounds is polyethylene glycol 300.

In other preferred embodiments of a method of preventing or treating an abnormal condition in a patient in need of treatment with an oral formulation, a buffer is added to the formulation. The buffer pH is three pH units lower than the pkb of said ionizable substituted indolinone. Preferably, the buffer has a molarity between 0.01 M and 0.1 M, and is selected from the group consisting of acetate, citrate, phosphoric acid buffer, ascorbate, hydrochloric acid buffer, Tris-HCl buffer, and the like, and suitable combinations of two or more hereof. Alternatively, the buffer is selected from the group consisting of sodium phosphate, sodium carbonate, sodium hydroxide, glutamate, glycine, Tris base buffers, and the like, and suitable combinations of two or more hereof. Preferably, the buffer is sodium phosphate buffer.

In other preferred embodiments of a method of preventing or treating an abnormal condition in a patient in need of treatment with an oral formulation, the formulation also contains a pharmaceutically acceptable preservative. Preferably, the preservative is selected from the group consisting of benzyl alcohol, methyl paraben, ethyl paraben, phenol, and the like, and suitable combinations of two or more hereof. Most preferably, the preservative is benzyl alcohol.

In other preferred embodiments of a method of preventing or treating an abnormal condition in a patient in need of treatment with an oral formulation, the formulation also contains an antioxidant. Preferably, the antioxidant is selected from the group consisting of sodium meta-bisulfite, EDTA, ascorbic acid, benzyl alcohol, and the like, and suitable combinations of two or more hereof, and preferably is benzyl alcohol.

In yet other preferred embodiments of a method of preventing or treating an abnormal condition in a patient in need of treatment with an oral formulation, the patient is a mammal, preferably a human.

Further, the present invention features methods of preventing or treating an abnormal condition in a patient in need of treatment comprising orally administering to the patient a formulation comprising an ionizable substituted indolinone, one or more pharmaceutically acceptable surfactants, and one or more pharmaceutically acceptable oils.

Other features and advantages of the invention will be apparent from the following description of the preferred embodiments and from the claims.

Dosage

Compounds, combinations, and pharmaceutical compositions and formulations suitable for use in the present invention include compositions wherein the active ingredients are contained in an amount sufficient to achieve the intended purpose; i.e., the modulation of protein kinase (PK) activity or the treatment or prevention of a PK-related disorder.

The above referenced protein kinase related disorder is selected from the group consisting-of an EGFR related disorder, a PDGFR related disorder, an IGFR related disorder and a flk related disorder.

The above referenced protein kinase related disorder is a cancer selected from the group consisting of squamous cell carcinoma, sarcomas such as Kaposi's sarcoma, astrocytoma, glioblastoma, lung cancer, bladder cancer, colorectal cancer, gastrointestinal cancer, head and neck cancer, melanoma, ovarian cancer, prostate cancer, breast cancer, small-cell lung cancer, leukemia and glioma in a further aspect of this invention.

The above referenced protein kinase related disorder is selected from the group consisting of diabetes, a hyper-proliferation disorder, von Hippel-Lindau disease, restenosis, fibrosis, psoriasis, osteoarthritis, rheumatoid arthritis, an inflammatory disorder, mastocytosis and angiogenesis in yet another aspect of this invention.

Additional disorders which may be treated or prevented using the compounds of this invention are immunological disorders such as autoimmune disease (AIDS) and cardiovasular disorders such as atherosclerosis.

More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the PK activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact dosage can be chosen by the individual physician in view of the patient's condition. (See e.g., Fingl, et al., 1975, in “The Pharmacological Basis of Therapeutics”, Ch. 1 p.1).

Therapeutic compounds should be more potent in inhibiting receptor tyrosine kinase activity than in exerting a cytotoxic effect. A measure of the effectiveness and cell toxicity of a compound can be obtained by determining the therapeutic index; i.e., IC₅₀/LD₅₀. IC₅₀, the dose required to achieve 50% inhibition, can be measured using standard techniques such as those described herein. LD₅₀, the dosage which results in 50% toxicity, can also be measured by standard techniques as well(Mossman, 1983, J. Immunol. Methods, 65:55-63), by measuring the amount of LDH released (Korzeniewski and Callewaert, 1983, J. Immunol. Methods, 64:313; Decker and Lohmann-Matthes, 1988, J. Immunol. Methods, 115:61), or by measuring the lethal dose in animal models. Compounds with a large therapeutic index are preferred. Thus, in one aspect of the invention, a preferred dosage of the compounds, agents, combinations, and pharmaceutical compositions contemplated for use in the invention requires the therapeutic index of each active component to be greater than 2, preferably at least 10, more preferably at least 50.

Dosage amount and interval may be adjusted individually to provide plasma levels of the active species, which are sufficient to maintain the kinase modulating effects. These plasma levels are referred to as minimal effective concentrations (MECs). The MEC will vary for each compound but can be estimated from in vitro data; e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

In general, a “therapeutically effective amount” refers to that amount of an agent or its metabolite which is effective to prevent, alleviate, reduce or ameliorate symptoms of disease and/or the undesired side effects attributable to treatment of disease with another agent or its metabolite, or to prolong the survival of the patient being treated. More particularly, in reference to the treatment of cancer, a therapeutically effective amount refers to that amount which has the effect of (1) reducing the size of (or preferably eliminating) the tumor; (2) inhibiting (that is, slowing to some extent, preferably stopping) tumor metastasis; (3) inhibiting to some extent (that is slowing to some extent, preferably stopping) tumor growth; and/or, (4) relieving to some extent (or preferably eliminating) one or more symptoms associated with the cancer and/or one or more undesired side effects attributable to treatment of the cancer with another agent or its metabolite. Non-limiting examples of therapeutically effective amounts of particular agents and compounds contemplated for use in the present invention are further described below.

In addition to the above general definition, by a “therapeutically effective amount” of an agent is meant any amount administered in any manner and in any treatment regime as may be currently recognized in the medical arts or as may come about as the result of future developments regarding the use of these agents.

A “treatment regime” refers to specific quantities of the ionizable substituted indolinone contemplated for use in this invention) administered at set times in a set manner over an established time period.

When referring to “set times” of administration within a treatment regime, “consecutive days” means consecutive calendar days; i.e., Monday, Tuesday, Wednesday, etc. “Staggered” days means calendar days with other calendar days between them, e.g., without limitation, Monday, Wednesday, Saturday, etc.

Furthermore, with regard to a “therapeutically effective amount” of an ionizable substituted indolinone, the phrase refers to an amount of the compound sufficient to inhibit the growth, size and vascularization; i.e., angiogenesis and/or vasculogenesis, of tumors during the “recovery” periods, i.e., the periods in a treatment regime when no other chemotherapeutic agent is being administered to a patient.

The compounds of the preferred embodiments of the present invention may be administered in doses ranging from about 1 mg/m² to about 3000 mg/m². In a presently preferred embodiment, the dosage is between about 50 mg/m² and about 2400 mg/m². In another preferred embodiment, therapeutically effective amounts of indolinone composition comprise from about 50 to about 800 mg/m². Of course, the dose would depend on a number of factors, including patient specific factor, e.g., weight, dosing regimen (e.g., frequency, route of administration, effect of food) etc.

In a presently preferred embodiment, the indolinone composition dose is administered during rest periods when no other agent is being administered to a patient.

Tablets

Methods of making tablets are known in the art. The three basic methods for the preparation of compressed tablets or capsules are the wet granulation method, the dry granulation method and direct compression (tablets). (Ansel et al., “Pharmaceutical Dosage Forms and Drug Delivery Systems” 1995, Williams and Wilkins, which is incorporated by reference in its entirety).

Wet granulation is a widely employed method for the production of compressed tablets or capsules. The steps required in the the preparation of tablets or capsules by this method may be separated as follows: (1) weighing and blending the ingredients (2) preparing the wet granulation (3) screening the damp mass into pellets or granules (4) drying (5) dry screening (6) lubrication and blending and (7) tableting by compression or encapsulating. In weighing and blending the active ingredient and any filler, disintegrating agent required in the formulation are weighed and mixed thoroughly. The total amount of the disintegrant is not always added to the drug-diluent mixture, but a portion is reserved for later addition with the lubricant to the prepared granulation of the drug. Granulation is accomplished by adding a liquid binder or an adhesive to the powder mixture, passing the wetted mass through a screen of the desired mesh size, drying the granulation and then passing through a second screen of smaller mesh size to reduce further the size of the granules. Generally the wet granulation is pressed through a mesh screen. After all of the material has been converted into granules, the granulation is spread evenly on large pieces of paper and dried. After drying, a dry lubricant is generally added to the granulation so that each granule is covered with lubricant. The formulation is then pressed into tablets or encapsulated.

In the dry granulation method, the granulation is formed not by moistening or adding a binding agent to the powdered drug mixture byt by compressing large masses of the mixture and subsequently crushing and sizing these pieces into smaller granules. By this method either the active ingredient or the diluent should have cohesive properties in order for the large masses to be formed. After weighing and mixing the ingredients the powder is “slugged” or compressed into large flat tablets or pellets. The slugs are broken up by hand or by a mill and passed through a screen of desired mesh size for sizing. Lubricant is added and tablets are prepared by compression or the drug mixture is encapsulated.

Packaging

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

### SYNTHESIS EXAMPLES

The compounds of this invention, as well as the precursor 2-oxindoles and aldehydes, may be readily synthesized using techniques well known in the chemical arts. The syntheses of the compounds of the preferred embodiments of the present invention is disclosed in U.S. Ser. No. 10/076,140, filed Feb. 15, 2002, PCT Application No. PCT/US02/04407, and published PCT application WO 01/60814; and U.S. Ser. No. 10/281,985, filed Aug.13, 2002, claiming priority to U.S. Ser. No. 60/312,353, filed Aug. 15, 2001; all of which are incorporated herein by reference. Yet, it will be appreciated by those skilled in the art that other synthetic pathways for forming the compounds of the invention are available and that the following is offered by way of example and not limitation.

### FORMULATION EXAMPLES

Generally, the formulations of the preferred embodiments of the present invention are prepared by combining the active pharmaceutical ingredient (API) with one or more pharmaceutically acceptable diluents, one or more pharmaceutically acceptable binders, one or more pharmaceutically acceptable disintegrants and one or more pharmaceutically acceptable lubricants.

Method for Making a Granular Composition

1. Mix all ingredients, except magnesium stearate and 50% croscarmellose sodium in a high shear granulator.

2. Granulate using purified water as the granulating fluid.

3. Dry granules in a fluid bed granulator.

4. Mill dried granules with an oscillating sieve to appropriate granule size.

5. Blend the sieved granule with the remaining croscarmellose sodium (50%) in an appropriate size.

6. Add magnesium stearate and blend.

### Example 1

Composition of 5-(5-fluoro-2-oxo-1,2-dihydro-indol-3-
ylidenemethyl)-2,4-dimethyl-1H-pyrrole-3-carboxylic
acid (2-diethylamino-ethyl)-amide hard gelatin capsules

| | | Amount | Amount | Amount |
|---|---|---|---|---|
| | Concentration in | in 50 mg | in 75 mg | in 200 mg |
| | Granulation | Capsule | Capsule | Capsule |
| Ingredient Name | (% w/w) | (mg) | (mg) | (mg) |
| API | 65.0 | 50.0 | 75.0 | 200.0 |
| Mannitol | 23.5 | 18.1 | 27.2 | 72.4 |
| Croscaramellose | 6.0 | 4.6 | 6.9 | 18.4 |
| Sodium^{e} | | | | |
| Povidone (K-25) | 5.0 | 3.8 | 5.7 | 15.2 |
| Magnesium | 0.5 | 0.38 | 0.57 | 1.52 |
| Stearate | | | | |
| Capsule | — | Size 1 | Size 3 | Size 0 |

In Example 1, the free base form of the compound was used. The bulk density of the granular composition of the formulation used to make a 50 mg capsule was 0.44 kg/L and the tap density was 0.60 kg/L. The bulk density of the granular composition of the formulation used to make a 75 mg capsule was 0.46 kg/L and the tap density was 0.63 kg/L. The ratio of tap to bulk density for both formulations was 1.36 kg/L.

### Example 2

Composition of 5-(5-fluoro-2-oxo-1,2-dihydro-indol-3-
ylidenemethyl)-2,4-dimethyl-1H-pyrrole-3-carboxylic acid
(2-diethylamino-ethyl)-amide L-malate hard gelatin capsules

| | Concentration | |
|---|---|---|
| | in Granulation | Amount in 50 mg |
| Ingredient Name/Grade | (% w/w) | Capsule (mg) |
| API | 75.0 | 66.800^{c} |
| Mannitol | 13.5 | 12.024 |
| Croscaramellose Sodium^{e} | 6.0 | 5.344 |
| Povidone (K-25) | 5.0 | 4.453 |
| Magnesium Stearate | 0.5 | 1.445 |
| Capsule | — | Size 3 |

### Example 3

Composition of 5-(5-fluoro-2-oxo-1,2-dihydro-indol-
3-ylidenemethyl)-2,4-dimethyl-1H-pyrrole-3-carboxylic acid
(2-diethylamino-ethyl)-amide L-malate hard gelatin capsules

| | | Amount | Amount | Amount |
|---|---|---|---|---|
| | Concentration in | in 25 mg | in 50 mg | in 100 mg |
| Ingredient | Granulation | Capsule | Capsule | Capsule |
| Name/Grade | (% w/w) | (mg) | (mg) | (mg) |
| API^{a} | 40.0 | 33.400^{d} | 66.800^{c} | 133.6^{b} |
| Mannitol | 47.5 | 39.663 | 79.326 | 158.652 |
| Croscaramellose | 6.0 | 5.010 | 10.020 | 20.04 |
| Sodium^{e} | | | | |
| Povidone (K-25) | 5.0 | 4.175 | 8.350 | 16.700 |
| Magnesium | 1.5 | 1.252 | 2.504 | 5.008 |
| Stearate | | | | |
| Capsule | — | Size 3 | Size 1 | Size 0 |

^{a}Drug substance quantity required for the batch will be ajusted to have 100% of labeled strength for capsules. Appropriate adjustment will be made to mannitol quantity to keep the same fill weight for each strength.
^{b}Quantity equivalent to 100 mg free base.
^{c}Quantity equivalent to 50 mg free base.
^{d}Quantity equivalent to 25 mg free base.
^{e}Half intraganular half extragranular.

The bulk density of the L-malate salt 5-(5-fluoro-2-oxo-1,2-dihydro-indol-3-ylidenemethyl)-2,4-dimethyl-1H-pyrrole-3-carboxylic acid (2-diethylamino-ethyl)-amide, by itself, was measured to be between about 0.11±0.1. The bulk density of a formulation batch (different than the batches discussed below in the Comparative Example) was measured to be about 0.68 kg/L for the 50 mg capsule, and the tap density was about 0.81 kg/L. For the 25 mg capsules, the bulk density was about 0.64 kg/L and the tap density was about 0.8 kg/L. Therefore, the ratio of the bulk density of the formulation to the density of the L-malate salt is about 0.68/0.11=6.81 for the 50 mg capsules, and 0.64/0.11=5.81 for the 25 mg capsules.

### Example 4

Composition of SU011248 L-Malate Salt Drug Product:
12.5 mg Hard Gelatin Capsules

| | 12.5-mg Capsule | |
|---|---|---|
| | Concentration | Amount in |
| | in Granulation | 12.5-mg Capsule |
| Ingredient Name/Grade | (% w/w) | (mg) |
| Formulation Code | J-010398-AC | J-010398-AC-00 |
| SU011248 L-malate salt^{a} | 15.2 | 16.70^{b} |
| Mannitol NF | 72.7 | 80.00 |
| Croscarmellose sodium NF | 6.0 | 6.60 |
| Povidone (K-25) USP | 5.1 | 5.60 |
| Magnesium stearate NF | 1.0 | 1.10 |
| Total Fill Weight | 100 | 110.0 |
| Capsule | — | Swedish Orange, Size 4 |

^{a}Drug substance quantity required for the batch will be adjusted to contain 100% of labeled strength. Appropriate adjustment will be made to mannitol quantity to keep the same fill weight for each strength.
^{b}Quantity equivalent to 12.5 mg free base

The bulk density of this formulation was aobout 0.67 kg/L and the tap density was 0.77 kg/L. The size distribution of the particles are as follows:

| Particle Size (μm) | Percentage |
|---|---|
| >1000 | 0.26 |
| 1000-710 | 6.50 |
| 710-500 | 5.1 |
| 500-250 | 19.0 |
| 250-106 | 54.0 |
| <106 | 15.2 |

### Example 5

### Formulation for 5-[(Z)-(5-fluoro-2-oxo-1,2-dihydro-3H-indol-3-ylidene)methyl]-N-[2-hydroxy-3-morpholin-4-ylpropyl]-2,4-dimethyl-1H-pyrrole-3-carboxamide

5-[(Z)-(5-fluoro-2-oxo-1,2-dihydro-3H-indol-3-ylidene)methyl]-N-[2-hydroxy-3-morpholin-4-ylpropyl]-2,4-dimethyl-1H-pyrrole-3-carboxamide is formulated as as the maleate salt. The compound is formulated in the same fashion as described above in the section entitled “Method for Making a Granular Composition” and in Examples 1-4. The compound is formulated as either the (R) isomer, the (S) isomer or as mixtures of both isomers.

The maleate salt of this compound was determined to have a bulk density of about 0.05-0.07 Kg/l.

### COMPARATIVE EXAMPLE

A capsule containing a formulation comprising 75% w/w of the malate salt of 5-(5-fluoro-2-oxo-1,2-dihydro-indol-3-ylidenemethyl)-2,4-dimethyl-1H-pyrrole-3-carboxylic acid (2-diethylamino-ethyl)-amide was developed. See Table 1, below. During the capsule production using this amount of the API, however, excessive sticking problems were observed during the capsule filling process. The sticking problems occurred in the hopper, filling heads and other moving parts of the capsule filling machine. The sticking problems necessiatated halting the capsule filling process several times to clean machine parts.

An improved formulation was developed as shown in Table 1, below. The new formulation comprises 40% w/w 5-(5-fluoro-2-oxo-1,2-dihydro-indol-3-ylidenemethyl)-2,4-dimethyl-1H-pyrrole-3-carboxylic acid (2-diethylamino-ethyl)-amide L-malate and 1.5% w/w magensium stearate. The improved formulation did not exhibit the sticking problems observed with the 75% w/w formulation.

TABLE 1
Comparison of the 75% w/w and the 40% w/w formulation*

| Ingredient Name/ | concentration | Ingredient Name/ | concentration |
|---|---|---|---|
| Grade | (% w/w) | Grade | (% w/w) |
| API | 75.00 | API | 40.00 |
| Mannitol | 13.50 | Mannitol | 47.50 |
| Sodium | 3.00 | Sodium | 3.00 |
| croscarmelose | | croscarmelose (in) | |
| (in) | | | |
| Povidone K25 | 5.00 | Povidone K25 (in) | 5.00 |
| (in) | | | |
| Sodium | 3.00 | Sodium | 3.00 |
| croscarmelose | | croscarmelose (out) | |
| (out) | | | |
| Magnesium | 0.50 | Magnesium | 1.50 |
| Stearate (out) | | Stearate (out) | |

*The reduction of API was compensated for by an increase in mannitol amount.

Granulation and Blending Procedures

Three batches were produced: two using 150 g API and one using 200 g API.

For wet granulation, a high shear granulator (Key international KG 5) equipped with a 3 L bowl was used. The 200 g API batch (500 g dry mixture) filled about 45% of the bowl volume.

The API and the intragranular excipients (addition order: mannitol, povidone and croscaramellose sodium) were mixed into the high shear granulator for about 2 minutes using impeller speed of about 300 rpm and chopper speed of about 4,000 rpm. The residual water content (L.O.D.) of the dry mixture was measured on a representative sample and expressed as percent loss of mass upon drying of the sample (test conducted using a thermobalance with drying temperature 110° C., until sample constant weight is reached).

Water was added through a funnel to the mixture; impeller speed was about 400 rpm and chopper speed was about 5000-6000 rpm. Water was added and material was kneaded until the granules were wet but not sticky. The ordinary skilled artisan would be able to ascertain the point at which the granules are wet but not sticky.

The amounts of water added and granulation times are summarized in the following table.

TABLE 2
Details of the Granulation Process

| | | | Water % | |
|---|---|---|---|---|
| API Amount | Dry mixture | Water | (on whole | Granulation |
| (g) | L.O.D. | amount | formulation) | time |
| 150 | 1.65% | 90 | 24.0 | 6′30″ |
| | | overwetted | | |
| 150 | 1.57% | 70 | 18.7 | 8′ |
| 200 | 1.41% | 92.2 | 18.4 | 8′ |

The drying process was conducted on a Uni-Glatt fluid bed dryer with inlet air temperature at 60° C., until an outlet air temperature of 40° C. was reached. The L.O.D. evaluation was done and the drying process stopped if a value equal to or less than that of the starting dry mixture was obtained.

The drying processes for the second and third batches were conducted with flap at 25-30% and lasted 19 and 22 minutes, respectively. The L.O.D. values at the end of the process were below the required limit.

The drying process was stopped when the L.O.D. was below 2.5%.

Dry granules were sized through mill (fluidair granulmnill junior) equipped with 1 mm screen (round holes); the process was conducted with mill speed of 1500 rpm. At the end of the process, the L.O.D. was recorded and the values were inside the limits proposed.

For each granulation, the bulk and tap density and particle size distribution were recorded; for particle size distribution determination, Sonic Sieve Sifting equipment was used. See below for representative values for tap density and particle size distribution.

The granules from the batches that were not overwetted were combined and a final blend of 746.3 g was obtained. L.O.D. and density measurements and particle size distribution test were performed on the final blend. See below for data.

Capsule Filling Procedures

Manual

Using the above final blend, capsules of 25, 50 and 100 mg (calculated based on free base) were prepared.

The capsules were filled by hand using a volumetric filling head from a Zanasi AZ5 machine. Before starting encapsulation, twenty dosing weights were recorded to evaluate correct set up of the filling head.

During the filling, the dosing weight was periodically checked to guarantee as much uniformity as possible. Capsules and tooling were of size 3 for 25 mg (calculated based on free base), of size 1 for 50 mg and of size 0 for 100 mg.

Automatic

An automatic filling machine was equipped with size 3 dies on the feeding system and with size one holder on the capsule disc, to prepare 50 mg capsules. The operating speed was about 3000 capsules/hour; set up of the four tamping systems was at 20 mm (lowest pressure).

Results and Discussion

Granulation

The following tables report the densities and particle size distribution values for the two batches used to prepare the final blend.

TABLE 3
Density values for granulations

Batch

| | 2204-007 | 2204-014 | Final mixture |
|---|---|---|---|
| Bulk density | 0.61 | 0.62 | 0.63 |
| (g/mL) | | | |
| Tapped density | 0.73 | 0.75 | 0.77 |
| (g/mL) | | | |

TABLE 4
Particle size distribution values for granulations

| | Batch | | |
|---|---|---|---|
| | 2204-007 | 2204-014 | Final mixture |
| Mesh | % retained | % retained | % retained |
| 20 | 0.47 | 0.19 | 0.38 |
| 40 | 17.57 | 11.59 | 11.30 |
| 60 | 33.62 | 20.23 | 23.17 |
| 80 | 20.51 | 21.75 | 19.94 |
| 100 | 7.31 | 12.92 | 10.83 |
| 200 | 13.49 | 24.69 | 19.28 |
| fines | 7.22 | 8.64 | 10.64 |

The two granulation batches showed good densities and flowed very well.

Tap, Bulk Density and Particle Size Distribution Determinations

The tap and bulk density determinations are performed as follows:

(a) A 250 mL glass cylinder is filled with formulation granules to the 100-200 mL volume mark.

(b) The mass of the cyclinder is recorded and the bulk density is determined by calculating the ratio of the mass of the formulation granules to the volume of the formulation granules.

(c) The cylinder is then put into a tapping apparatus and the volume is and recorded after 10; 500 and 1250 taps.

(d) The ratio between the mass of the formulation granules and its volume after 1250 taps is the tap density.

(e) If the difference of volume after 500 and 1250 taps is higher than 2 mL, another 1250 taps are applied before reading the volume once again and calculating the tap density.

The particle size distributions are determined by using sieves (1000, 710, 500, 250, 106 microns) and a sieving apparatus which vibrates for a specified period of time (e.g., 3 minutes).

Capsule Filling

The final mixture obtained was used to fill manually 25 capsules of 25 mg (calculated based on free base) using a filling head from Zanasi AZ5 capsule filling machine, equipped with size 3 doser. The theoretical filling weight was 83.3 mg; the average weight of empty size 3 shells was 48.7 mg and the filled capsules average weight was 131.0 mg.

First, 550 capsules of 25 mg (granules batch 2204-014) were filled. For these capsules, the average weight of empty shells was 48.7 mg; the average weight of filled capsules was 130.1 mg. Eighty capsules of 50 mg (fill weight 166.6 mg, granules batch 2204-014) were prepared using size 1 shells having an average weight of 74.4 mg; the average weight of filled capsules was 241.2 mg. Fifty capsules of 100 mg (fill weight 333.2 mg, granules batch 2204-014) were prepared using size 0 shells having an average weight of 95.4 mg; the average weight of filled capsules was 428.0 mg.

The results of the automatic capsule filling (granules from batch 2204-014) test were favorable, even if the dies used (the smallest available) overshot the target filling weight.

It was possible to obtain a minimum filling weight of about 181.5 mg (theoretical 166.6 mg for 50 mg dose) and the uniformity of weight obtained was excellent (average weight relative standard deviation (CV)<1.0%) indicating very good flowability of the mixture. In other preferred embodiments, the CV may be from about 1 to about 6%; from about 6-4%; preferably from about 2 to about 4%; more preferably from about 1 to about 3%; most preferably <1%. About 3500 capsules were produced.

What is claimed is:

1. A solid formulation, said formulation comprising 5-60% w/w of an indolinone of formula I: 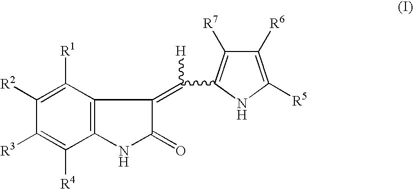
wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂)ₙR¹² or —NHCH₂CH(OH)CH₂R₁₂;
R¹¹ is selected from the group consisting of hydrogen and alkyl;
R¹² is selected from the group consisting of —NR¹³R¹⁴, —N⁺(O⁻)R¹³R¹⁴, —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4; or
pharmaceutically active salts of the compound of formula I; and
a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants.

2. The formulation of claim 1, wherein the salt of said indolinone is the malate salt.

3. The formulation of claim 1, wherein the salt of said indolinone is the maleate salt.

4. The formulation of claim 1, wherein the salt of said indolinone is the L-malate salt of

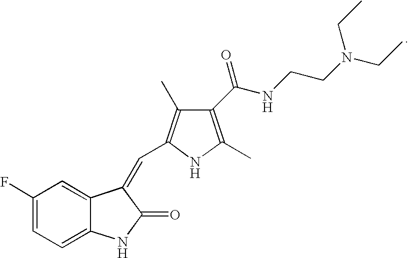

5. The formulation of claim 1, wherein the salt of said indolinone is the maleate salt of

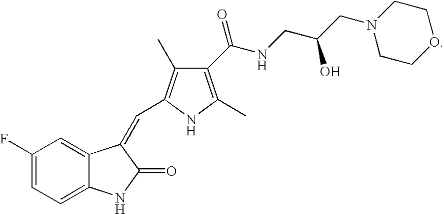

6. The formulation of claim 1, wherein the salt of said indolinone is the maleate salt of

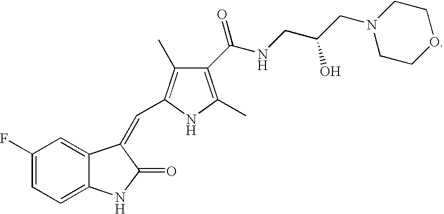

7. The formulation of claim 1, wherein the salt of said indolinone is a mixture of the maleate salt of

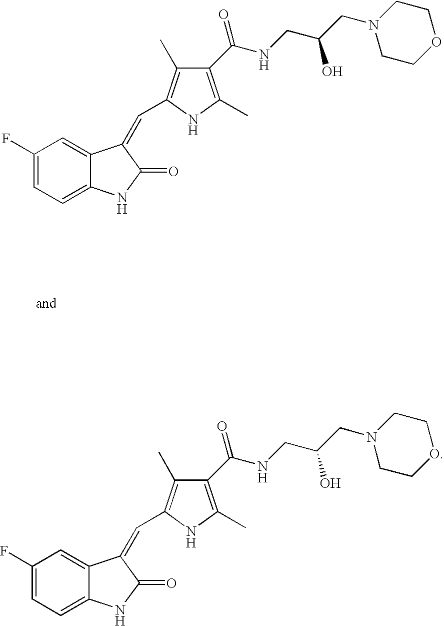

8. The formulation of claim 1, wherein each of said one or more pharmaceutically acceptable diluents is selected from the group consisting of pregelatinized starch, lactose monohydrate, lactose monohydrate regular grade, mannitol, calcium phosphate and microcrystalline cellulose.

9. The formulation of claim 1, wherein each of said one or more pharmaceutically acceptable binders is selected from the group consisting of polyvinylpyrrolidone, hydroxypropylmethyl cellulose, hydroxypropylcellulose and starch.

10. The formulation of claim 1, wherein each of said one or more pharmaceutically acceptable disintegrants is selected from the group consisting of crosscarmellose sodium, sodium starch glycolate, crospovidone, and starch.

11. The formulation of claim 1, wherein each of said one or more pharmaceutically acceptable lubricants is selected from the group consisting of magnesium stearate, sodium stearyl fumarate, glyceryl behenate and stearic acid.

12. The formulation of claim 1, wherein the amount of indolinone is from 5-55% w/w.

13. The formulation of claim 1, wherein the amount of indolinone is from 10-60% w/w.

14. The formulation of claim 1, wherein the amount of indolinone is from 15-50% w/w.

15. The formulation of claim 1, wherein the amount of indolinone is from 35-45% w/w.

16. The formulation of claim 1, wherein the amount of indolinone is from 39-43% w/w.

17. The formulation of claim 1, wherein the amount of indolinone is from 10-60% w/w.

18. The formulation of claim 1, wherein the amount of indolinone is from 10-40% w/w.

19. The formulation of claim 1, wherein the amount of indolinone is from 20-50% w/w.

20. The formulation of claim 1, wherein the amount of indolinone is from 38-42% w/w.

21. The formulation of claim 1, wherein the amount of indolinone is from 38-41% w/w.

22. The formulation of claim 1, wherein the amount of indolinone is from 39-41% w/w.

23. The formulation of claim 1, wherein the amount of indolinone is from 10-45% w/w.

24. The formulation of claim 1, wherein the amount of indolinone is from 15-40% w/w.

25. The formulation of claim 1, wherein the amount of diluent is from 10-80% w/w.

26. The formulation of claim 1, wherein the amount of diluent is from 20-86% w/w.

27. The formulation of claim 1, wherein the amount of diluent is from 30-80% w/w

28. The formulation of claim 1, wherein the amount of diluent is from 10-25% w/w.

29. The formulation of claim 1, wherein the amount of diluent is from 25-50% w/w.

30. The formulation of claim 1, wherein the amount of diluent is from 34-60% w/w.

31. The formulation of claim 1, wherein the amount of diluent is from 34-77%.

32. The formulation of claim 1, wherein the amount of diluent is from 45-65% w/w.

33. The formulation of claim 1, wherein the amount of diluent is from 39-80% w/w

34. The formulation of claim 1, wherein the amount of diluent is from 45-49% w/w.

35. The formulation of claim 1, wherein the amount of diluent is from 46-50% w/w.

36. The formulation of claim 1, wherein the amount of diluent is from 45-48% w/w.

37. The formulation of claim 1, wherein the amount of diluent is from 46-48% w/w.

38. The formulation of claim 1, wherein the amount of binder is from 2-10% w/w.

39. The formulation of claim 1, wherein the amount of binder is from 5-20% w/w.

40. The formulation of claim 1, wherein the amount of binder is from 5-10% w/w.

41. The formulation of claim 1, wherein the amount of binder is from 3-6% w/w.

42. The formulation of claim 1, wherein the amount of binder is from 3-8% w/w.

43. The formulation of claim 1, wherein the amount of binder is from 4-6% w/w.

44. The formulation of claim 1, wherein the amount of binder is from 5-10% w/w.

45. The formulation of claim 1, wherein the amount of binder is from 4-8% w/w.

46. The formulation of claim 1, wherein the amount of binder is from 5-9% w/w.

47. The formulation of claim 1, wherein the amount of binder is from 4-7% w/w.

48. The formulation of claim 1, wherein the amount of binder is from 5-7% w/w.

49. The formulation of claim 1, wherein the amount of disintegrant is from 2-10% w/w.

50. The formulation of claim 1, wherein the amount of disintegrant is from 5-20 w/w.

51. The formulation of claim 1, wherein the amount of disintegrant is from 5-10% w/w.

52. The formulation of claim 1, wherein the amount of disintegrant is from 4-8% w/w.

53. The formulation of claim 1, wherein the amount of disintegrant is from 5-8% w/w.

54. The formulation of claim 1, wherein the amount of disintegrant is from 3-7% w/w.

55. The formulation of claim 1, wherein the amount of disintegrant is from 3-6% w/w.

56. The formulation of claim 1, wherein the amount of disintegrant is from 4-6% w/w.

57. The formulation of claim 1, wherein the amount of lubricant is from 1-10% w/w.

58. The formulation of claim 1, wherein the amount of lubricant is from 0.1-2.5% w/w.

59. The formulation of claim 1, wherein the amount of lubricant is from 1-5% w/w.

60. The formulation of claim 1, wherein the amount of lubricant is from 0.5-2% w/w.

61. The formulation of claim 1, wherein the amount of lubricant is from 1-2% w/w.

62. The formulation of claim 1, wherein the amount of lubricant is from 1-1.5% w/w.

63. The formulation of claim 1, wherein the amount of lubricant is from 1-2.5% w/w.

64. The formulation of claim 1, wherein the amount of lubricant is from 1.3-1.7% w/w.

65. The formulation of claim 1, wherein the amount of lubricant is from 1.4-1.8% w/w.

66. The formulation of claim 1, wherein the amount of lubricant is from 1.3-1.6% w/w.

67. The formulation of claim 1, wherein the amount of lubricant is from 1.4-1.6% w/w.

68. The formulation of claim 1, wherein said diluent is mannitol.

69. The formulation of claim 1, wherein said binder is polyvinylpyrrolidone.

70. The formulation of claim 1, wherein said disintegrant is crosscaramellose sodium.

71. The formulation of claim 1, wherein said lubricant is magnesium stearate.

72. The formulation of claim 1, wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen and alkyl;
R⁶ is —C(O)R¹⁰ wherein R¹⁰ is —NR¹¹(CH₂)ₙR¹² wherein:
R¹¹ is hydrogen or lower unsubstituted alkyl;
n is 2 or 3; and
R¹² is selected from the group consisting of —NR¹³R¹⁴,—N⁺(O⁻)R¹³R¹⁴, and —N(OH)R¹³;
R⁷ is selected from the group consisting of hydrogen, alkyl, aryl and heteroaryl;
R⁸ and R⁹ are independently selected from the group consisting of hydrogen, alkyl and aryl;
R¹³ and R¹⁴ are independently selected from the group consisting of hydrogen, alkyl, lower alkyl substituted with hydroxy, alkylamino, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴; and
r is 1,2,3, or 4.

73. The formulation of claim 1, wherein R⁶ is —C(O)R¹⁰ wherein R¹⁰ is —NHCH₂CH(OH)CH₂R₁₂, wherein R₁₂ is selected from the group consisting of —NR¹³R¹⁴,—N⁺(O⁻)R¹³R¹⁴ and —N(OH)R¹³; and R¹³ and R¹⁴ are independently selected from the group consisting of hydrogen, alkyl, lower alkyl substituted with hydroxy, alkylamino, cyanoalkyl, cycloalkyl, aryl and heteroaryl; or R¹³ and R¹⁴ may combine to form a heteroalicyclic or heteroaryl group.

74. The formulation of claim 1, wherein R⁶ is —C(O)R¹⁰ wherein R¹⁰ is —NR¹¹(CH₂)ₙR¹² wherein:
R₁₁ is hydrogen or lower unsubstituted alkyl;
n is 2 or 3; and
R¹² is —NR¹³R¹⁴ wherein R¹³ and R¹⁴ are independently unsubstituted lower alkyl; and
R⁷ is selected from the group consisting of hydrogen, alkyl, aryl and heteroaryl.

75. The formulation of claim 1, wherein R⁶ is N-(2-dimethylaminoethyl)aminocarbonyl, N-(2-diethylaminoethyl)-N-methylaminocarbonyl, N-(3-dimethylaminopropyl)aminocarbonyl, N-(2-diethylaminoethyl)aminocarbonyl, N-(2-ethylaminoethyl)aminocarbonyl, N-(3-ethylaminopropyl)aminocarbonyl, N-(2-hydroxy-3-pyrrolidin-1-yl-propyl)-aminocarbonyl, N-(2-pyrrolidin-1-yl-ethyl)-aminocarbonyl or N-(3-diethylaminopropyl)aminocarbonyl.

76. The formulation of claim 1, wherein the compound of formula is selected from the group consisting of:

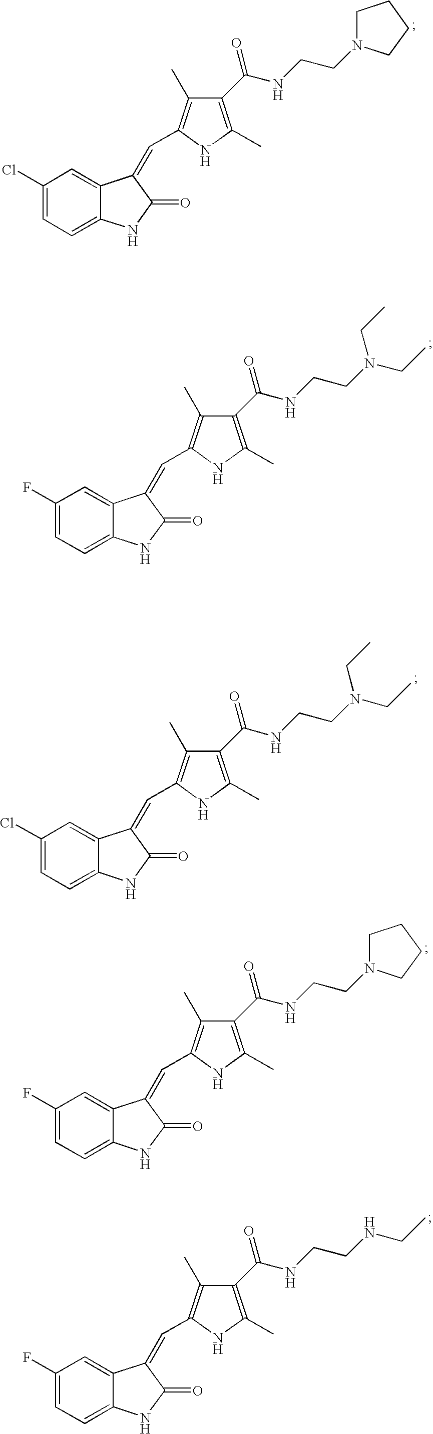

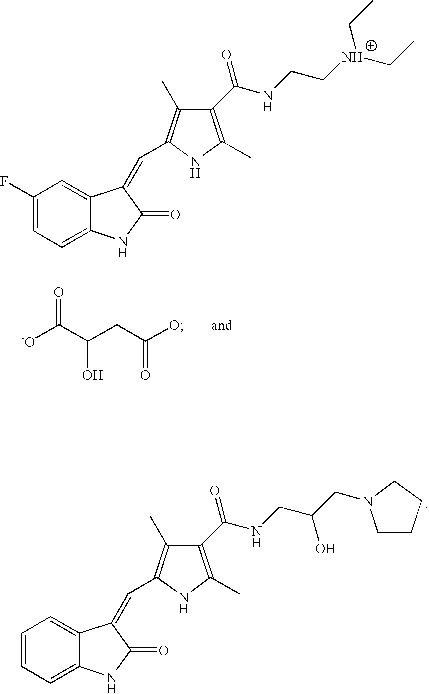

77. The formulation of claim 1, wherein the compound of formula I is:

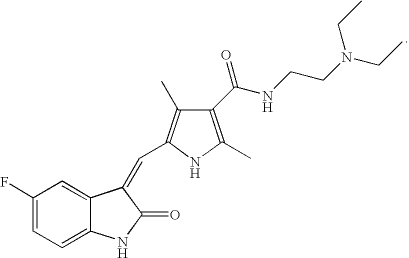

78. The formulation of claim 1, wherein said formulation comprises 15-60% w/w of an indolinone of formula I, or a pharmaceutically acceptable salt thereof, 25-75% w/w mannitol, 4-8% w/w croscaramellose sodium, 4-6% w/w povidone and 0.5-2% w/w magnesium stearate.

79. The formulation of claim 1, wherein said formulation comprises 30-50% w/w of an indolinone of formula I, or a pharmaceutically acceptable salt thereof, 35-60% w/w mannitol, 5-8% w/w croscaramellose sodium, 4-6% w/w povidone and 1-2% w/w magnesium stearate.

80. The formulation of claim 1, wherein said formulation comprises 40% w/w of an indolinone of formula I, or a pharmaceutically acceptable salt thereof, 47.5% w/w mannitol, 6% w/w croscararnellose sodium, 5% w/w povidone and 1.5% w/w magnesium stearate.

81. The formulation of claim 1, wherein said formulation comprises 10-16% w/w of an indolinone of formnula I, or a pharmaceutically acceptable salt thereof, 65-80% w/w mannitol, 5-10% w/w croscaramellose sodium, 4-8% w/w povidone and 1-2% w/w magnesium stearate.

82. The formulation of claim 1, wherein said formulation comprises 15.2% w/w of an indolinone of formula I, or a pharmaceutically acceptable salt thereof, 72.7% w/w mannitol, 6% w/w croscaramellose sodium, 5.1% w/w povidone and 1% w/w magnesium stearate.

83. The formulation of claim 1, wherein said formulation comprises 38-42% w/w of an indolinone of formula I, or a pharmaceutically acceptable salt thereof, 45-49% w/w mannitol, 4-8% w/w croscaramellose sodium, 3-7% w/w povidone and 1.3-1.7% w/w magnesium stearate.

84. The formulation of claim 1, wherein said formulation comprises 39-43% w/w of an indolinone of formula I, or a pharmaceutically acceptable salt thereof, 46-50% w/w mannitol, 5-9% w/w croscaramellose sodium, 4-8% w/w povidone and 1.4-1.8% w/w magnesium stearate.

85. The formulation of claim 1, wherein said formulation comprises 38-41% w/w of an indolinone of formula I, or a pharmaceutically acceptable salt thereof, 45-48% w/w mannitol, 4-7% w/w croscararnellose sodium, 3-6% w/w povidone and 1.3-1.6% w/w magnesium stearate.

86. The formulation of claim 1, wherein said formulation comprises 39-43% w/w of an indolinone of formula I, or a pharmaceutically acceptable salt thereof, 46-50% w/w mannitol, 5-9% w/w croscaramellose sodium, 4-8% w/w povidone and 1.4-1.8% w/w magnesium stearate.

87. The formulation of claim 1, wherein said formulation comprises 39-41% w/w of an indolinone of formula I, or a pharmaceutically acceptable salt thereof, 46-48% w/w mannitol, 5-7% w/w croscaramellose sodium, 4-6% w/w povidone and 1.4-1.6% w/w magnesium stearate.

88. The formulation of claim 1, wherein said formulation comprises 39-43% w/w of an indolinone of formula I, or a pharmaceutically acceptable salt thereof, 46-50% w/w mannitol, 5-9% w/w croscaramellose sodium, 4-8% w/w povidone and 0.8-1.5% w/w magnesium stearate.

89. The formulation of claim 1, wherein said formulation comprises 39-43% w/w of an indolinone of formula I, or a pharmaceutically acceptable salt thereof, 46-50% w/w mannitol, 5-9% w/w croscaramellose sodium, 4-8% w/w povidone and 0.8-1.2% w/w magnesium stearate.

90. A solid formulation, said formulation comprising 5-60% w/w of an indolinone of formula I: 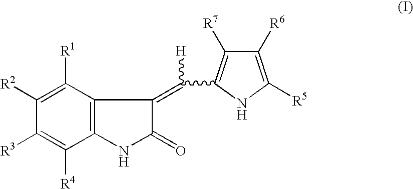
wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂)ₙR¹² or —NHCH₂CH(OH)CH₂R₁₂;
R¹¹ is selected from the group consisting of hydrogen and alkyl;
R¹² is selected from the group consisting of —NR¹³R¹⁴,—N⁺(O⁻)R¹³R¹⁴; —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4; or
pharmaceutically active salts of the-compound of formula I; and
a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants;
with the proviso that said formulation does not comprise a surfactant and/or a flow enhancer.

91. A solid formulation, said formulation consisting essentially of 5-60% w/w of an indolinone of formula I: 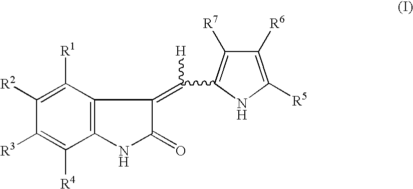
wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴,—NR¹³C(O)R¹⁴,—C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂)ₙR¹² or —NHCH₂CH(OH)CH₂R₁₂;
R¹¹ is selected from the group consisting of hydrogen and alkyl;
R¹² is selected from the group consisting of —NR¹³R¹⁴, —N⁺(O⁻)R¹³R¹⁴, —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4; or
pharmaceutically active salts of the compound of formula I; and
a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants.

92. A solid formulation, said formulation comprising 5-60% w/w of the malate salt of an indolinone of formula I: 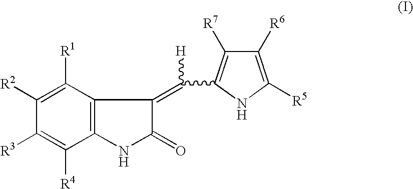
wherein:
R¹ is selected from the group consisting of hydrogen, halo, alkyl, cycloalkyl, aryl, heteroaryl, heteroalicyclic, hydroxy, alkoxy, —(CO)R¹⁵,—NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴,—C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴,—NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂)ₙR¹² or —NHCH₂CH(OH)CH₂R₁₂;
R¹¹ is selected from the group consisting of hydrogen and alkyl;
R¹² is selected from the group consisting of —NR¹³R¹⁴, —N⁺(O⁻)R¹³R¹⁴, —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4; and
a pharmaceutically acceptable carrier therefor comprising 10-86% w/w of one or more pharmaceutically acceptable diluents, 2-20% w/w of one or more pharmaceutically acceptable binders, 2-20% w/w of one or more pharmaceutically acceptable disintegrants, and 1-10% w/w of one or more pharmaceutically acceptable lubricants.

93. A solid formulation comprising an indolinone compound of formula I: 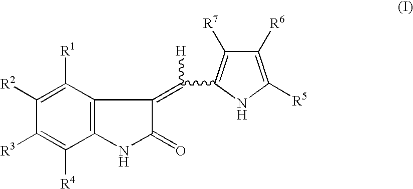
wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂)ₙR¹² or —NHCH₂CH(OH)CH₂R₁₂;
R¹¹ is selected from the group consisting of hydrogen and alkyl;
R¹² is selected from the group consisting of —NR¹³R¹⁴, —N⁺(O⁻)R¹³R¹⁴, —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4; or
pharmaceutically active salts of the compound of formula I;
wherein the bulk density of said formulation is at least about 0.50 kg/L.

94. The formulation of 93, wherein the bulk density of said solid formulation is about 2 to about 8 fold higher than the bulk density of the indolinone compound by itself.

95. The formulation of 93, wherein the bulk density of said formulation is about 0.55, 0.56, 0.57, 0.58, 0.59. 0.60, 0.61, 0.62, 0.63, 0.64, 0.65, 0.66, 0.67, 0.69 or 0.7 kg/L.

96. The formulation of one of claims 93, wherein the ratio of tap density to bulk density of said formulation is from about 1.10 to about 1.30.

97. The formulation of claim 93, wherein the ratio is from about 1.10 to about 1.33, or about 1.10 to about 1.25, or about 1.10 to about 1.20, or about 1.10 to about 1.15.

98. The formulation of claim 93, wherein said salt of said compound is 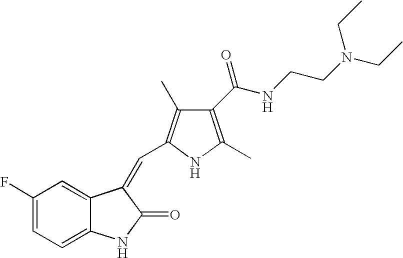
or the L-malate salt thereof.

99. The formulation of claim 93, wherein said formulation comprises 15-40% of the indolinone compound.

100. The formulation of claim 93, wherein said formulation comprises 40% w/w of an indolinone of formula I, or a pharmaceutically acceptable salt thereof, 47.5% w/w mannitol, 6% w/w croscaramnellose sodiumn, 5% w/w povidone and 1.5% w/w magnesium stearate.

101. A solid formulation comprising an indolinone compound of formula I: 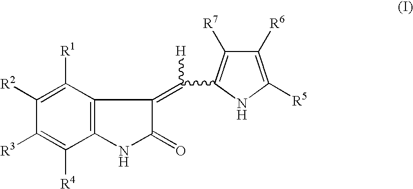
wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R^{14,} —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂)ₙR¹² or —NHCH₂CH(OH)CH₂R₁₂;
R¹¹ is selected from the group consisting of hydrogen and alkyl;
R¹² is selected from the group consisting of —NR¹³R¹⁴, —N⁺(O⁻)R¹³R¹⁴, —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4; or
pharmaceutically active salts of the compound of formula I;
wherein said formulation is in particulate form, and wherein no more than 55% of the particles have a size less than 250 microns.

102. The formulation of claim 101, wherein said salt of said compound is 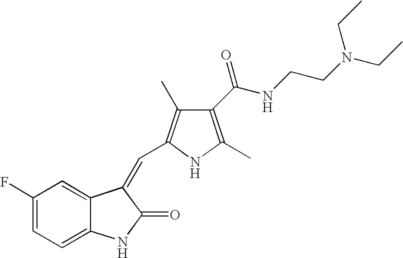
or the L-malate salt thereof.

103. The formulation of claim 101, wherein said formulation comprises 15-40% of the indolinone compound.

104. The formulation of claim 101, wherein said formulation comprises 40% w/w of an indolinone of formula I, or a pharmaceutically acceptable salt thereof, 47.5% w/w mannitol, 6% w/w croscaramellose sodium, 5% w/w povidone and 1.5% w/w magnesium stearate.

105. A solid formulation comprising an indolinone compound of formula I: 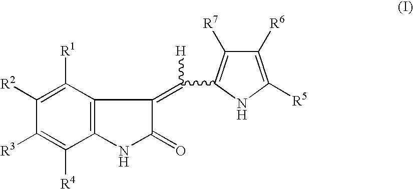
wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂),R¹² or —NHCH₂CH(OH)CH₂R₁₂;
R¹¹ is selected from the group consisting of hydrogen and alkyl;
R¹² is selected from the group consisting of —NR¹³R¹⁴, —N⁺(O⁻)R¹³R¹⁴, —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4; or
pharmaceutically active salts of the compound of formula I;
wherein said formulation is in particulate form, and wherein the mean particle size is between 106 and 250 microns.

106. A solid formulation comprising the malate salt of an indolinone compound of formula I: 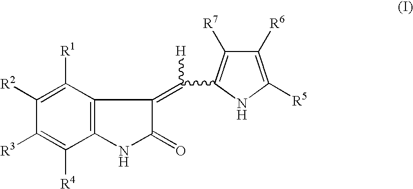
wherein:
R¹ is selected from the group consisting of hydrogen, halo, alkyl, cycloalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R¹⁰; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is —N(R¹¹)(CH₂)ₙR¹² or —NHCH₂CH(OH)CH₂R₁₂;
R¹¹ is selected from the group consisting of hydrogen and alkyl;
R¹² is selected from the group consisting of —NR¹³R¹⁴, —N⁺(O⁻)R¹³R¹⁴, —N(OH)R¹³, and —NHC(O)R¹³;
R¹³ and R¹⁴ are independently selected from the group consisting of hydrogen, alkyl, cyanoalkyl, cycloalkyl, aryl and heteroaryl; or
R¹³ and R¹⁴ may combine to form a heteroalicyclic or heteroaryl group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4;
wherein the bulk density of said solid formulation is about 2 to about 8 fold higher than the bulk density of the malate salt of the indolinone compound by itself.
